(19) United States
(12) Patent Application Publication
(10) Pub. No.: US 2012/0299474 A1
(43) Pub. Date: Nov. 29, 2012

(54) LIGHT EMITTING TETRAPHENYLENE DERIVATIVES, ITS METHOD FOR PREPARATION AND LIGHT EMITTING DEVICE USING THE SAME DERIVATIVES

(75) Inventors: Benzhong Tang, Hong Kong (CN); Zujin Zhao, Hong Kong (CN); Ka Wai Jim, Hong Kong (CN); Wing Yip Lam, Hong Kong (CN); Shuming Chen, Hong Kong (CN); Hoi Sing Kwok, Hong Kong (CN)

(73) Assignee: THE HONG KONG UNIVERSITY OF SCIENCE AND TECHNOLOGY, Hong Kong (CN)

(21) Appl. No.: 13/577,155

(22) PCT Filed: Mar. 1, 2011
(86) PCT No.: PCT/CN2011/000329

§ 371 Date: Aug. 3, 2012

Related U.S. Application Data
(60) Provisional application No. 61/282,555, filed on Mar. 1, 2010.

(51) Int. Cl. H05B 33/14; C07D 403/10; C07D 417/14; C07D 417/10; C07D 285/14; H05B 33/10; C07D 217/02; C07C 15/18; C07C 15/30; C07C 15/24; C07C 15/38; C07C 15/28; C07C 211/54; C07D 209/86

(52) U.S. Cl. 313/504; 564/315; 548/440; 548/134; 548/446; 546/144; 585/26; 585/24; 427/66

(57) ABSTRACT

Provided are a light emitting material comprising one or more tetraphenylethene (TPE) derivatives of formula (1a) with high thermal stability and high solid quantum yield efficiency, and an electroluminescent or light emitting device such as OLED comprising the same TPE derivatives and a method of preparing the same.

### FIELD OF THE INVENTION

The present subject matter relates to a light-emitting material and the use of said material in a light-emitting device capable of converting electric energy to light. In particular, the presently described subject matter relates to a light emitting material comprising tetraphenylethene derivatives and the use of the same in light emitting devices, such as organic light-emitting diodes (OLEDs).

### BACKGROUND OF THE INVENTION

Synthesis of luminescent materials with efficient light emissions has been of interest to many scientists for many years. While the advancements in electronics and optics, such as organic light-emitting diodes (OLEDs), are directly associated with is the development of new luminescent materials, there has been a thirst for this kind of material in the optoelectronic industry. (Chem. Rev. 2007, 107, 1011, Nature 1998, 395, 151).

About half a century ago, Förster and Kasper discovered that the fluorescence of pyrene weakens with an increase in solution concentration. It was soon recognized that this was a general phenomenon for many aromatic compounds. This concentration-quenching effect was found to be caused by the formation of sandwich-shaped excimers and exciplexes aided by the collisional interactions between the aromatic molecules in the excited and ground state which are known to be common to most aromatic compounds and their derivatives. This phenomenon is also observed when the molecule is in its solid state, because there is no “solvent” in the solid state and the “solute” molecules are located in the immediate vicinity. The aromatic rings of the neighboring fluorophores, especially those with disc-like shapes, experience strong π-π stacking interactions, which promotes the formation of aggregates with ordered or random structures. The excited states of the aggregates often decay via non-radiative pathway, known as aggregation-caused quenching (ACQ) of light emission in the condensed phase.

Whereas luminescence behaviors of molecules are normally investigated in the solution state, they are practically used as materials in the solid state. The ACQ effect, however, comes into play in the solid state, which has prevented many lead luminogens identified by the laboratory solution-screening process from finding real-world applications in an engineering robust form.

To mitigate the ACQ effect, various chemical (Chem. Commun. 2008, 1501. Chem. Commun. 2008, 217.), physical and engineering (Langmuir 2006, 22, 4799. Macromolecules 2003, 36, 5285.) approaches and processes have been developed. The attempts, however, have met with only limited success. The difficulty is in the fact that aggregate formation is an intrinsic process when luminogenic molecules are located in close vicinity in the condensed phase. Accordingly, needed in the art was a system in which light emission is enhanced, rather than quenched, by aggregation.

In 2001, the present inventors developed such a system, in which luminogen aggregation played a constructive, instead of a destructive, role in the light emitting process. The inventors also observed a novel phenomenon and coined the term “aggregation-induced emission” (AIE) since the non-luminescent molecules were induced to emit by aggregate formation: a series of propeller-like, non-emissive molecules, such as silole and tetraphenylethene (TPE), were induced to emit intensely by aggregate formation (Chem. Commun. 2001, 1740, J. Mater. Chem. 2001, 11, 2974, Chem. Commun. 2009, 4332, Appl. Phys. Lett. 2007, 91, 011111.). After this discovery, through extensive exploration in this area, the present inventors discovered a large number of molecules bearing this novel property. In addition, through a series of designed experiments, and theoretical calculations, the present inventors identified restriction of intramolecular rotation (IMR) as the main cause for the AIE effect (J. Phys. Chem. B 2005, 109, 10061, J. Am. Chem. Soc. 2005, 127, 6335).

Among the prepared AIE molecules, TPE enjoys the advantages of facile synthesis and efficient photoluminescence as well as high thermal stability. A wide variety of substituents have been attached into its phenyl blades to endow it with enhanced and/or new electronic and optical properties. As a result, a method that can help to solve the quenching problem faced by many dyes which are strongly emissive in solution but become quenched in their solid states, have been developed and presently described in this application.

### SUMMARY OF THE INVENTION

The present subject matter, in one aspect provides a light-emitting material comprising one or more tetraphenylethene (TPE) derivatives having the formula (1a) with high thermal stability. With the material, the solid state quantum yield efficiency can reach as high as unity.

In another aspect, the present subject matter provides an electroluminescent (EL) device or a light emitting device (LED), comprising highly emissive TPE derivatives. The energy source of an EL device or LED is electricity. In one embodiment, an OLED comprising an anode, a cathode and one or more organic layer(s) located between them is provided wherein the organic layer comprises a light emitting material comprising one or more TPE derivatives in the structure.

In another aspect, the present subject matter provides a method of preparing a light emitting device comprising an anode, a cathode and one or more organic layers located between the anode and the cathode, which comprises thermally evaporating the organic layer in sequence in a multi-source vacuum chamber at a base pressure, wherein the organic layer comprises a light emitting material comprising one or more TPE derivatives.

The TPE derivatives are non-emissive or weakly fluorescent in their solution state. However, the fluorescent intensity is greatly enhanced when the molecules act as nanoparticle suspensions in poor solvents or are fabricated into a thin film. The propeller-shaped TPE core can help to prevent strong packing between molecules and can help to solve the aggregation-caused quenching problem encountered by many dye molecules. This concept can be used to obtain a wide variety of highly emissive molecules for the use of optoelectronic devices such as OLEDs. The provided concept can be further applied for the preparation of various kinds of emitting molecules by changing the pendants of the molecules.

The preparation of the materials is simple and all the materials can be obtained in high yields. Due to the large amount of aromatic rings in the structure, all the dye molecules show high thermal stability. The molecules show strong fluorescence in their solid states. The electroluminescence of the molecules shows excellent results, and thus the molecules can be used for organic light-emitting diodes.

### BRIEF DESCRIPTION OF THE FIGURES

FIG. 1 (a) shows absorption spectra of 1-6 in THF solutions. FIG. 1 (b) shows photoluminescence (PL) spectra of 1 in THF/water mixtures with different water contents. Photographs of 1 in THF/water mixtures with 0 (left) and 90% (right) water contents taken under UV illumination are shown. The spectrum shows excitation wavelength of 350 nm.

FIG. 2 (a) shows molecular orbital amplitude plots (MOAP) of highest occupied molecular orbital (HOMO) and lowest occupied molecular orbital (LUMO) levels of 4, 3, 1, and 2, calculated using the B3LYP/6-32G* basis set. FIG. 2 (b) shows MOAP of HOMO and LUMO energy levels of 5 and 6 calculated using B3LYP/6-31G* basis set.

FIG. 3 shows C—H . . . π hydrogen bonds with indicated distances (Å) between TPE-Ar adjacent molecules (upper panel) and shows top view of adjacent TPE-Ar molecules (lower panel).

FIG. 4 (a) shows plots of luminance and current density vs. voltage in multilayer light-emitting diodes of 1 and 2 with a device configuration of ITO/NPB/1 or 2/TPBi/Alq₃/LiF/Al. FIG. 4 (b) shows plots of external quantum efficiency vs. current density in multilayer light-emitting diodes of 1 and 2 with a device configuration of ITO/NPB/1 or 2/TPBVAlq₃/LiF/Al.

FIG. 5 shows Oakridge Thermal Ellipsoid Plot (“ORTEP”) drawings of TPE-Ars.

FIG. 6 (a) shows PL spectra of 1 and 2 in THF solutions (10 M). FIG. 6 (b) shows PL spectra of crystals of TPE-Ars and FIG. 6 (c) shows amorphous films of TPE-Ars.

FIG. 7 (a) shows electroluminescence (EL) spectra of 1-6 in multilayer light-emitting diodes of TPE-Ars with a device configuration of ITO/NPB/TPE-Ar/TPBi/Alq₃/LiF/Al. FIG. 7 (b) to FIG. 7 (d) show current efficiency vs. current density, luminance vs. voltage, and current density vs. voltage of 1-6, respectively, in multilayer light-emitting diodes of TPE-Ars with a device configuration of ITO/NPB/TPE-Ar/TPBi/Alq₃/LiF/Al.

FIG. 8 shows molecular structure of 7 and its molecular orbital amplitude plots of HOMO and LUMO energy levels calculated by semiempirical Parameterized Model number 3 (PM3) method.

FIG. 9 (a) shows absorption spectrum of 7 in THF solution. FIG. 9 (b) shows PL spectra of 7 in THF/water mixtures (10⁻⁶ M). FIG. 9 (c) shows Thermogravimetric analysis (TGA) and Differential Scanning calorimetry (DSC) thermograms of 7 recorded under nitrogen at a heating rate of 10° C./min. FIG. 7 (d) shows PL spectra of amorphous film and crystalline powders of 7 and EL spectra of 7 in devices A and B. Excitation wavelength is shown at 350 nm.

FIG. 10 (a) and FIG. 10 (b) respectively show fluorescence decay curves of THF solution (10⁻⁶M) and crystalline powders of 7 at different temperatures.

FIG. 11 (a) shows changes in luminance and current density with applied biases in multilayer EL devices of 7. FIG. 11 (b) shows external quantum and current efficiencies vs. current density in multilayer EL devices of 7.

FIG. 12 shows Matrix Assisted Laser Desorption/lonization Time-of-Flight (MALDI-TOF) mass spectrum of 7.

FIG. 13 shows X-ray Powder Diffraction (XRD) diffractogram of as-prepared powders of 7.

FIG. 14 (a) and FIG. 14 (b) respectively show absorption spectra and PL spectra of 7 in THF solutions with a concentration of 10⁻⁵, 10⁻⁶ and 10⁻⁷ M.

FIG. 15 shows PL spectra of 7 in THF solution (10⁻⁶ M) at 298 and 77 K.

FIG. 16 (a) shows PL spectra of the powders of 7 at 298 and 77 K. FIG. 16(b) shows PL spectra of the film of 7 at 298 and 77 K.

FIG. 17 shows ORTEP drawings and B3LYP/6-31G* calculated molecular orbital amplitude plots of HOMO and LUMO levels of 8 and cis-9.

FIG. 18 (a) and FIG. 18 (b) respectively show normalized PL spectra of 8 and 9 in THF solutions with different concentrations. FIG. 18 (c) and FIG. 18 (d) respectively show PL spectra of 8 and 9 in THF/water mixtures (1 μM) with different water contents. Inserted in the panels of FIG. 18 (c) and FIG. 18 (d) are photographs of 8 and 9 in THF/water mixture with 0 (left) and 99.5% (right) water contents taken under UV illumination. Excitation wavelength is 350 nm.

FIG. 19 (a) and FIG. 19 (d) show hydrogen bonds and π-π interactions with indicated distances (A) between adjacent molecules of 8 and cis-9. FIG. 19 (b) and FIG. 19 (e) show side views of, and FIG. 19 (c) and FIG. 19 (f) show top views of adjacent molecules of 8 and cis-9 along the plane of pyrene stacking, respectively.

FIGS. 20 (a) and (c) show plots of luminance and current density vs. voltage and FIGS. 20 (b) and (d) show current efficiency vs. current density curves, in multilayer devices with configurations of ITO/NPB/8 or 9/TPBi/LiF/Al and ITO/NPB/9 or Alq₃/TPBi/Alq₃/LiF/Al. Inset in panel d: electroluminescence spectra.

FIG. 21 shows absorption spectra of 8 and 9 in THF solutions (10 μM).

FIG. 22 (a) and FIG. 20 (b), respectively, show concentration-dependent PL spectra of 8 and 9 in THF solutions. Excitation wavelength is shown at 350 nm.

FIG. 23 (a) and FIG. 23 (b), respectively, show PL spectra of amorphous films of 8 and 9 and EL spectra of 8 and 9, in multilayer devices with a configuration of ITO/NPB(60 nm)/8 or 9(20 nm)/TPBi(30 nm)/LiF(1 nm)/Al(100 nm).

FIG. 24 shows electron diffraction (ED) patterns of crystalline aggregates of 8 (left) and 9 (right) formed in THF/water mixtures containing 90% water.

FIG. 25 shows non-efficient overlapping between pyrene rings in cis-9 crystals.

FIG. 26 shows plots of external quantum efficiency versus current density in multilayer devices with a configuration of ITO/NPB(60 nm)/9 or Alq₃(20 nm)/TPBi(10 nm)/Alq₃(30 nm)/LiF(1 nm)/Al(100 nm).

FIG. 27 (a) shows emission spectra of THF solution of 10 (10 μM) and its aggregates suspended in THF/water mixtures with different fractions of water (f_{w} 70-99.5 vol %), and FIG. 27 (b) shows emission spectra of the amorphous film and crystalline fibre of 10 in the solid state.

FIG. 28(a) and FIG. 28 (b) show SEM images of the microfibers of 10 obtained by slow evaporation of its THF/ethanol solutions on cupper grids. FIG. 28 (c) shows optical image of the microfibers of 10 obtained by slow evaporation of its THF/ethanol solutions on quartz plates. FIG. 28 (d) to FIG. 28 (f) show fluorescent images of the microfibers of 10 obtained by slow evaporation of its THF/ethanol solutions on quartz plates.

FIG. 29 (a) and FIG. 29 (b), respectively, show plots of luminance vs. voltage and current efficiency vs. current density, in the 10-based multilayer light-emitting diodes with device configuration of ITO/NPB/10/TPIBi/Alq₃/LiF/Al. Inset in panel B: electroluminescence spectra. The (10, Alq₃) layers in devices I and II are (20 nm, 30 nm) and (40 nm, 10 nm) in thickness, respectively.

FIG. 30 (a) shows ED patterns of amorphous aggregates of 10 formed in THF/water mixtures with water contents of 80 vol %. FIG. 30 (b) shows ED patterns of crystalline aggregates of 10 formed in THF/water mixtures with water contents of 70 vol %. FIG. 30 (c) shows high resolution TEM image of the surface of aggregates of 10 formed in a THF/water mixture with 70% water fraction.

FIG. 31 shows XRD patterns of crystalline fibers of 10.

FIG. 32 (a) shows plots of current density vs. voltage and FIG. 32 (b) shows external quantum efficiency vs. current density, for 10-based multilayer electroluminescence devices with a configuration of ITO/NPB/10/TPBi/Alq3/LiF/Al.

FIG. 33 shows a schematic illustration of the 10 based device structures, as well as the energy level and molecular structure of 10.

FIG. 34 shows PL spectrum of BTPE (10) as well as absorption spectrum of DCJTB and C545T.

FIG. 35 (a) shows current density-luminance-voltage of the fabricated devices using 10.

FIG. 35 (b) shows current efficiency-current density characteristics of the fabricated devices using 10. FIG. 35 (c) shows EL spectra of the fabricated devices using 10.

FIG. 36 (a) and FIG. 36 (b) respectively show EL spectra of the WOLEDs, without and with 2 nm thick NPB electron-blocking layer.

FIG. 37 shows a schematic illustration of the 7 and 12 based device structures as well as the energy level and molecular structures thereof.

FIG. 38 (a) and FIG. 38 (b) respectively show voltage-luminance-current density characteristics of the 7 and 12 based devices and EL efficiency-current density characteristics of the 7 and 12 based devices.

FIG. 39 (a) shows 7 and 12 based EL spectra of the bluish-green, red and white 1 devices. FIG. 39 (b) shows EL spectra of white 2 devices under different driving voltages and FIG. 39 (c) shows photos of bluish-green, red and white 2 devices.

FIG. 40 (a) and FIG. 40 (b) respectively show photos of p-16 and o-16 in THF solutions (1 μm) under illumination of a UV lamp.

FIG. 41 shows ORTEP drawings of o-16.

FIG. 42 shows molecular structure of o-16 and its molecular orbital amplitude plots of HOMO and LUMO energy levels calculated by semiempirical PM3 method.

FIG. 43 (a) and FIG. 43 (b) respectively show photos of p-17 and o-17 in THF solutions (1 μm) under illumination of a UV lamp.

### DETAILED DESCRIPTION OF THE INVENTION

### Definitions

The following definitions are provided for the purpose of understanding the present subject matter and for constructing the appended patent claims.

It is noted that, as used in this specification and the appended claims, the singular forms “a,” “an” and “the” include plural references unless the context clearly dictates otherwise.

“Alkyl” refers to, unless otherwise specified, an aliphatic hydrocarbon group which may be a straight or branched chain having about 1 to about 15 carbon atoms in the chain, optionally substituted by one or more atoms. A particularly suitable alkyl group has from 2 to 6 carbon atoms.

The term “unsaturated” refers to the presence of one or more double or triple bonds between atoms of a radical group.

“Heteroatom” refers to an atom selected from the group consisting of nitrogen, oxygen, sulfur, phosphorus, boron and silicon.

“Heteroaryl” as a group or part of a group refers to an optionally substituted aromatic monocyclic or multicyclic organic moiety of about 5 to about 10 ring members in which at least one ring member is a heteroatom.

“Cycloalkyl” refers to an optionally substituted non-aromatic monocyclic or multicyclic ring system of about 3 to about 10 carbon atoms.

“Heterocycloalkyl” refers to a cycloalkyl group of about 3 to 7 ring members in which at least one ring member is a heteroatom.

“Aryl” as a group or part of a group refers to an optionally substituted monocyclic or multicyclic aromatic carbocyclic moiety, preferably of about 6 to about 18 carbon atoms, such as phenyl, naphthyl, anthracene, tetracence, pyrene, etc.

“Heteroalkyl” refer to an alkyl in which at least one carbon atom is replaced by a heteroatom.

“Vinyl” refers to the presence of a pendant vinyl group (CH₂═CH—) in the structure of the molecules or the material described herein.

“Acetyl” refers to the presence of a pendant acetyl group (COCH₃) in the structure of the molecules or the material described herein.

Unless defined otherwise all technical and scientific terms used herein have the same meaning as commonly understood to one of ordinary skill in the art to which the presently described subject matter pertains.

Where a range of values is provided, for example, concentration ranges, percentage ranges, or ratio ranges, it is understood that each intervening value, to the tenth of the unit of the lower limit, unless the context clearly dictates otherwise, between the upper and lower limit of that range and any other stated or intervening value in that stated range, is encompassed within the described subject matter. The upper and lower limits of these smaller ranges may independently be included in the smaller ranges, and such embodiments are also encompassed within the described subject matter, subject to any specifically excluded limit in the stated range. Where the stated range includes one or both of the limits, ranges excluding either or both of those included limits are also included in the described subject matter.

Throughout the application, descriptions of various embodiments use “comprising” language; however, it will be understood by one of skill in the art, that in some specific instances, an embodiment can alternatively be described using the language “consisting essentially of” or “consisting of:”

For purposes of better understanding the present teachings and in no way limiting the scope of the teachings, unless otherwise indicated, all numbers expressing quantities, percentages or proportions, and other numerical values used in the specification and claims, are to be understood as being modified in all instances by the term “about.” Accordingly, unless indicated to the contrary, the numerical parameters set forth in the following specification and attached claims are approximations that may vary depending upon the desired properties sought to be obtained. At the very least, each numerical parameter should at least be construed in light of the number of reported significant digits and by applying ordinary rounding techniques.

### Abbreviation

NPB: 4,4′-bis[N-(1-napthyl-1-)-N-phenyl-amino]-biphenyl
ITO: Indium tin oxide
TPBi: 2,2′,2″-(1,3,5-benzinetriyl)tris(1-phenyl-1-H-benzimidazole)
Alq3: tris(8-hydroxyquinoline) aluminium
TPPyE: 1-pyrene-1,2,2-triphenylethene
TTPEPy: 1,3,6,8-tetrakis[4-(1,2,2-triphenylvinyl)phenyl]pyrene
BTPE: 4,4′-bis(1,2,2-triphenylvinyl)biphenyl
BTPETTD: 4-(4-(1,2,2-triphenylvinyl)phenyl)-7-(5-(4-(1,2,2-triphenyl)vinyl) thiophen-2-yl)benzo[c][1,2,5]thiadiazole
DCJTB: 4-(dicyanomethylene)-2-t-butyl-6(1,1,7,7-tetramethyljulolidyl-9-enyl)-4H-pyran
C545T: 10-(2-benzothiazolyl)-2,3,6,7-tetrahydro-1,1,7,7-tetramethyl-1H,5H,t1H-(1)-benzopyropyrano(6,7-8-i,j)quiholizin-11-one
BOLED: Blue organic light emitting diode
ROLED: Red organic light emitting diode
GOLED: Green organic light emitting diode
WOLED: White organic light emitting diode

Light Emitting Materials

The present subject matter relates to one or more light emitting materials comprising one or more moieties of formula (1a):

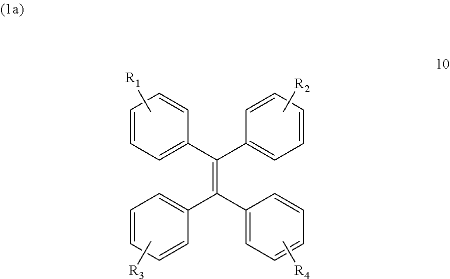

wherein R₁, R₂, R₃, and R₄, each independently of one another at each occurrence, are hydrogen or any organic or organometallic groups, with the proviso that at least one of R₁ to R₄ is not hydrogen; and when R₁ and R₄, or R₂ and R₃, are hydrogen, the other two of R₂ and R₃, or R₁ and R₄, are not phenyl groups.

In one embodiment, the moieties of formula (1a) described herein can be formed as single compounds or can be polymerized into compounds containing two or more moieties of formula (1a) joined together through one or more of the phenyl groups and one of the substituents R₁, R₂, R₃, and R₄.

In a further embodiment, each of R₁, R₂, R₃, and R₄ can independently form a fused cyclic moiety with the phenyl ring to which it is attached.

In another embodiment, each of R₁, R₂, R₃, and R₄ are independently at each occurrence hydrogen, alkyl, vinyl, acetyl, aryl, heteroaryl, cycloalkyl, heterocycloalkyl, or heteroaryl.

In a further embodiment, each of R₁, R₂, R₃, and R₄ are independently at each occurrence hydrogen, an optionally substituted C₂-C₆ alkyl, an optionally substituted vinyl group, an optionally substituted acetyl group, an optionally substituted aryl group having one or more rings of about 6 to about 14 carbon atoms, an optionally substituted heteroaryl group having one or more rings with 5 to 10 atoms in each ring and at least one heteratom in at least one ring, an optionally substituted cycloalkyl group having one or more rings with 3 to 10 carbon atoms in each ring, an optionally substituted heterocycloalkyl group having one or more rings with 3 to 7 atoms in each ring and at least one heteroatom in at least one ring, or an optionally substituted heteroaryl group having one or more rings with 5 to 10 atoms in each ring and at least one heteroatom in at least one ring.

In one embodiment in this regard, each of R₁, R₂, R₃, and R₄ can be an optionally substituted monocyclic or multicyclic organic moiety having 1, 2, 3, or 4 ring structures therein, for example, without limitation, phenyl, naphthyl, anthracene, tetracene, pyrene, carbazole, acridine, dibenzoazepine, quinoline, isoquinoline, and thiophene.

In still another embodiment, each of R₁, R₂, R₃, and R₄, independently of one another at each occurrence, can be selected from the group consisting of:

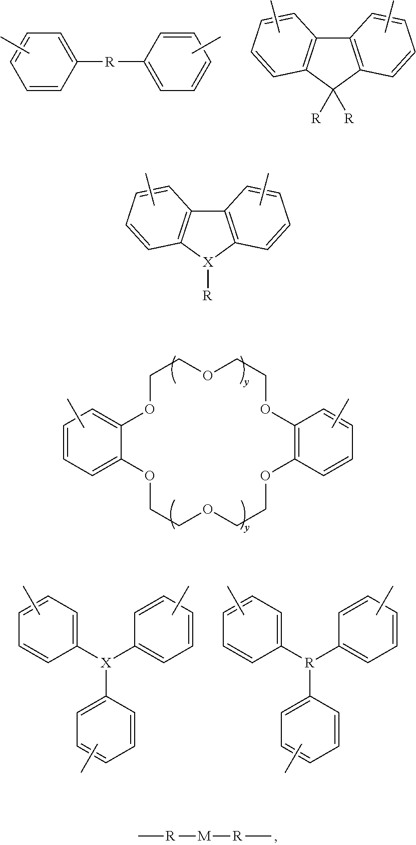

and hydrogen, wherein X is a heteroatom; y is an integer and is ≧1; R is alkyl, vinyl, acetyl, aryl, heteroaryl, cycloalkyl, heterocycloalkyl, or heteroalkyl that is optionally substituted; and M is a metal or organometallic compound.

In yet another embodiment, the light emitting materials described herein can be to selected from the group consisting of:

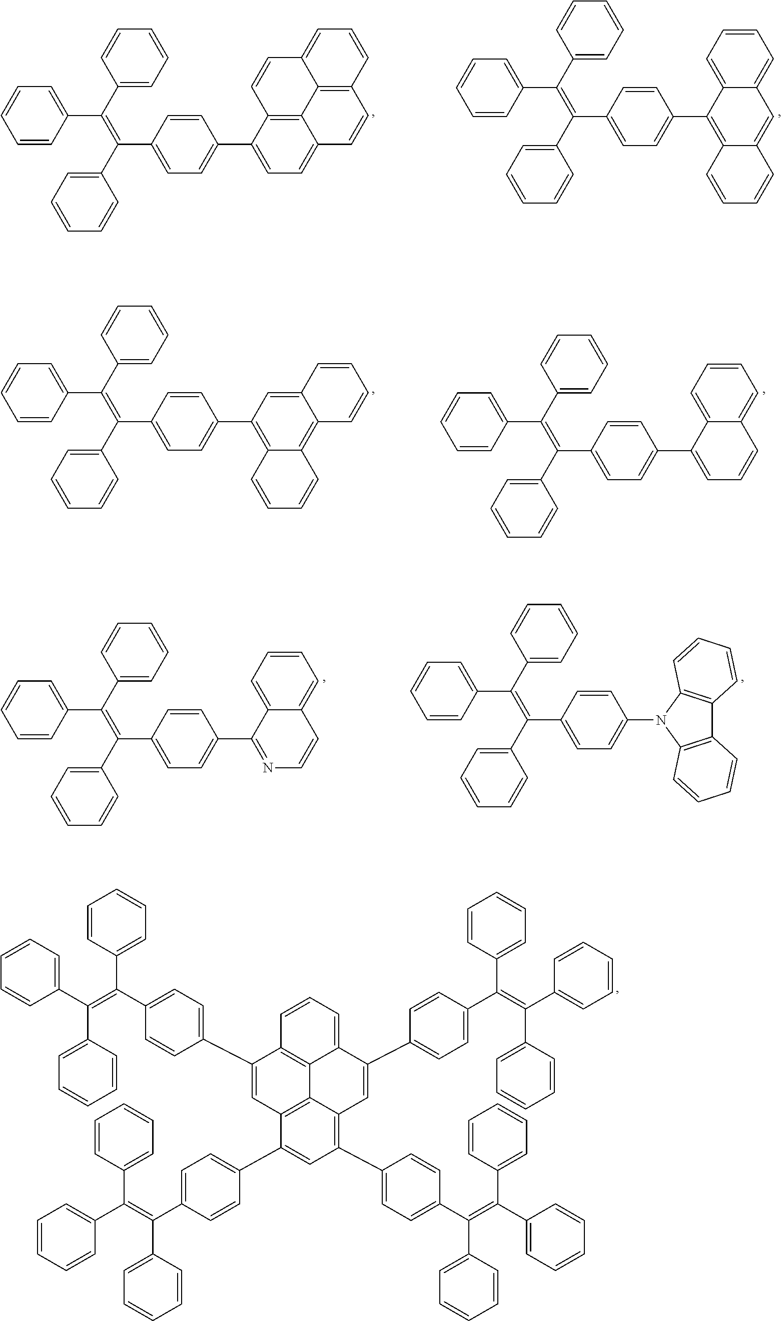

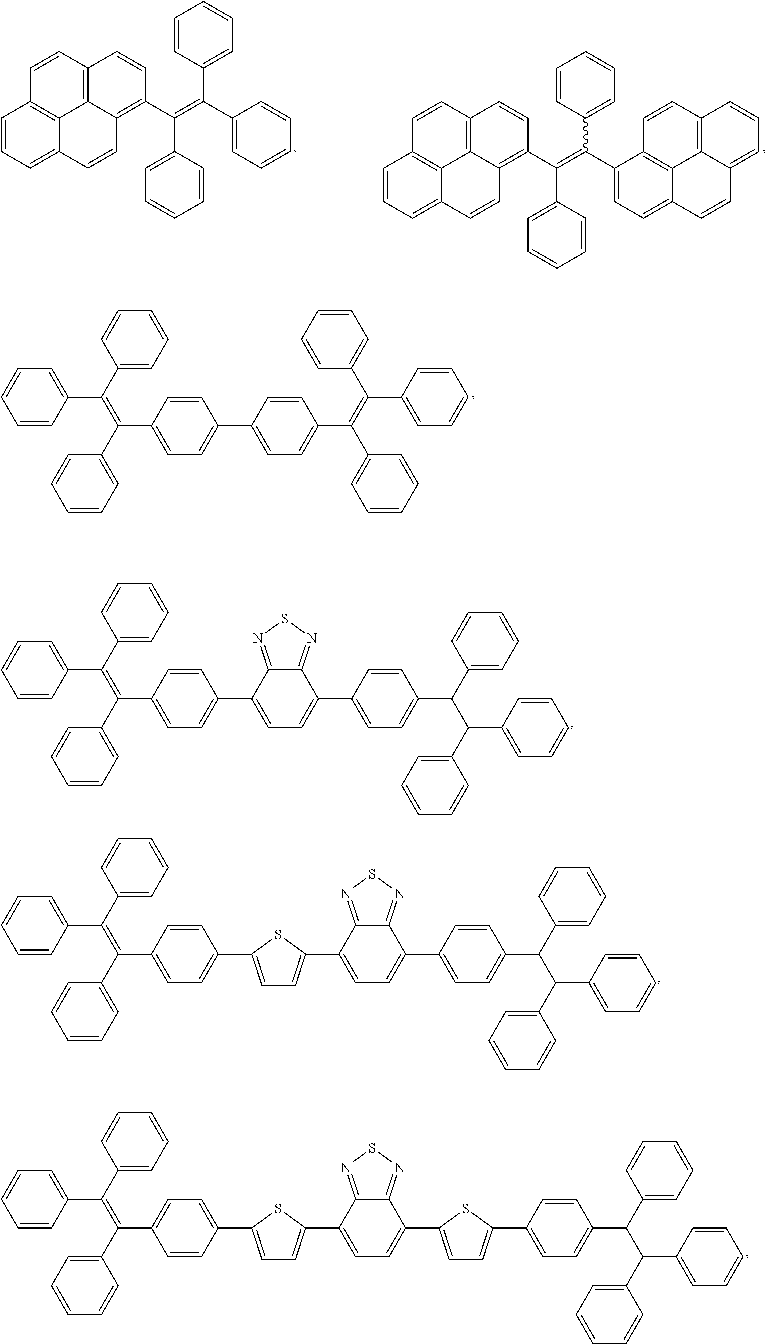

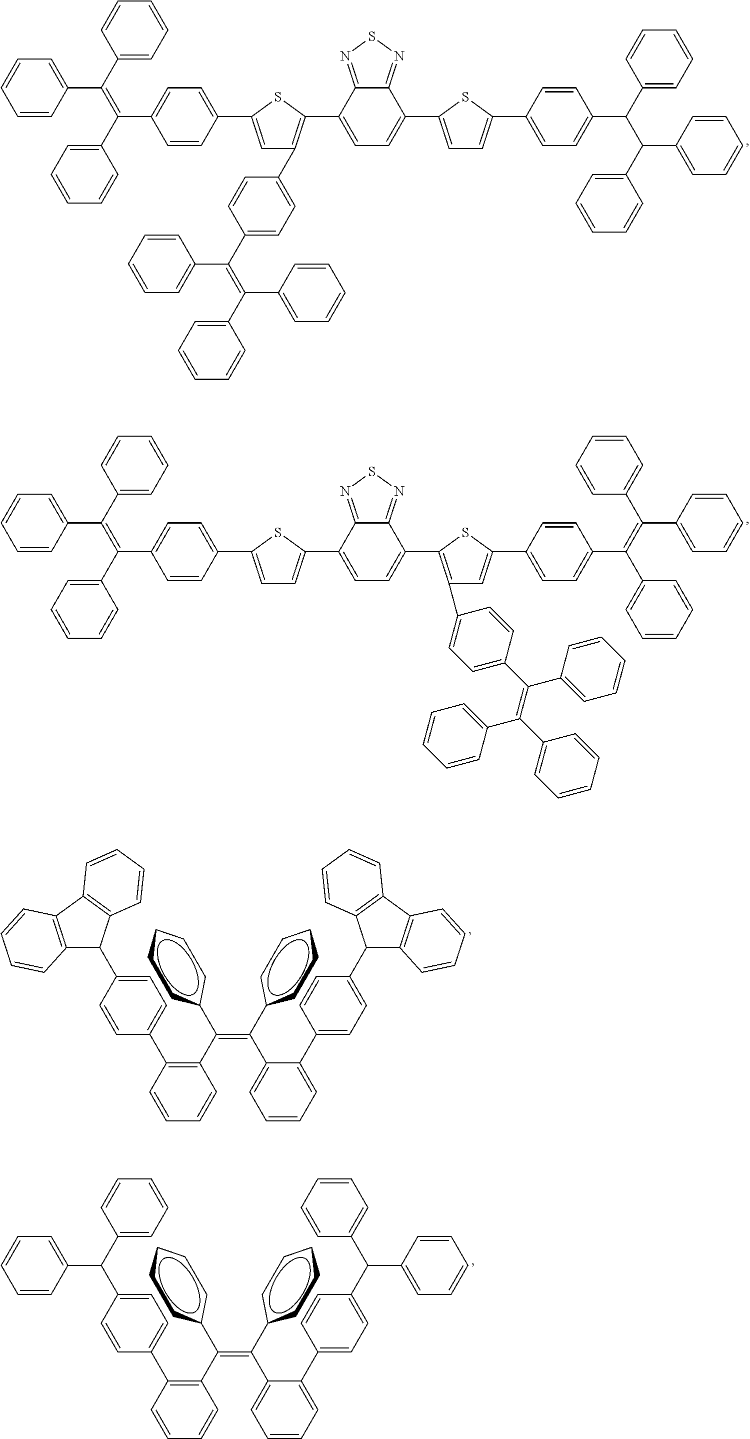

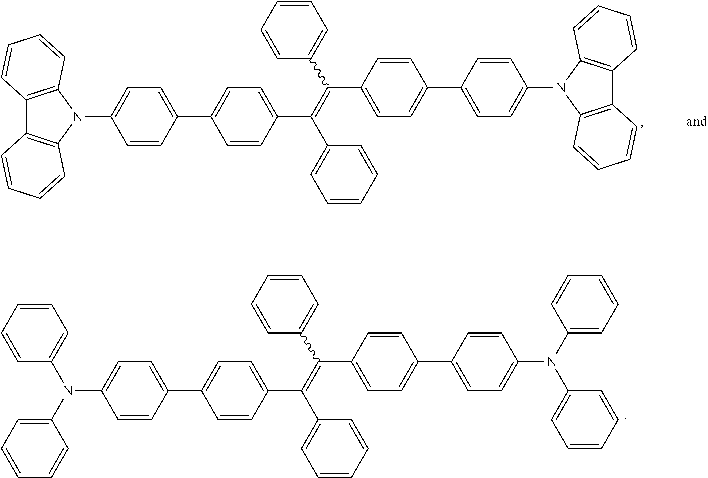

The TPE derivatives described herein are non-emissive or weakly fluorescent in their solution state, however the fluorescent intensity is greatly enhanced when the molecules act as nanoparticle suspensions in poor solvents or are fabricated into thin film. The propeller-shaped TPE core can help to prevent strong packing between molecules and can help to solve the aggregation-caused quenching problem encountered by many dye molecules. This concept can be used to obtain a wide variety of highly emissive molecules for the use of optoelectronic devices such as OLEDs. The provided concept can be further applied for the preparation of various kinds of emitting molecules by changing the pendants of the molecules.

In one embodiment, the light emitting materials described herein can have a molecular weight of at least about 300. In another embodiment, the light emitting materials described herein can have a molecular weight of between about 300 and about 3000. The light emitting materials described herein can further be in solid or crystalline form.

In another aspect, the herein described materials or molecules can be used to prepare an emitting layer of an organic light emitting device, an electroluminescent device, or another light emitting device.

The preparation of the materials or molecules is simple and all the materials can be obtained in high yields as shown below. Due to the large amount of aromatic rings in the structure, all the dye molecules show high thermal stability. The molecules show strong fluorescence in their solid states. The electroluminescence of the molecules shows excellent results, and thus the molecules can be used for organic light-emitting diodes.

In one aspect of the present subject matter, a light emitting material, such as dye molecules, comprising one or more tetraphenylethene derivatives having the structural formula of compound 29, in Scheme 1 below, and its preparation is provided, where R₁, R₂, R₃ and R₄, each independently of one another at each occurrence, are selected from hydrogen and any organic or organometallic groups. The materials are prepared with high solid state quantum yield and high thermal stability.

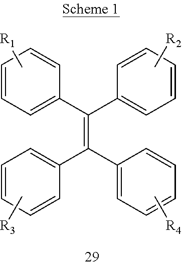

In one embodiment, oligomers and macromolecules with TPE moieties 30 and 31 in the structure are prepared by the same method as shown in Scheme 2:

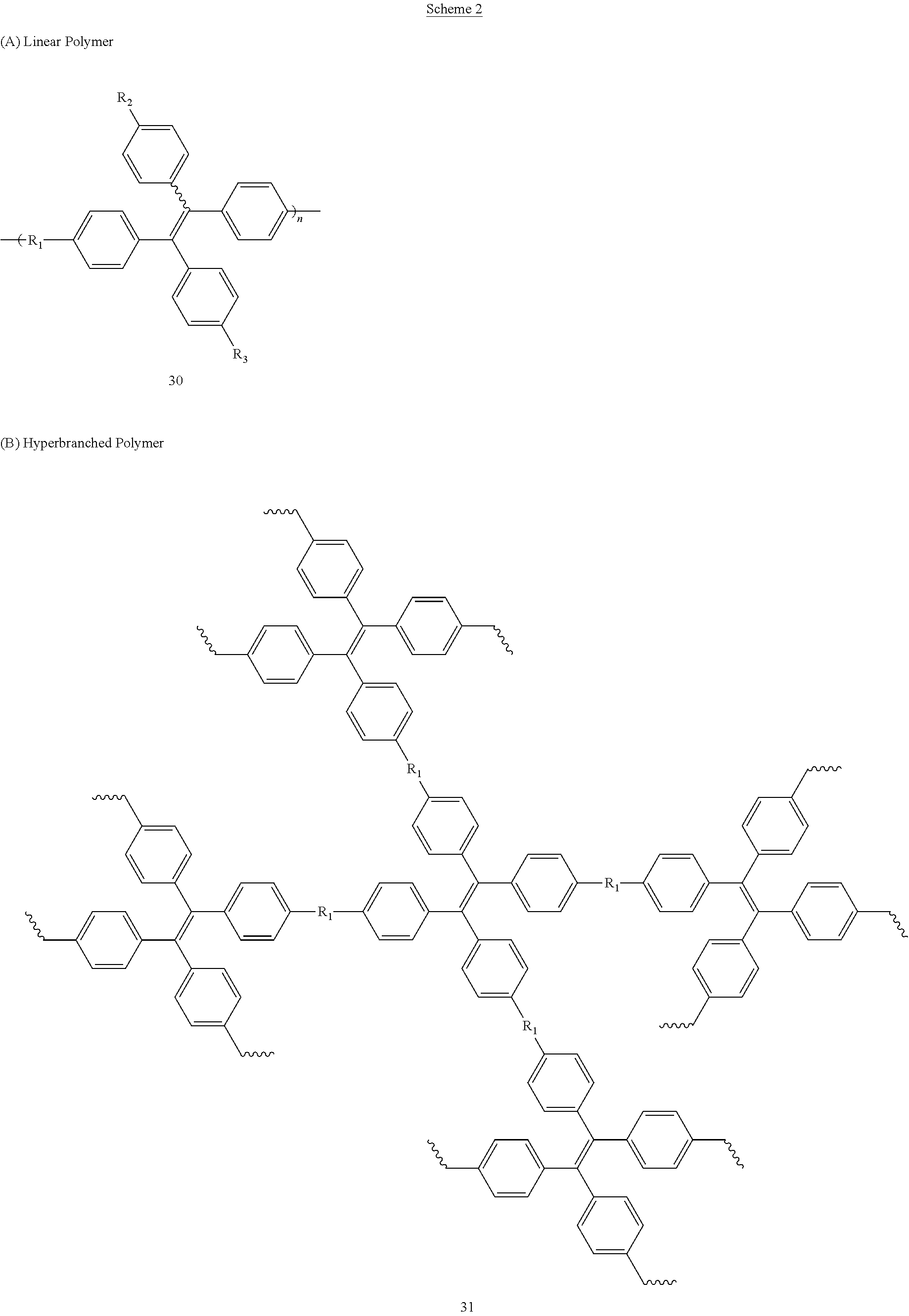

R₁, R₂, R₃, and R₄ in the molecular structures above may be each independently any compound, including organic or organometallic functionalities. Different TPE-derivatives can be obtained by changing the reactants.

The method can be applied to any kind of materials including simple organic small molecules, organometallic compounds or even macromolecules. The method employs a simple way to increase the luminescence of dyes in their solid states. The reagents or reactants can be obtained from commercial suppliers or prepared by simple organic reactions.

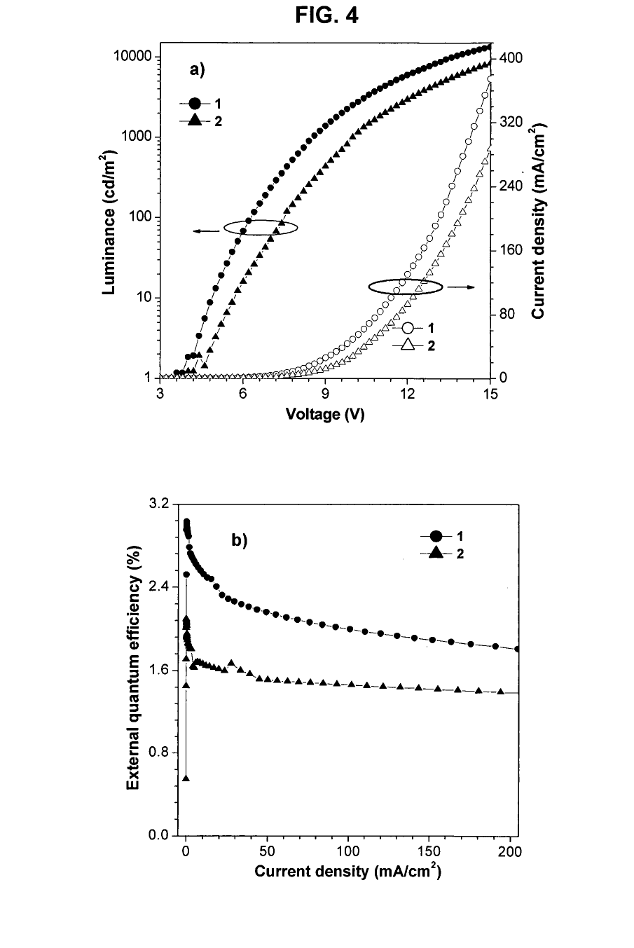

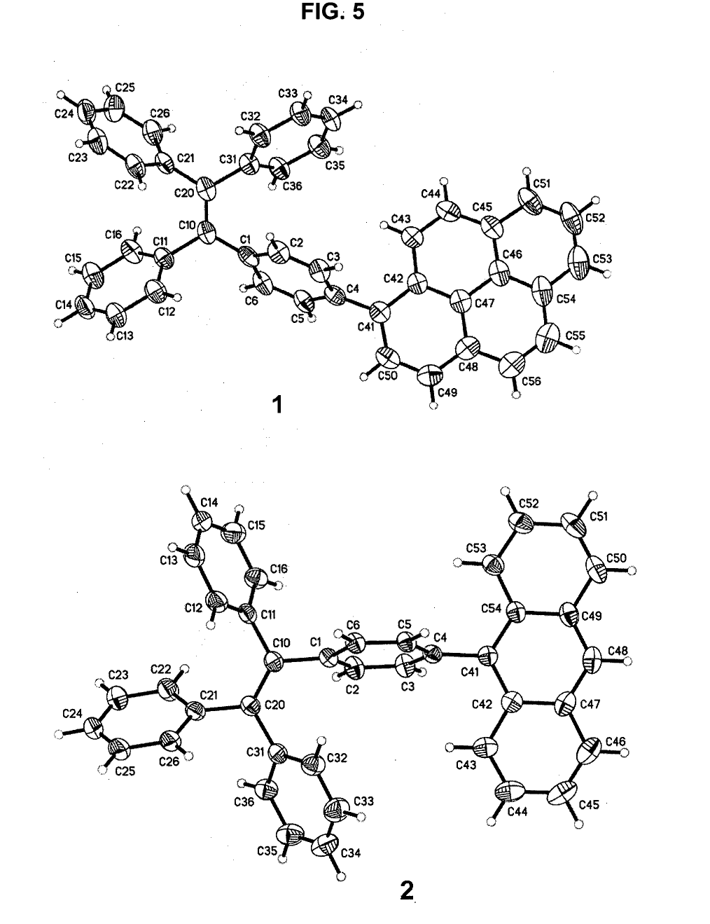

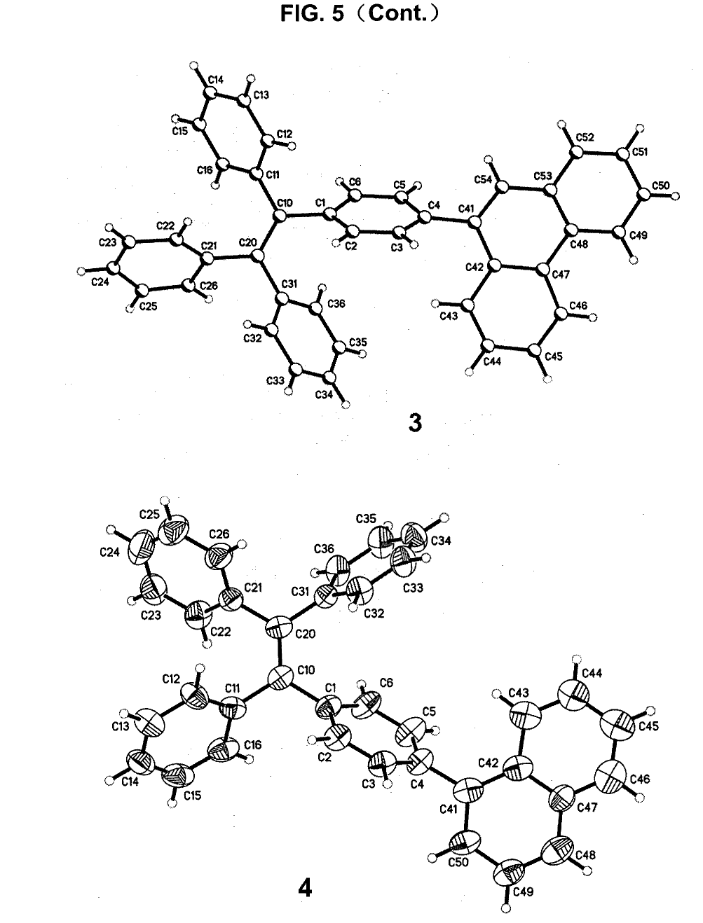

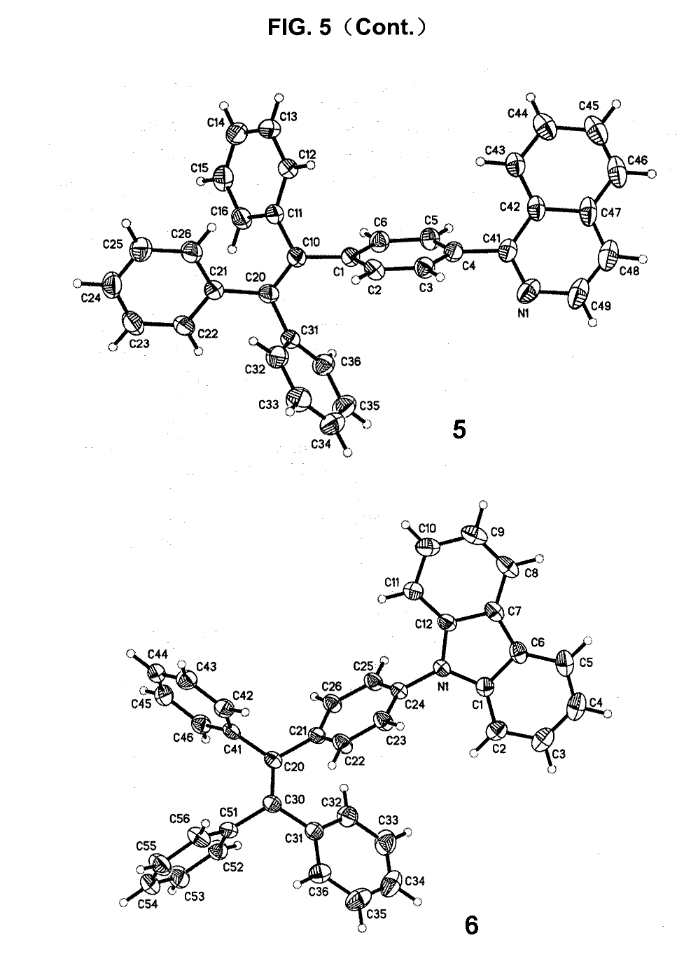

Examples of the method are shown in Chart 1 to Chart 6. As shown in Chart 1, all the desirable products are obtained from moderate to high yields (63-85%). Single crystals of the compounds are grown from their methanol/dichloromethane solutions and analyzed by X-ray diffraction crystallography. The crystal structures of the compounds are shown in FIG. 5 and their crystal analysis data are given in Tables 3 and 4.

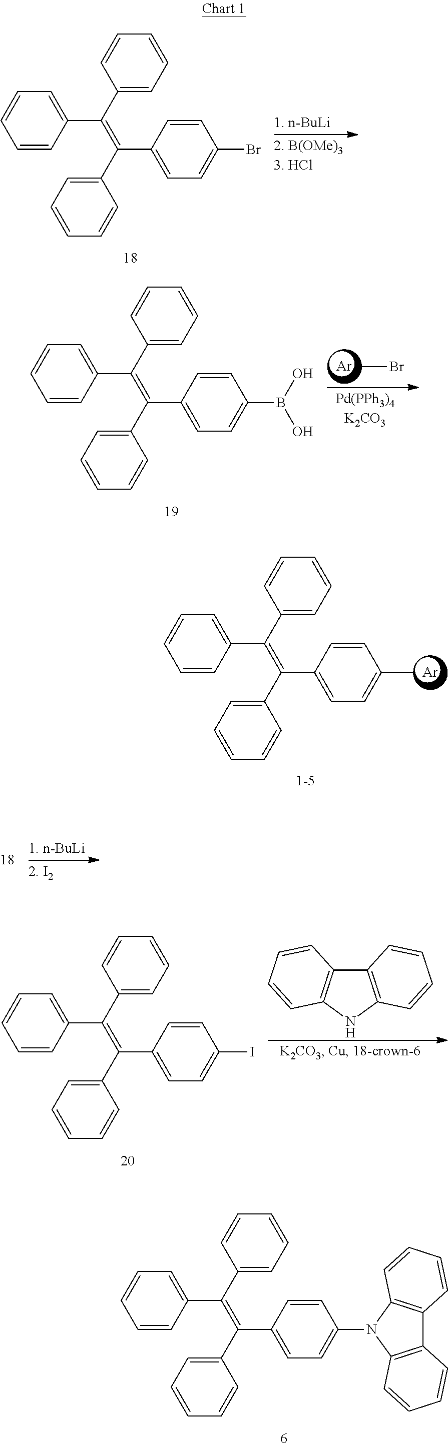

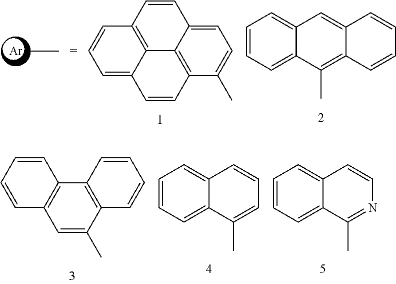

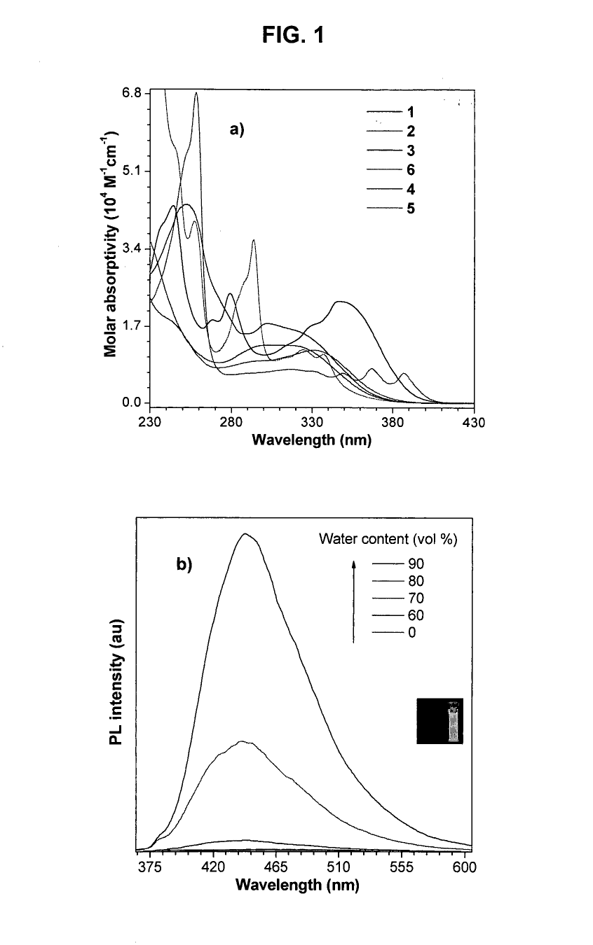

FIG. 1 (a) shows the absorption spectra of 1-6 in THF solutions. The spectral to profile and peak absorptivity vary strongly with the type of planar luminogenic unit. All the molecules show low fluorescence quantum yields (φ_{F}s) from 0.019-0.34% (see TABLE 1 below) when they are dissolved into THF to form a dilute solution, indicating that they are practically nonluminescent when molecularly dissolved in their good solvents.

TABLE 1
Optical properties of 1-6 in solution (Soln)^{[a]} and crystalline
(Cryst)^{[b]} and amorphous^{[c]} (Film) states

| | | |
|---|---|---|
| λ_{abs} | | |
| (nm) | λₑₘ (nm) | Φ_{F} (%) |

| | Soln | Soln | Cryst | Amor | Soln^{[d]} | Amor^{[e]} |
|---|---|---|---|---|---|---|
| 1^{[f]} | 348 | 432 | 443 | 468 | 0.34 | 100 |
| 2^{[g]} | 387 | 423 | 428 | 450 | 0.28 | 100 |
| 3 | 323 | | 444 | 481 | 0.033 | 88 |
| 4 | 321 | | 452 | 469 | 0.022 | 83 |
| 6 | 337 | | 440 | 468 | 0.045 | 100 |
| 5 | 331 | | 445 | 471 | 0.019 | 20 |

^{[a]}In THF (10 μM) solution.
^{[b]}Grown from methanol/dichloromethane mixture.
^{[c]}Film spin-coated on quartz plates.
^{[d]}Quantum yields (Φ_{F}) determined in THF solutions using 9,10-diphenylanthracene (Φ_{F} = 90% in cyclohexane) as standard.
^{[e]}Quantum yields of the amorphous film measured by integrating sphere.
^{[f]}For its pyrene parent, Φ_{F} = 32% in solution.
^{[g]}For its anthracene parent, Φ_{F} = 36% in solution.

Similar to TPE, the dye molecules become strong emitters when they are aggregated. As shown in FIG. 1 (b), the emission of 1 is intensified when a large amount of water (>70%) is added into its THF solution. The higher the water content, the stronger is the light emission. Since water is a nonsolvent of 1, the molecules must have aggregated in the aqueous mixtures with high water contents. This verifies that the PL of the molecule is enhanced by aggregate formation. Higher water content populates the aggregates, thereby boosting the light emission to a greater extent. Similar emission enhancement behaviors are also observed in 2-6, suggesting that the attachment of TPE unit to conventional luminophors has endowed the resultant molecules with a novel feature of AIE.

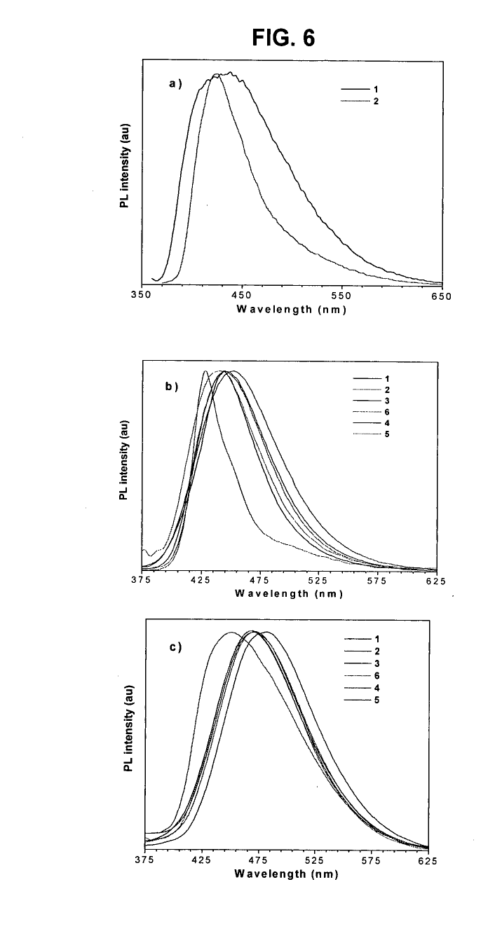

Like their aggregates suspended in aqueous media, 1-6 are highly emissive in the solid states. Upon photoexcitation, their crystals emit deep blue PL from 428 to 452 nm (FIG. 6 (b)). The crystal emissions of 1 and 2 are located at wavelengths close to those in THF solutions, indicating that the PL orginates from the same radiative decay of singlet excitons induced by photoexcitation. The spectral patterns of the amorphous films resemble those of crystals, but the emissions now move to longer wavelengths of 450 to 481 nm (FIG. 6 (c)). The quantum yields (φ_{F}s) of their amorphous films are much higher than those in solutions (Table 1). The values measured by integrating sphere reach 100% in 1, 2, and 6 which are superior than those of pyrene, anthracene, and even TPE (79.6%) in the solid state.

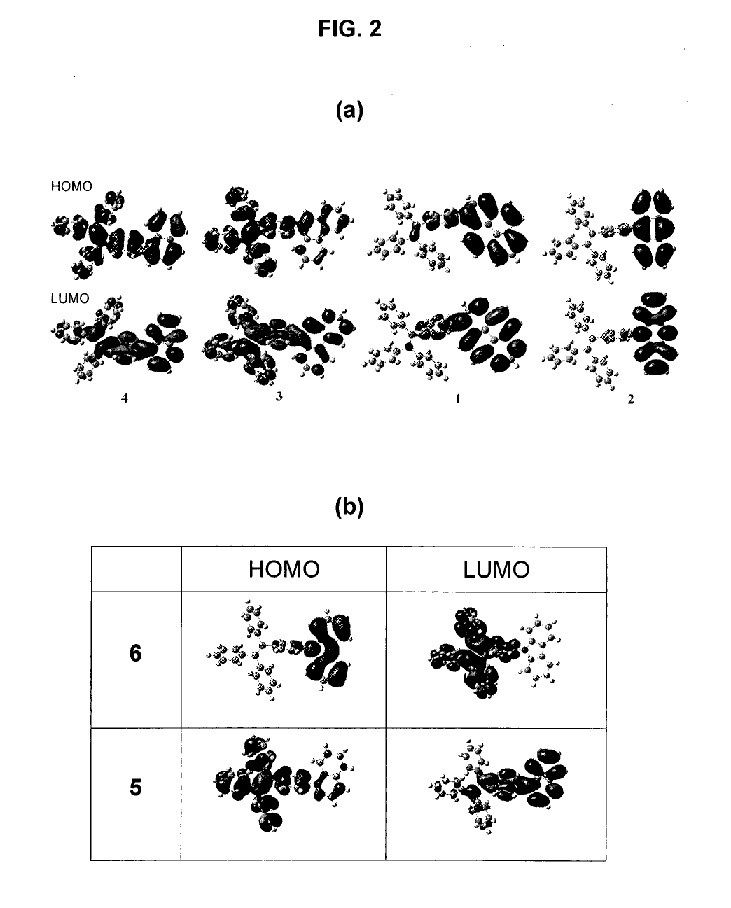

The crystal data show that all the molecules adopt highly twisted conformations in crystal states due to the existence of the propeller-like TPE moiety. The torsion angles between the planar luminophors and the directly linked phenyl rings of TPE are 66.74° (1), 75.27° (2), 58.10° (3), 78.85° (6), 51.76° (4), 52.73° (5), respectively. Compounds 2 and 6 exhibit the highest torsion angles because of the severe setric hindrance between the TPE moiety and the flat anthracene and carbazole rings. The conformations of the molecules affect strongly their HOMO and LUMO energy levels. The calculated molecular orbitals of 1, 2, 3, and 4 are displayed in FIG. 2 (a), and those of 5 and 6 are given in FIG. 2 (b).

The orbitals of 3 and 4 are dominated by the contributions from their TPE moieties and planar aromatic rings, indicating that the PL originates from the exciton decay of the whole molecules. However, TPE contributes less to the orbitals when the torsion angles become higher because of its less efficient orbital overlap and electronic communication with the planar luminogenic units. Thus, in 1 and 2, the electron densities are mainly located on the pyrene and anthracene rings, and the absorption and emission of the molecules are mainly controlled by these chromophores.

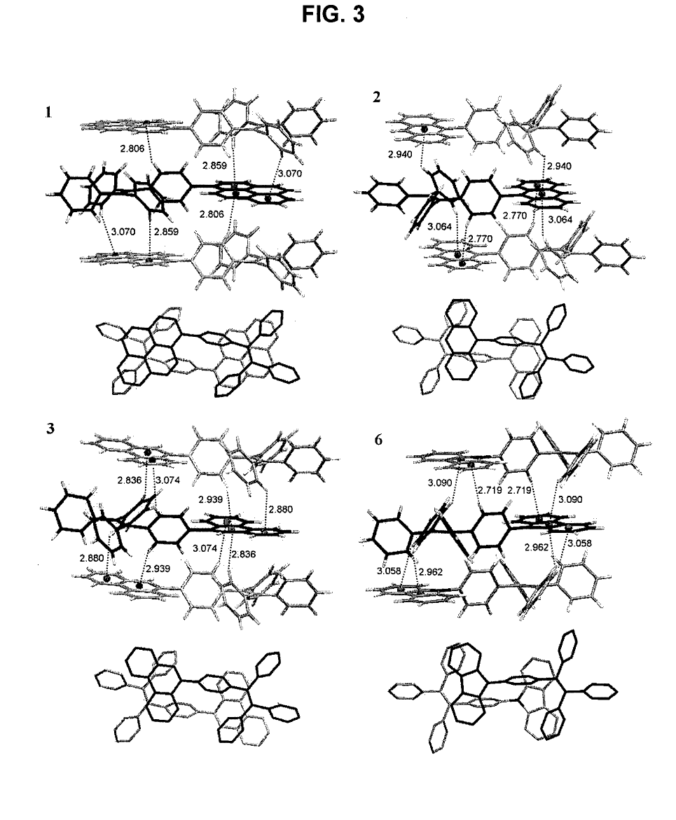

The geometric structures and packing arrangements of the compounds in the crystalline state were checked. The packing models of crystals of 1, 2, 3, and 6 resemble anchors (FIG. 3). The planar aromatic rings are situated between two TPE units, which efficiently hampers their π-π interactions and hence excimer formation. The TPE moieties are also sandwiched between two planar units. Multiple C—H . . . π hydrogen bonds with distances of 2.719-3.090 Å are formed between the hydrogen atoms of the phenyl rings in TPE moiety in one molecule and the π cloud of large planar aromatic ring in another molecule. These multiple C—H . . . π hydrogen bonds help to rigidify the molecular conformation and have locked the molecular rotations. As a result, the excited state energy consuming by the IMR process is greatly reduced, which is enabling the molecules to emit intensely in the solid state. Since there is no such constraint in the amorphous film, the TPE-Ar molecules may have adopted a more planar conformation and hence emit a redder light.

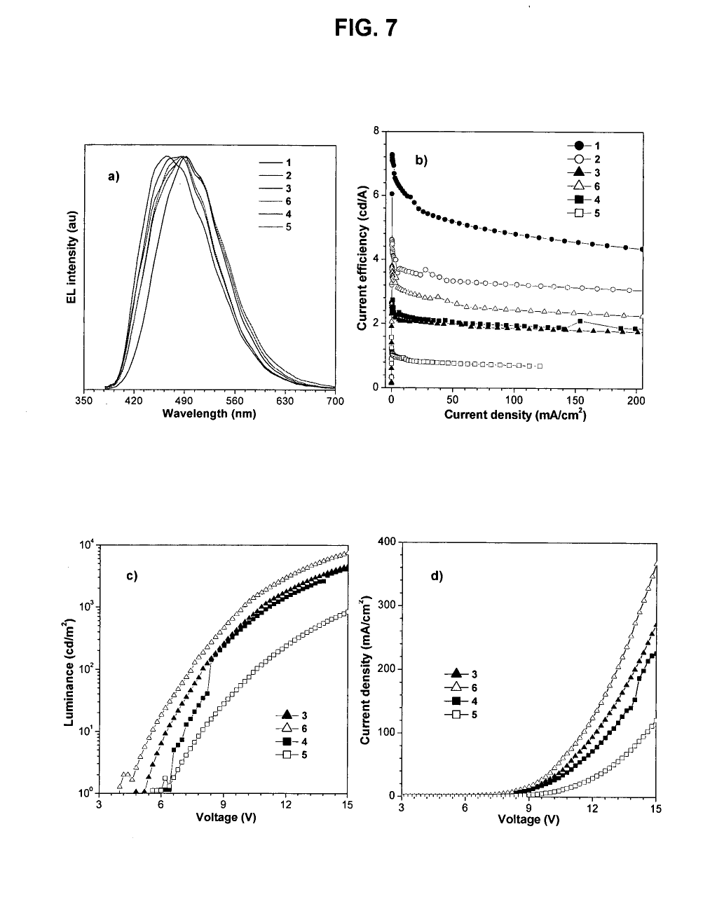

Multilayer light-emitting diodes with a configuration of ITO/NPB(60 nm)/TPE-Ar(20 nm)/TPBi(10 nm)/Alq₃(30 nm)/LiF(1 nm)/Al(100 nm) are fabricated. In these EL devices, TPE-Ar works as a light emitter, NPB functions as a hole-transport material, and TPBi and Alq₃ serve as hole-block and electron-transport materials, respectively. Also, the energy source in the EL devices is electricity from electrical socket. The EL performances of 1 and 2 are shown in FIG. 4 for instance, while others are provided in FIG. 7 and summarized in Table 2, below.

TABLE 2
EL performances of TPE-Ars.

| | EL | Vₒₙ | Lₘₐₓ | PEₘₐₓ | CEₘₐₓ | EQEₘₐₓ |
|---|---|---|---|---|---|---|
| | (nm) | (V) | (cd/m²) | (lm/W) | (cd/A) | (%) |
| 1 | 492 | 3.6 | 13400 | 5.6 | 7.3 | 3.0 |
| 2 | 488 | 4.0 | 8410 | 3.7 | 4.6 | 2.1 |
| 3 | 486 | 5.2 | 4600 | 1.6 | 2.7 | 1.4 |
| 4 | 480 | 6.0 | 4120 | 1.1 | 2.4 | 1.3 |
| 5 | 488 | 5.6 | 840 | 0.6 | 1.1 | 0.5 |
| 6 | 484 | 4.0 | 7508 | 2.7 | 3.8 | 1.8 |

Abbreviations: Vₒₙ = turn-on voltage at 1 cd/m², Lₘₐₓ = maximum luminance, PEₘₐₓ, CEₘₐₓ, and EQEₘₐₓ = maximum power, current, and external quantum efficiencies, respectively.

TABLE 3
Crystal data and intensity collection parameters for 5, 6, and 4.

| | 5 | 6 | 4 |
|---|---|---|---|
| Empirical formula | C₃₅H₂₅N | C₃₈H₂₇N | C₃₆H₂₆ |
| Mol wt | 459.56 | 497.61 | 458.57 |
| Crystal dimensions, | 0.40 × 0.10 × | 0.40 × 0.16 × | 0.25 × 0.12 × |
| mm | 0.08 | 0.14 | 0.10 |
| Crystal system | Triclinic | Triclinic | Monoclinic |
| Space group | P-1 | P-1 | P2(1)/n |
| a, Å | 9.3196(5) | 9.4375(8) | 9.1633(8) |
| b, Å | 9.3671(5) | 9.5683(7) | 28.882(3) |
| c, Å | 14.8298(8) | 15.5001(12) | 19.778(2) |
| α, deg | 88.746(4) | 83.076(6) | 90 |
| β, deg | 86.170(4) | 81.063(7) | 101.303(10) |
| γ, deg | 75.274(4) | 85.554(7) | 90 |
| V, Å³ | 1249.27(12) | 1370.13(19) | 5133.0(9) |
| Z | 2 | 2 | 8 |
| D_{calcd.}, g cm³ | 1.222 | 1.206 | 1.187 |
| F₀₀₀ | 484 | 1376 | 1936 |
| Temp, (K) | 173(2) | 173(2) | 173(2) |
| Radation (λ), Å | 1.54178 | 1.54178 | 1.54178 |
| μ (Mo Kα) mm⁻¹ | 0.534 | 0.527 | 0.507 |
| 2θₘₐₓ, deg | 66.5 (95.3%) | 66.5 (97.3%) | 66.5 (88.8%) |
| (completeness) | | | |
| No. of collected reflns. | 6612 | 7122 | 12975 |
| No. of unique reflns. | 4286 (0.0283) | 4816 (0.0291) | 8184 (0.0621) |
| (Rᵢₙₜ) | | | |
| Data/restraints/ | 4286/119/389 | 4816/0/352 | 8184/120/625 |
| parameters | | | |
| R₁, wR₂ | 0.0453, 0.1161 | 0.0404, 0.1066 | 0.0752, 0.1445 |
| [obs I > 2σ (I)] | | | |
| R₁, wR₂ (all data) | 0.0637, 0.1247 | 0.0488, 0.1105 | 0.1884, 0.1753 |
| Residual peak/hole | 0.150/−0.132 | 0.189/−0.185 | 0.278/−0.204 |
| e.Å⁻³ | | | |
| Transmission ratio | 1.00/0.70 | 1.00/0.85 | 1.00/0.55 |
| Goodness-of-fit on F² | 1.025 | 1.035 | 1.017 |

TABLE 4
Crystal data and intensity collection parameters for 3, 2, and 1.

| | 3 | 2 | 1 |
|---|---|---|---|
| Empirical formula | C₄₀H₂₈ | C₄₀H₂₈ | C₄₂H₂₈ |
| Mol wt | 508.62 | 508.62 | 532.64 |
| Crystal dimensions, | 0.30 × 0.28 × | 0.40 × 0.18 × | 0.28 × 0.20 × |
| mm | 0.04 | 0.04 | 0.04 |
| Crystal system | Monoclinic | Triclinic | Monoclinic |
| Space group | P2(1)/c | P-1 | I2/a |
| a, Å | 17.2911(10) | 9.4850(8) | 16.7865(17) |
| b, Å | 9.0613(5) | 9.6830(9) | 9.2933(6) |
| c, Å | 18.1015(10) | 15.5796(14) | 36.414(5) |
| α, deg | 90 | 79.592(8) | 90 |
| β, deg | 94.720(6) | 83.901(7) | 91.020(12) |
| γ, deg | 90 | 85.662(7) | 90 |
| V, Å³ | 2826.5(3) | 1397.0(2) | 5679.8(10) |
| Z | 4 | 2 | 8 |
| D_{calcd.}, g cm³ | 1.195 | 1.209 | 1.246 |
| F₀₀₀ | 1072 | 536 | 2240 |
| Temp, (K) | 100(2) | 173(2) | 173(2) |
| Radation (λ), Å | 1.54178 | 1.54178 | 1.54178 |
| μ (Mo Kα) mm⁻¹ | 0.511 | 0.517 | 0.534 |
| 2θₘₐₓ, deg | 66.5 (90.1%) | 66.5 (97.3%) | 66.5 (94.4%) |
| (completeness) | | | |
| No. of collected reflns. | 7739 | 7906 | 7833 |
| No. of unique reflns. | 4572 (0.1003) | 4897 (0.0363) | 4814 (0.0657) |
| (Rᵢₙₜ) | | | |
| Data/restraints/ | 4572/0/356 | 4897/0/361 | 4814/0/379 |
| parameters | | | |
| R₁, wR₂ | 0.0785, 0.0949 | 0.0378, 0.0823 | 0.0553, 0.0946 |
| [obs I > 2σ (I)] | | | |
| R₁, wR₂ (all data) | 0.1703, 0.1090 | 0.0573, 0.0866 | 0.1435, 0.1135 |
| Residual peak/hole | 0.220/−0.205 | 0.128/−0.151 | 0.130/−0.168 |
| e.Å⁻³ | | | |
| Transmission ratio | 1.00/0.79 | 1.00/0.87 | 1.00/0.64 |
| Goodness-of-fit on F² | 1.012 | 1.015 | 1.008 |

All the devices emit sky blue lights in the range from 480 to 492 nm (FIG. 7(a)), which are slightly red-shifted from the PL of their amorphous films. A device based on 1 shows the best performance. The device is turned on at a low bias of 3.6 V, and radiates brilliantly with luminance up to 13,400 cd/cm² at 15 V. The maximum current and external quantum efficiencies of the device reach 7.3 cd/A and 3.0%, respectively. Although the device configuration is yet to be optimized, the EL data are close to those attained by commercial pyrene-based luminophors (Adv. Funct. Mat. 2008, 18, 67), which is clearly demonstrative of the high potential of TPE-Ars as active layers in the construction of efficient EL devices.

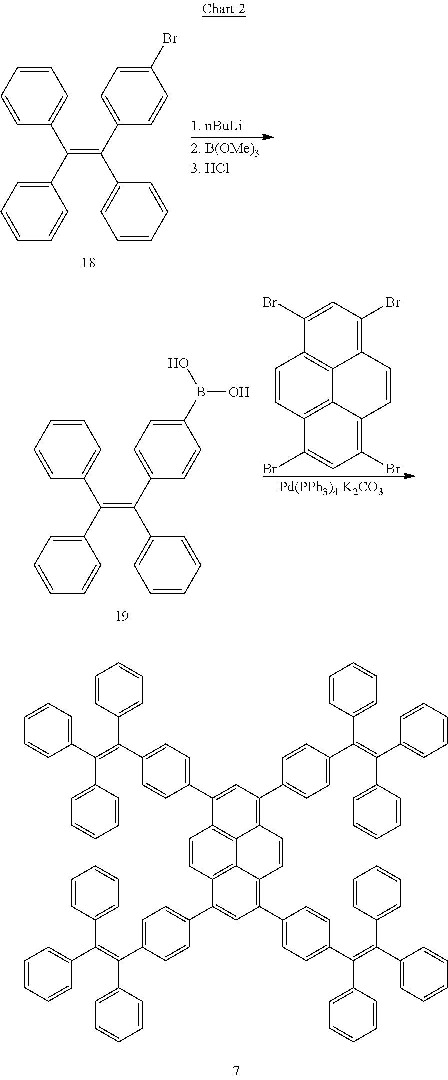

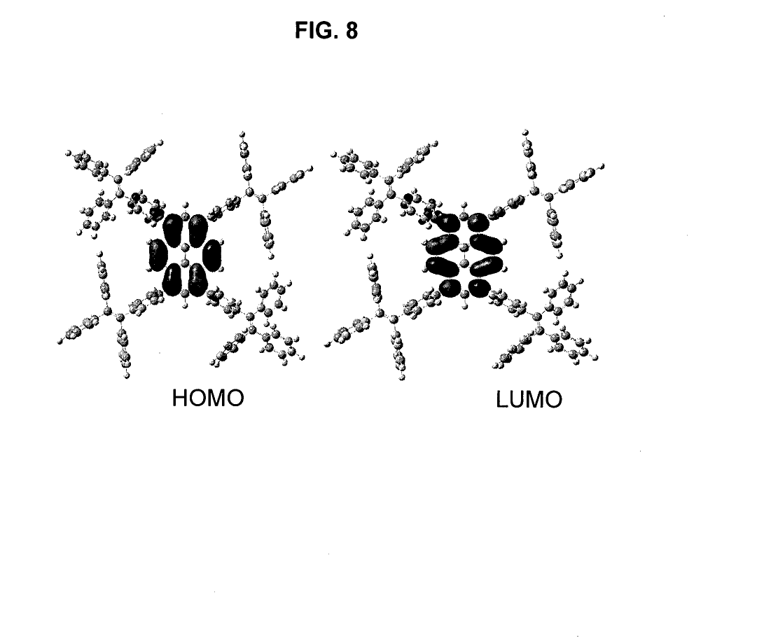

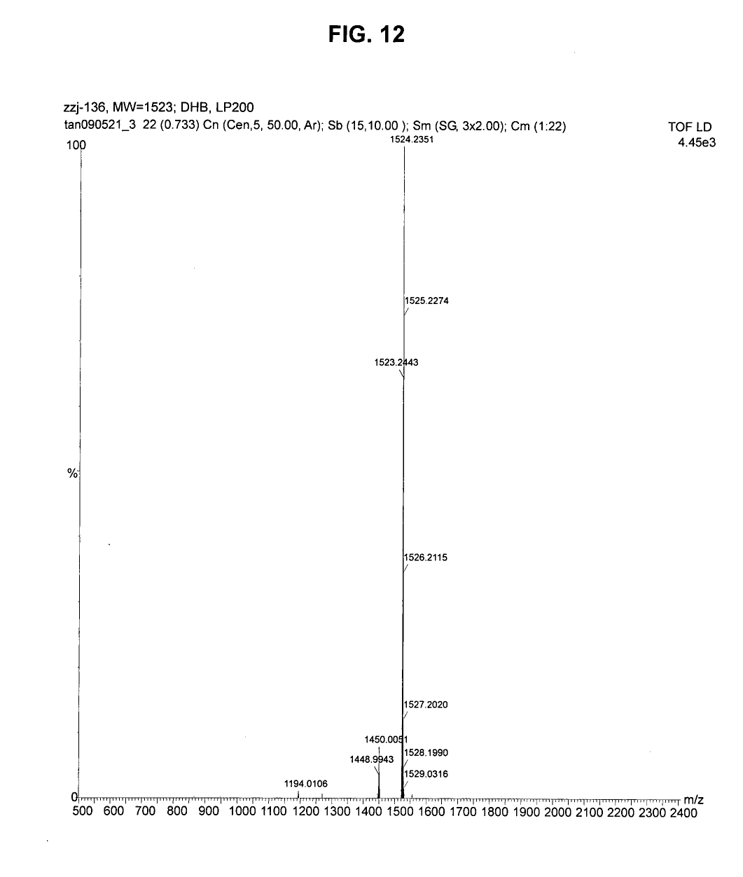

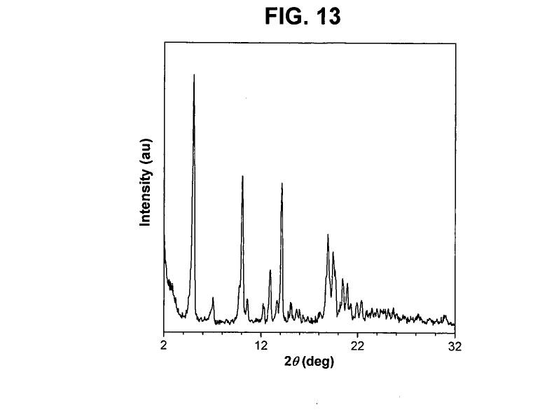

Chart 2 shows the synthesis of compound 7, and the structure of 7 is characterized by MALDI-TOF mass spectroscopy (FIG. 12). The as-prepared product is crystalline, as revealed by the XRD diffractogram (FIG. 13). Its molecular structure is optimized by the semiempirical PM3 method, in which the peripheral phenyl rings are arranged in a propeller shape. FIG. 8 shows the molecular orbital to amplitude plots of HOMO and LUMO of 7. They are mainly dominated by orbitals from the pyrene ring. The phenyl rings linked at the 1, 3, 6, 8-positions of pyrene have slight contribution to both energy levels, while the others have no contribution. This suggests that the emission of 7 mainly originates from the excited states of the central pyrene core.

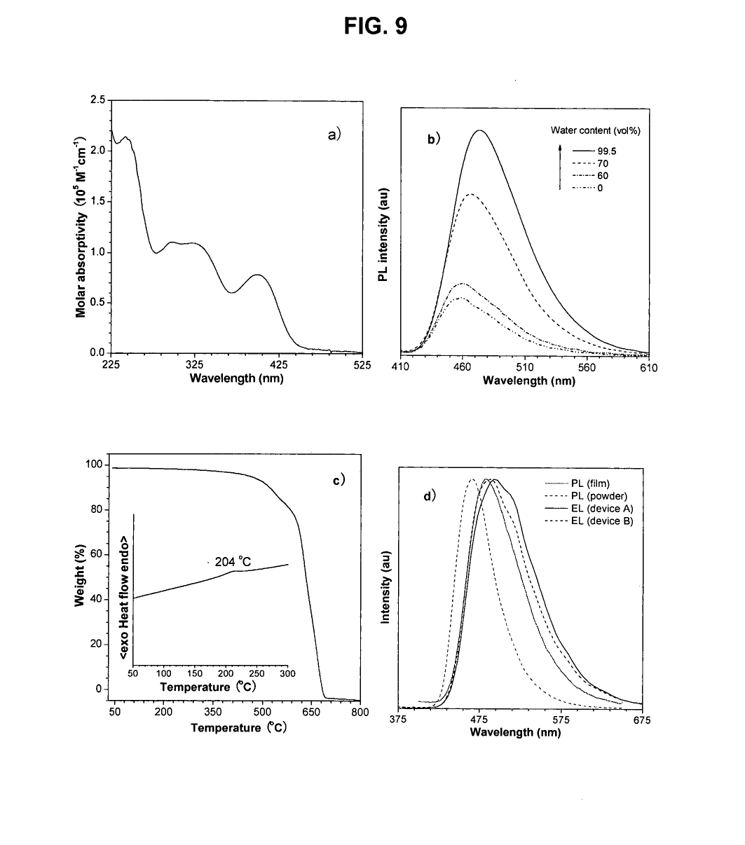

The absorption maximum of 7 is located at 398 nm, corresponding to the π-π* transition of the pyrene core with a certain extension (FIG. 9 (a)). From the onset of absorption, the energy band gap is calculated to be 2.8 eV. The emission of 7 in dilute THF solution is at 462 nm. The fluorescence quantum yield (φ_{F}) is 9.5% using 9,10-diphenylanthracene as standard (φ_{F}=90% in cyclohexane).

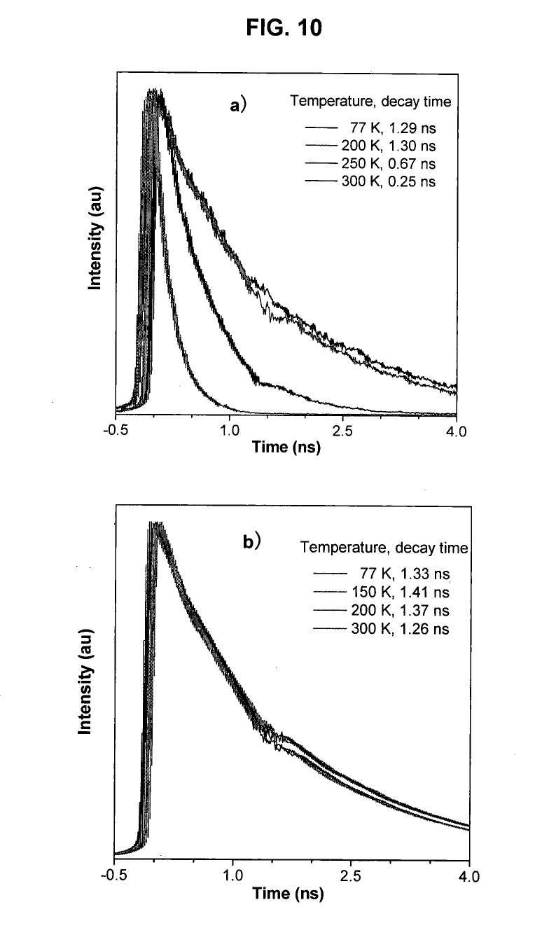

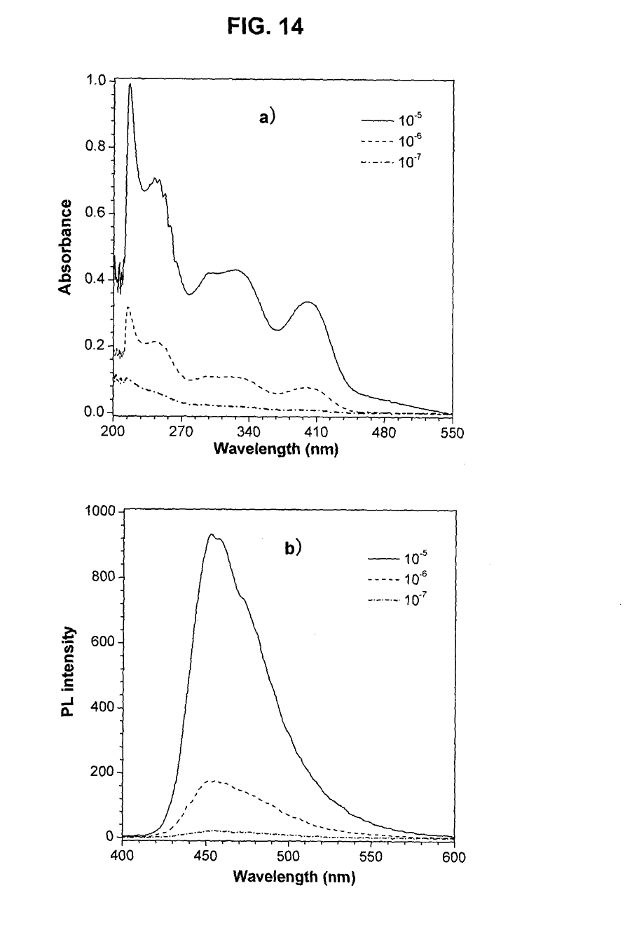

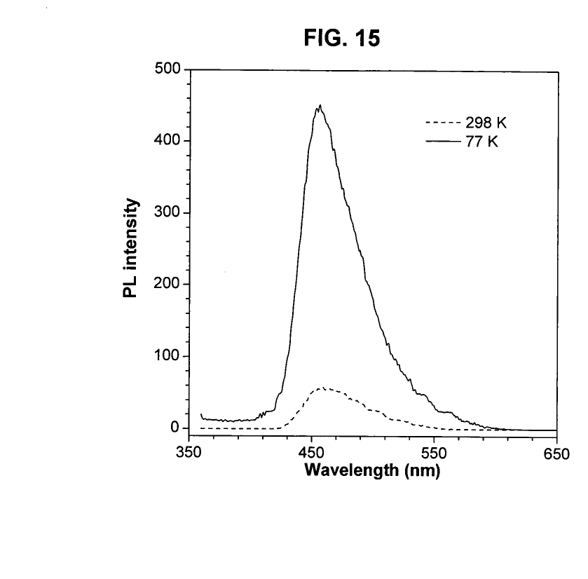

Increasing the concentration of 7 in solution leads to enhancement in intensity of absorption and emission without changes in peak positions (FIG. 14). Decreasing the temperature of 7 in the THF solution results in an emission enhancement with little change in the emission wavelength (FIG. 15). The singlet excited state of 7 rapidly decays single-exponentially with a short lifetime of 0.25 ns at 300K in solution, and the lifetime becomes longer when the temperature is cooled down and reaches 1.29 ns at 77 K (FIG. 10 (a)). This phenomenon suggests that the free rotation of phenyl blades as well as molecular motions that consume the excited energy of the molecules are frozen at low temperatures, resulting in emission enhancement.

Addition of a large amount of nonsolvent, such as water into its THF solution, has aggregated the molecules and also restricted the intramolecular rotation, which has imparted the solution with a stronger emission. The emission remains almost unchanged when up to 60% water is added to the THF solution but starts to increase afterwards accompanying with a slight red-shift in the emission maximum (FIG. 9 (b)).

The emission of crystalline powders of 7 is at 465 nm, which is close to that in pure solution, indicating that the emission originates from 7 monomers. The amorphous film emits at 483 nm (FIG. 9 (d)), which is red-shifted by 18 nm compared to that of crystalline powders. The blue-shifted emission in the crystalline state is not an isolated case observed in 7 but has been found in other TPE derivatives, due to the conformation twisting in the crystal packing process.

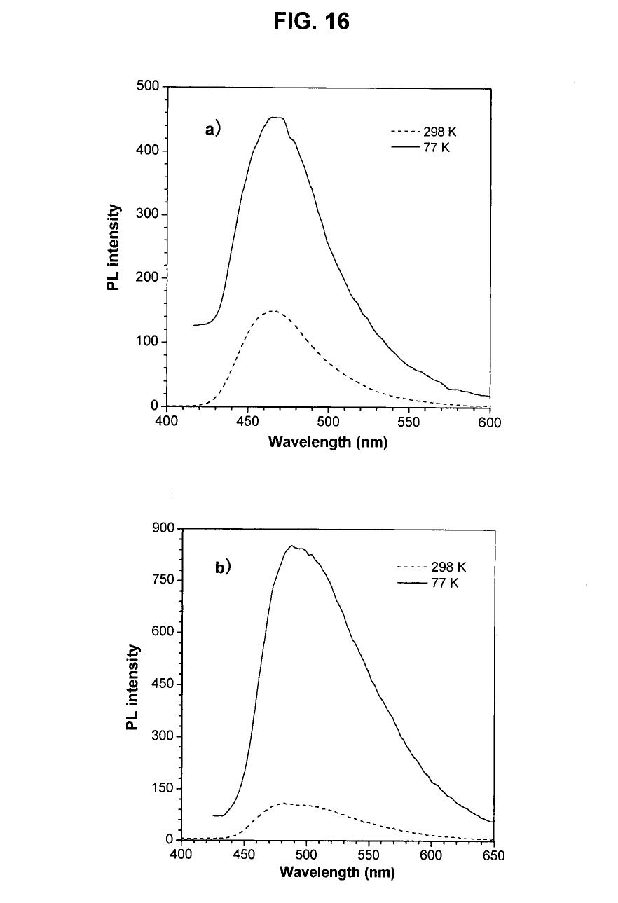

The emissions in both crystalline and amorphous states became stronger when the temperatures were lowered (FIG. 16). The crystalline powders undergo single-exponential decay from the singlet excited to ground states. The lifetime is 1.26 ns, which is much longer than that in solution at 300K, and it varies little at low temperatures (FIG. 10 (b)). This suggests that the twisted molecular conformation in crystalline state has restricted the molecular rotation efficiently. The absolute solid φ_{F} of 7 is 70% as measured from its amorphous film by integrating sphere.

The thermal properties of 7 are examined by DSC and TGA analyses. The glass-transition (T_{g}) and onset decomposition temperatures are 204° C. and 460° C., respectively (FIG. 9 (c)). Although the molecular weight of 7 reaches 1,524 g/mol, its good thermal stability ensures that it can be vacuum sublimed for thin film deposition in a vacuum condition of 3−7×10⁻⁷ Torr at −200° C. without degradation. The HOMO and LUMO energy levels of 7 are measured by cyclic voltammetry. The HOMO derived from their onset potential of oxidation is located at 5.4 eV, while the LUMO calculated by subtraction of the optical band gap energy from the HOMO value is 2.6 eV.

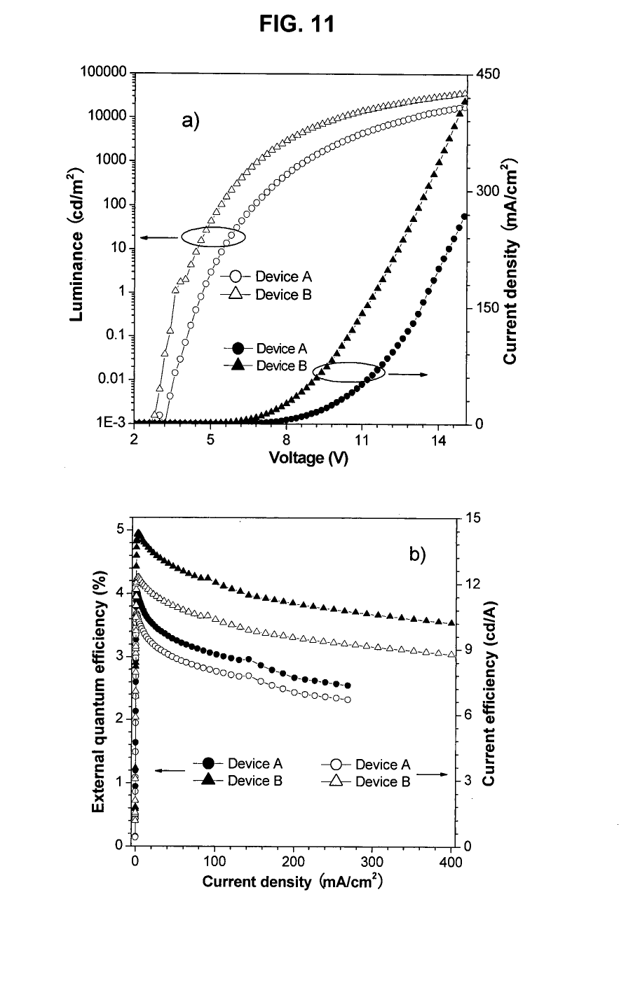

Multilayer EL devices with configurations of ITO/NPB(60 nm)/7(40 or 26 nm)/TPBi(20 nm)/LiF(1 nm)/Al(100 nm) (Device A and B) are fabricated, which give a sky blue EL at −490 nm (FIG. 9 (d)). The EL spectra are slightly red-shifted from the PL spectrum of the amorphous film. The 7-based devices enjoy good spectra stability and no obvious change in the EL spectrum when the voltage is raised up to 15 V. FIG. 11 shows the performances of devices based on 7. Device A shows a low turn-on voltage (4.7 V) and emits brilliantly (luminance=18,000 cd/m² at 15 V). The maximum current, power, and external quantum (EQEₘₐₓ) efficiencies attained by the device are 10.6 cd/A, 5.8 lm/W, and 4.04%, respectively. Even better performance is observed in device B. Compound 7 starts to emit at a lower voltage of 3.6 V and at the same voltage, the luminance reaches 36300 cd/m². The EQEₘₐₓ is 4.95% at 6 V, approaching the limit of the possible. The efficiencies remain reasonably high at high current density. For example, the efficiency is 3.5% in device B even at a high current density of 415 mA/cm². These results, although preliminary, suggest that 7 is a promising luminophor in the fabrication of OLEDs.

Table 6 summarizes the EL properties of 7. The EL from a diode (Device C) of Alg₃, a widely studied EL luminophor, is also given for comparison. Clearly, the OLEDs fabricated from 7 show much better performances than that based on Alq₃. Compared with most pyrene-containing materials, the TPE-substituted pyrene shows superior properties such as high T_{g}, solid PL efficiency, and device performance. Opposed to most pyrene-based luminophors that are highly crystalline and nonemissive in the solids states, the TPE units in 7 not only suppress the excimer formation but also increase the solid state emission via the restriction of intramolecular rotation. Using AIE molecules to modify convenient planar luminophors that suffer from emission quenching in the solid state is a new and practicable strategy to develop efficient luminescent materials.

TABLE 6
EL performances of 7 and Alq₃.

| | EL | Vₒₙ | Lₘₐₓ | PE | CE | EQEₘₐₓ |
|---|---|---|---|---|---|---|
| Device | (nm) | (V) | (cd/m²) | (lm/W) | (cd/A) | (%) |
| A | 492 | 4.7 | 18000 | 5 | 10.6 | 4.04 |
| B | 488 | 3.6 | 36300 | 7 | 12.3 | 4.95 |
| C | 520 | 3.5 | 27600 | 2.7 | 5.3 | 1.6 |

^{a} Device configuration = ITO/NPB (60 nm)/7 (40 or 26 nm)/TPBi (20 nm)/LiF (1 nm)/Al (100 nm) (Device A and B) and ITO/NPB (60 nm)/Alq₃ (40 nm)/TPBi (20 nm)/LiF (1 nm)/Al (100 nm) (Device C).
Abbreviations: Vₒₙ = turn-on voltage at 1 cd/m², Lₘₐₓ = maximum luminance, PE and CE = power and current efficiencies at 100 cd/m², EQEₘₐₓ = maximum external quantum efficiency.

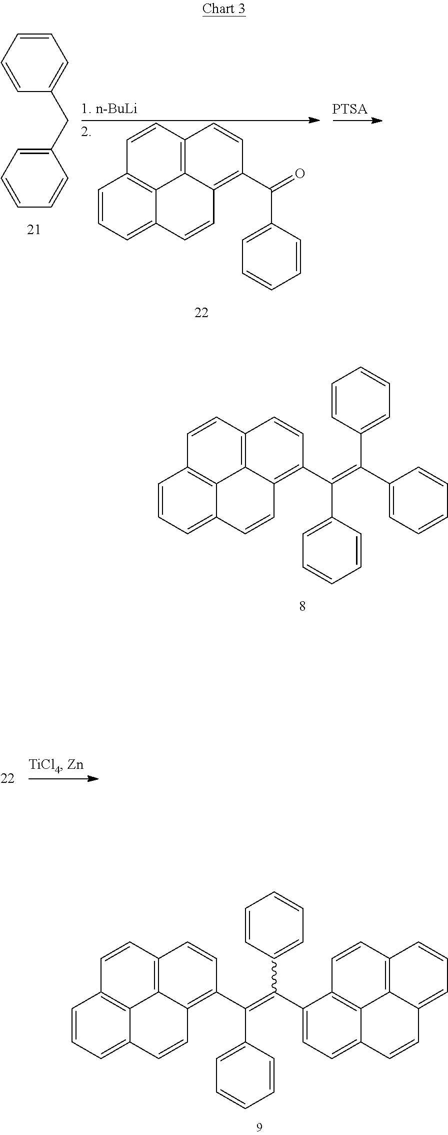

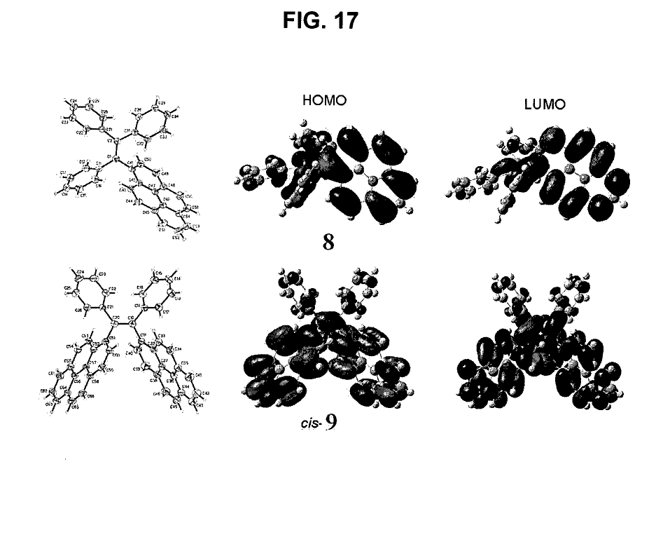

Chart 3 illustrates the synthetic routes to the pyrene-substituted ethenes. Single crystals of TPPyE were grown from its hexane/dichloromethane solution and analyzed by X-ray diffraction crystallography. Both crystals of cis- and trans-9 were obtained under the same conditions. However, only crystals of the cis-9 was desirably isolated by a very slow evaporation of its chloroform solution. The crystal structures and B3LYP/6-31G*-calculated molecular orbital amplitude plots of HOMO and LUMO levels of 8 and cis-9 are shown in FIG. 17, while the crystal data are provided in Table 9.

TABLE 9
Crystal data and intensity collection parameters for 8 and cis-9.

| | 8 | cis-9 |
|---|---|---|
| Empirical formula | C₃₆H₂₄ | C₄₆H₂₈•CHCl₃ |
| Mol wt | 456.55 | 700.05 |
| Crystal dimensions, | 0.25 × 0.20 × | 0.25 × 0.20 × |
| mm | 0.13 | 0.18 |
| Crystal system | Triclinic | Triclinic |
| Space group | P-1 | P-1 |
| a, Å | 9.5346(6) | 8.7695(6) |
| b, Å | 9.5932(6) | 13.3414(11) |
| c, Å | 13.9476(9) | 15.9829(11) |
| α, deg | 96.674(5) | 77.119(8) |
| β, deg | 105.479(6) | 89.166(6) |
| γ, deg | 94.841(5) | 71.187(6) |
| V, Å³ | 1212.24(13) | 1722.2(2) |
| Z | 2 | 2 |
| D_{calcd.}, g cm³ | 1.251 | 1.350 |
| F₀₀₀ | 480 | 724 |
| Temp, (K) | 173(2) | 173(2) |
| Radation (λ), Å | 1.54178 | 1.54178 |
| μ (Mo Kα) mm | 0.537 | 2.667 |
| 2θₘₐₓ, deg (completeness) | 66.5(95.1%) | 66.5 (90.0%) |
| No. of collected reflns. | 6354 | 8722 |
| No. of unique reflns. | 4112 (0.0277) | 5520 (0.0649) |
| (Rᵢₙₜ) | | |
| Data/restraints/ | 4112/0/325 | 5520/0/451 |
| parameters | | |
| R₁, wR₂ [obs I > 2σ (I)] | 0.0381, 0.0960 | 0.0747, 0.1970 |
| R₁, wR₂ (all data) | 0.0476, 0.0994 | 0.0875, 0.2066 |
| Residual peak/hole | 0.148/−0.183 | 0.515/−0.356 |
| e.Å⁻³ | | |
| Transmission ratio | 1.00/0.76 | 1.00/0.56 |
| Goodness-of-fit on F² | 1.012 | 1.076 |

Deposited Crystal Data Numbers: CCDC 755289 and 755290 for 8 and cis-9, respectively.

The electron clouds in both HOMO and LUMO levels of 8 and cis-9 are mainly located on the pyrene ring, revealing that this chromophoric unit controls predominately the absorption and emission of the molecules.

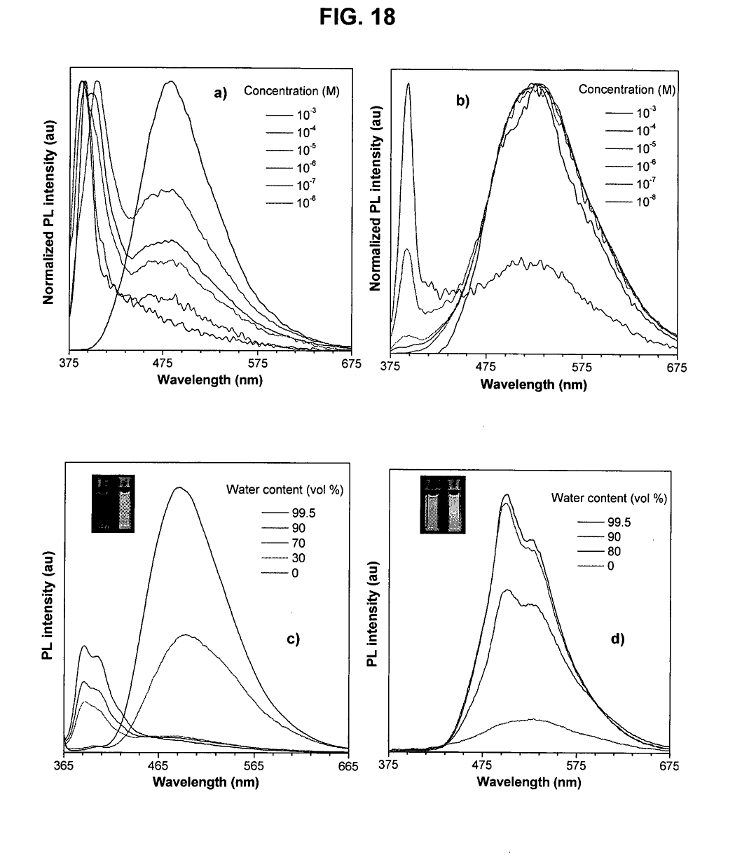

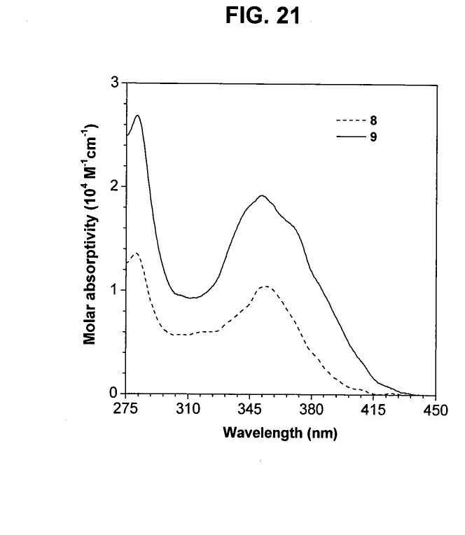

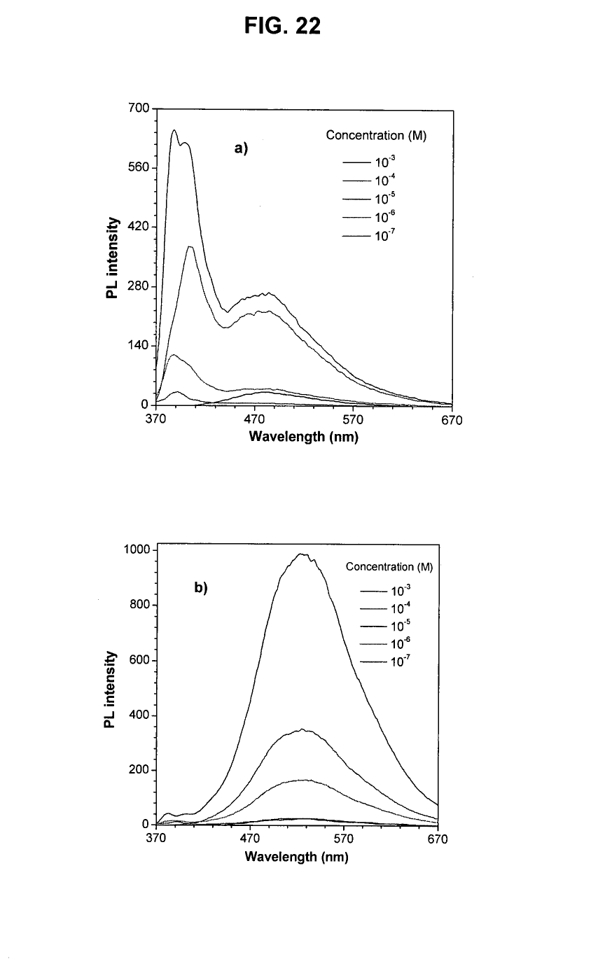

The absorption spectrum of 8 is resembled to that of 9 and both exhibit a peak maximum at ˜350 nm (FIG. 21). The absorptivity (1.9×10⁴ M⁻¹ cm⁻¹) at 353 nm in 9 is about two-fold higher than that in 8, correlating with the number of pyrene units in the molecule. The PL spectrum of a dilute THF solution (10⁻⁸ M) of 8 displays a sharp peak at 388 nm (FIG. 18 (a)). When the solution concentration is increased to 10⁻⁷ M, a new peak emerges at 483 nm. Whereas the former peak is assigned to the monomer emission of the pyrene moiety the latter one may be associated with the emission of pyrene excimers. With a progressive increase in the solution concentration, the emission at 483 nm becomes dominant albeit with a concomitant decrease in the intensity (FIG. 22 (a)). At 10⁻³ M, only emission at the longer wavelength is observed, demonstrating that it is truly originated from the pyrene excimers.

Such concentration-dependent PL spectra are also observed in 9, but at the same concentration the excimer emission is much stronger (FIG. 22 (b)). Even at a concentration as low as 10⁻⁸ M, the PL spectrum still exhibits excimer emission at 523 nm (FIG. 18 (b)). This is because 9 contains two pyrene rings, which makes excimer formation easier. This explains why its excimer emission is observed at longer wavelengths than that of 8. The fluorescence quantum yields (φ_{F}s) of 8 and 9 in dilute THF solutions (10⁻⁶ M) are 2.8% and 9.8%, respectively.

Upon photoexcitation, the crystals of 8 and cis-9 emit at 481 and 486 nm, respectively, as shown in Table 7.

TABLE 7
Optical properties of 8 and 9 in solution (Soln)^{[a]} and crystalline
(Cryst) and amorphous (Film)^{[b]} states.

| | λ_{abs} | | | | | |
|---|---|---|---|---|---|---|
| | (nm) | λₑₘ (nm) | | | Φ_{F} (%) | |
| | Soln | Soln | Cryst | Film | Soln^{[c]} | Film^{[d]} |
| 8 | 353 | (388) 483 | 481 | 484 | 2.8 | 61 |
| 9 | 352 | (391) 523 | 486^{[e]} | 503 | 9.8 | 100 |

^{[a]}In THF (10 μM) solution.
^{[b]}Film spin-coated on quartz plates.
^{[c]}Quantum yields (Φ_{F}) determined in THF solutions using 9,10-diphenylanthracene (Φ_{F} = 90% in cyclohexane) as standard.
^{[d]}Quantum yields of the amorphous film measured by integrating sphere.
^{[e]}Crystals of the cis-9.

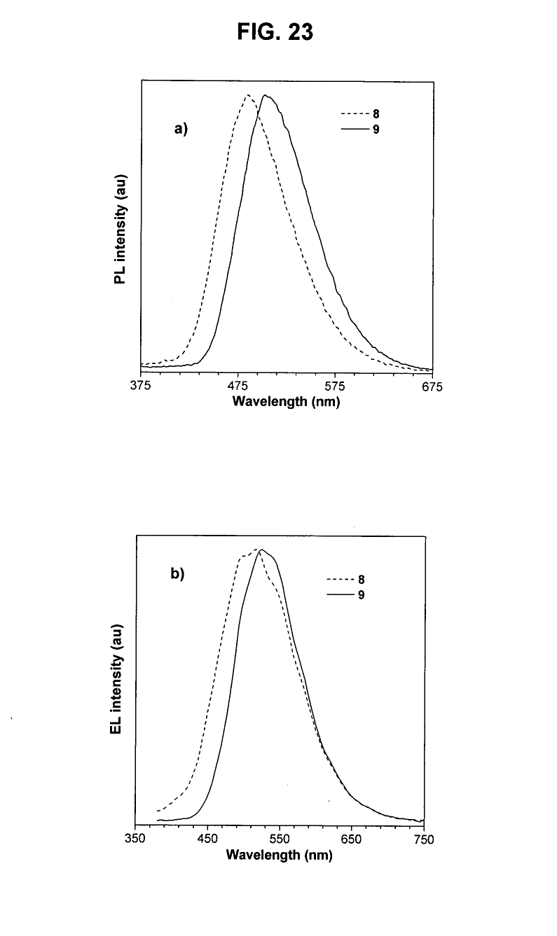

The PL of the amorphous film of 8 is found at 484 nm, which is close to those in concentrated solution and crystal state (FIG. 23 (a)), suggesting that they originate from the same emitting species with similar molecular interactions. Interestingly, the PL of the amorphous of 9 is located at 503 nm, which is 20 nm blue-shifted and 17 nm red-shifted from those in solution and crystals, respectively. The unusual blue shift observed in the crystalline phase may be attributable to the conformation twisting in the crystal packing process, during which the 9 molecules may have conformationally adjusted themselves by twisting their aromatic rings to fit into the crystalline lattices. Without such restraint, the molecules in the amorphous state may assume a more planar conformation, which enables better π-π stacking interactions and hence results in redder luminescence.

In concentrated solution, multiple excimers may be more readily formed because the molecules can adjust their conformations and positions with little constraint in order to achieve maximum intermolecular interactions. That explains why the PL is observed at the longer wavelength in the solution state. Contrary to their weak emission in dilute solutions, the φ_{F} values of the amorphous films of 8 and 9 are much higher and reach 61 and 100%, respectively. This suggests that the aggregates of both molecules emit more efficiently than their molecularly isolated species, demonstrating a novel phenomenon of aggregation-induced emission enhancement (AIEE).

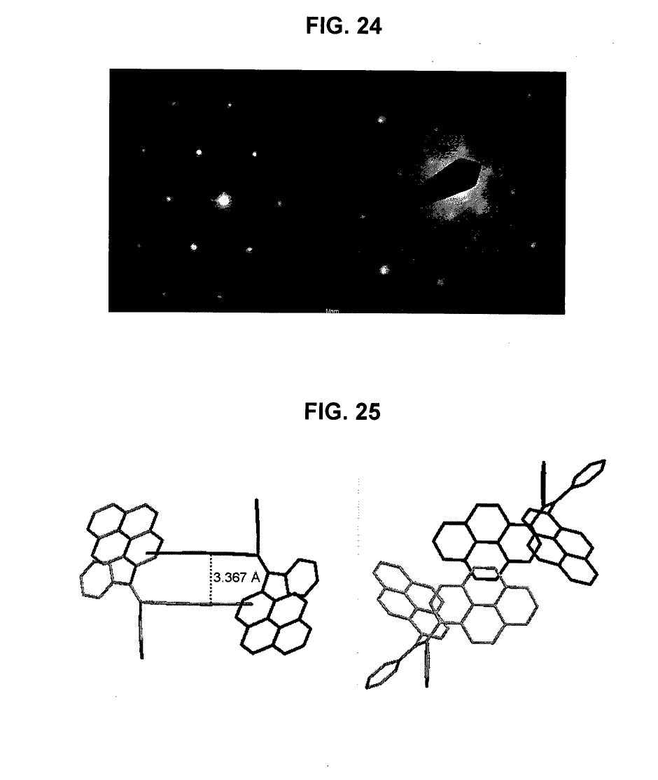

When a large amount of water is added into their THF solutions, their emissions are strengthened (FIGS. 18 (c) and 18 (d)). The monomer emission of 8 at 388 nm rises slowly with increasing water content in the THF/water mixture. At a water content of 90%, intense excimer emission is observed at 485 nm. The intensity at 99.5% water content is so strong that the monomer emission is hardly discerned. The excimer emission of 9 also becomes stronger in aqueous mixtures with higher water contents. Since 8 and 9 are not soluable in water, their molecules must be aggregated in solvent mixtures with large amounts of water. The solutions are, however, homogeneous with no precipitates, suggesting that the aggregates are of nanodimension. The ED patterns of aggregates of 8 and 9 formed in THF/water mixtures with 90% water content show many diffraction spots (FIG. 24), suggesting that they are crystalline in nature.

FIG. 19 shows the crystal packing of the compounds. The pyrene rings of two adjacent 8 molecules are stacked in a parallel fashion and about half of their surfaces (−7 carbon atoms) overlap (FIG. 19 (c)). The distance between two pyrene planes is 3.483 Å, which is shorter than the typical distance for π-π interaction (3.5 Å). Similar packing arrangements with a distance of 3.402 Å between pyrene rings of adjancent molecules are also observed in the single crystals of cis-9. This provides evidence that the PL of 8 and cis-9 in the crystal state stems from the pyrene excimers. The second pyrene ring of cis-9 is also located parallel to the pyrene blade of its neighboring molecule with a distance of 3.367 Å (FIG. 25). Although the extent of overlap is not large, it is capable of hindering their free rotations. It is surprising that the cis-9 molecules can self-assembly into a super molecular structure similar to that illustrated in FIG. 19(e) via π-π intermolecular interactions. Such head to tail connection is not formed in 8 because there is only one pyrene ring in the molecule (FIG. 19(b)). That may explain its similar emission behaviors in solution, crystalline, and amorphous states.

Instead of π-π stacking, multiple C—H . . . π hydrogen bonds with distances of 2.970 and 3.086 Å are formed between the hydrogen atoms of the phenyl rings in a 8 molecule and the π cloud of the pyrene ring in another molecule. C—H . . . π hydrogen bonds with a distance of 2.835 Å are also observed between the hydrogen atoms of the pyrene rings in one cis-9 molecule and the π cloud of the pyrene ring in another molecule. These weak but attractive forces of multiple C—H . . . π hydrogen bonds, as well as π-π interactions, help to rigidify the molecular conformation and lock the molecular rotations. As a result, the excited energy consumption by the IMR process is reduced greatly, which enables the molecules to emit intensely in the solid state.

Multilayer organic light-emitting diodes (OLEDs) with configurations of ITO/NPB(60 nm)/8 or 9(20 nm)/TPBi(30 nm)/LiF(1 nm)/Al(100 nm) (Device I) and ITO/NPB(60 nm)/8 or 9 (20 nm)/TPBi(10 nm)/Alq₃(30 nm)/LiF(1 nm)/Al(100 nm) (Device II) are fabricated. In these EL devices, 8 and 9 work as a light emitters, NPB functions as a hole-transport material, and TPBi and Alq₃ serve as hole-block and electron-transport materials, respectively. The performances of the devices are summarized in Table 8.

TABLE 8
EL performances of 8, 9 and Alq₃.

| | | EL | Vₒₙ | Lₘₐₓ | PEₘₐₓ | CEₘₐₓ | EQEₘₐₓ |
|---|---|---|---|---|---|---|---|
| | device | (nm) | (V) | (cd/m²) | (lm/W) | (cd/A) | (%) |
| 8 | I | 516 | 3.9 | 14,340 | 5.8 | 8.0 | 2.9 |
| 9 | I | 524 | 5.3 | 45,550 | 4.1 | 9.1 | 2.9 |
| 8 | II | 520 | 4.8 | 7,460 | 2.2 | 4.0 | 1.5 |
| 9 | II | 516 | 3.2 | 49,830 | 9.2 | 10.2 | 3.3 |
| Alq₃ | II | 532 | 3.9 | 8,490 | 2.9 | 5.4 | 1.6 |

Abbreviations: Vₒₙ = turn-on voltage at 1 cd/m², Lₘₐₓ = maximum luminance, PE_{max,} CE_{max,} and EQEₘₐₓ = maximum power, current, and external quantum efficiencies, respectively.

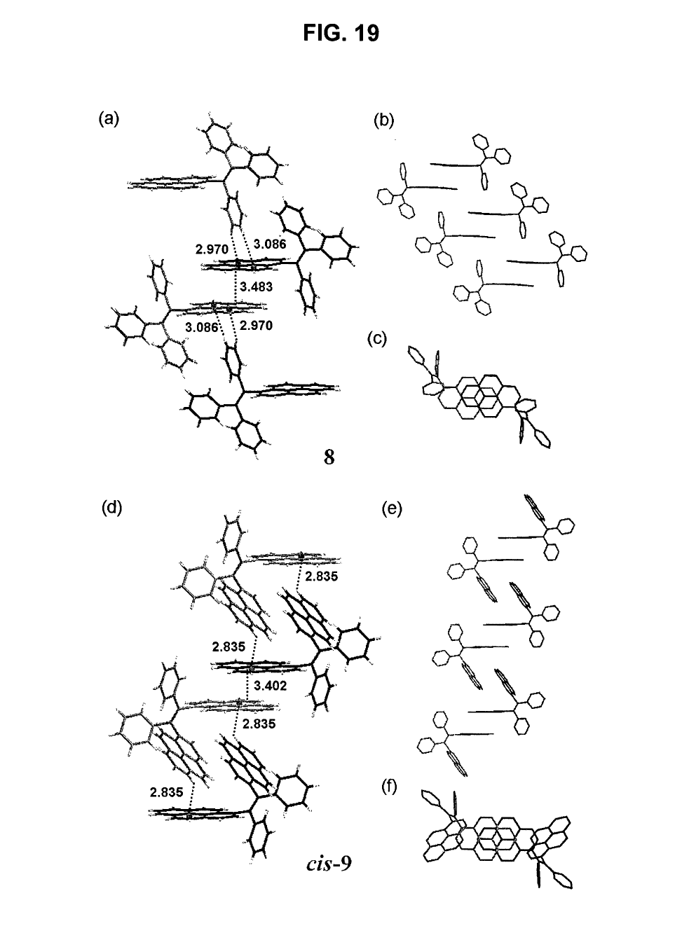

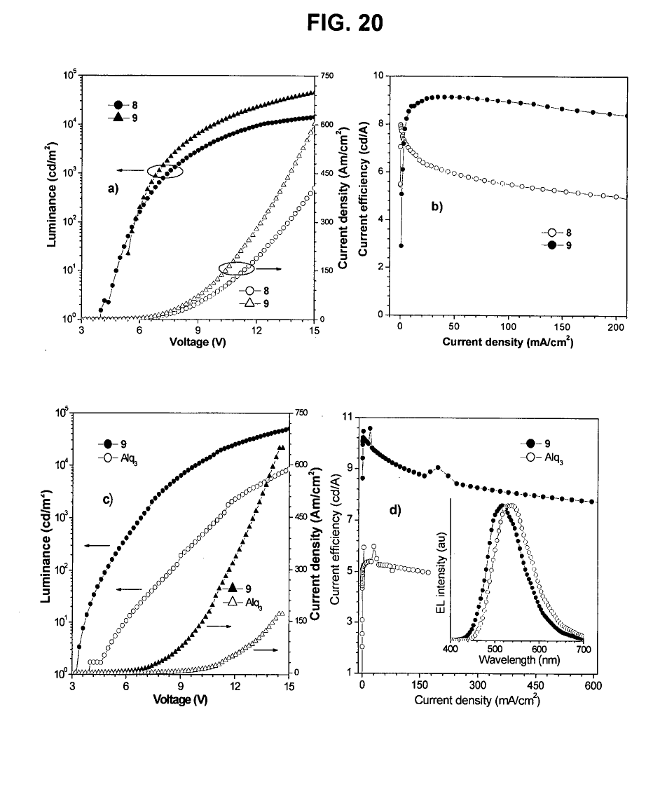

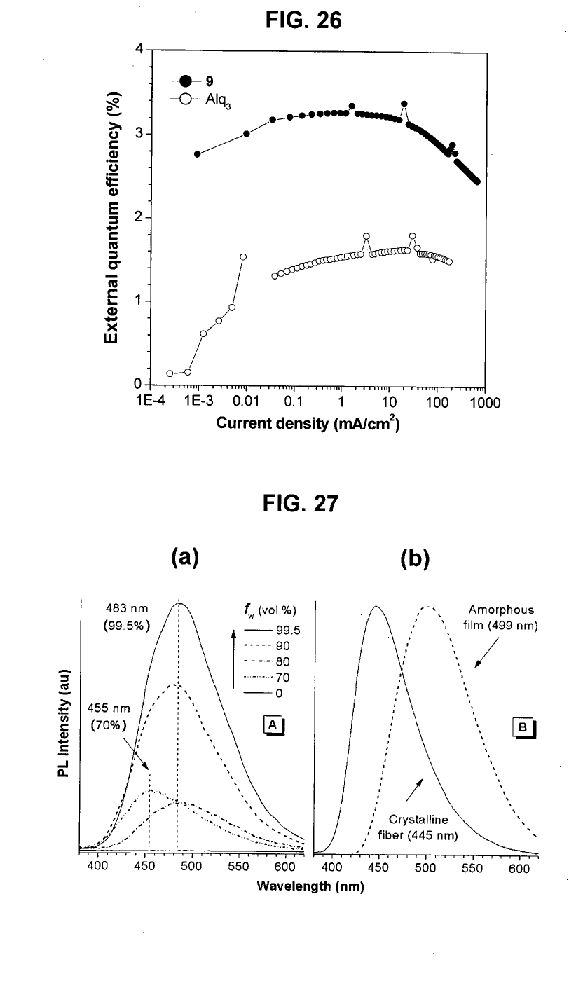

All the devices emit green lights in the range from 516 to 524 nm, which are red-shifted from the PL of their amorphous films (FIGS. 23 (a) and 23 (b)). In device I, 8 and 9 show low voltages of 3.9 and 5.3 V, exhibiting maximum luminance of 14,340 and 45,550 cd/m² at 15 V, and maximum current efficiency of 8.0 and 9.1 cd/A, respectively (FIGS. 20 (a) and 20 (b), respectively). The maximum external quantum efficiency attained by the device I reaches 2.9%. The EL performance of device II is even better. The device starts to emit at a lower voltage of 3.2 V and radiates more brilliantly with luminance up to 49,830 cd/cm² at 15 V. The maximum current efficiency and external quantum efficiency of the device are 10.2 cd/A and 3.3% (FIG. 26), respectively, which are much higher than those of the control device based on Alq₃ (FIGS. 20 (c) and 20 (d)), a well-known green emitter and electron-transport material. Such good EL performance should be attributed to not only its efficient solid-state PL, but also enhanced carrier mobility due to π-π stacking interactions of the pyrene rings. Although the device configuration is yet to be optimized, the excellent EL results are close to those of commercial pyrene-based light-emitting materials, clearly demonstrating the high potential of 8 and 9 as solid light-emitters for the construction of efficient EL devices.

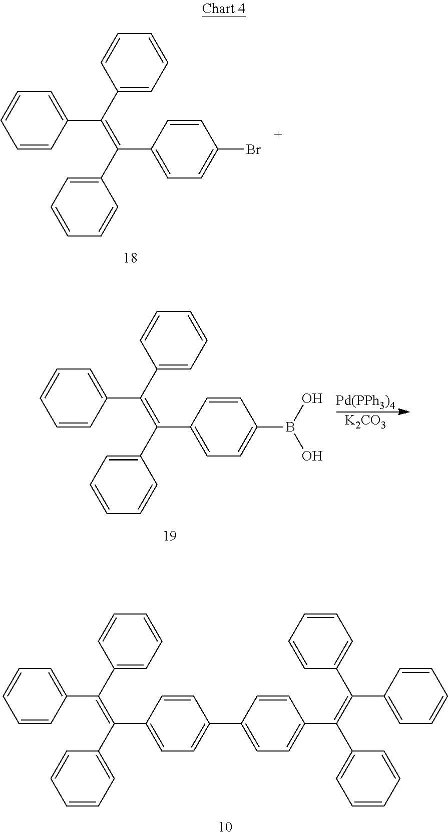

Chart 4 illustrates the synthesis of 10. Emission spectrum of the THF solution of 10 is a flat line parallel to the abscissa (FIG. 27 (a)), manifesting that 10 is non-fluorescent when it is molecularly dissolved as isolated species in its good solvent. A spectrum with a discernable peak cannot be obtained, which corroborates that the emission efficiency of 10 is intrinsically low and approaches nil (φ_{F,S}→0). However, in the THF/water mixtures with high fractions of water (F_{w}≧70%), 10 gives emission spectra with clear peaks. Since water is a non-solvent of 10, its molecules must have aggregated in the aqueous mixtures with high f_{w} ratios. The emission of 10 is thus induced by aggregation, confirming its anticipated AIE activity.

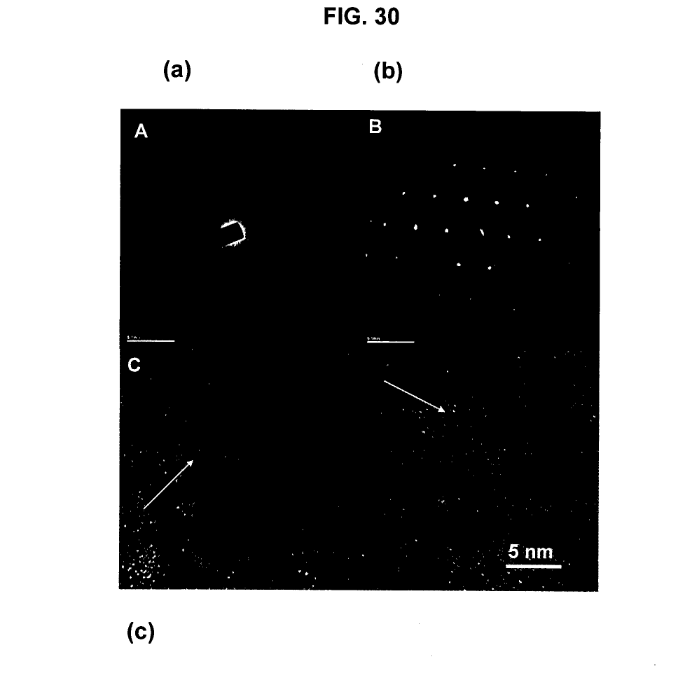

Closer check of the emission spectrum of 10 in the aqueous mixtures reveals that the emission maximum is bathochromically shifted from 450 nm to 484 nm when f_{w} becomes higher than 70%. This is probably due to a change in the morphology of the 10 aggregates. In a mixture with a lower f_{w} ratio (˜70%), the 10 molecules may slowly cluster together in an ordered fashion to form “bluer” crystalline aggregates. On the other hand, in a mixture with a higher f_{w} ratio (≧80%), the 10 molecules may abruptly heap up in a random way to form “redder” amorphous aggregates. This hypothesis is proved by the electron diffraction (ED) patterns of the aggregates: whilst clear diffraction spots are seen in the ED pattern of the aggregates formed in a mixture with f_{w}=70%, the aggregates formed in a mixture with f_{w}=80% give only a diffuse halo (FIG. 30).

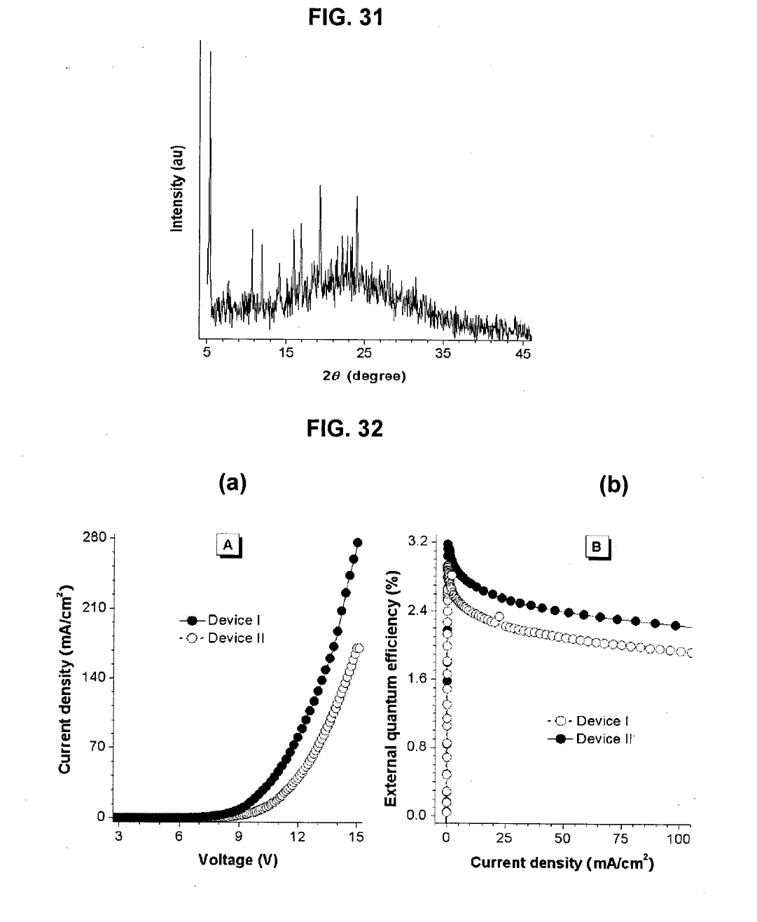

To validate that the crystalline aggregates emit bluer light than the amorphous ones, crystalline fibres of 10 was prepared by slow evaporation of its THF/ethanol solution and an amorphous film of 10 by spin-coating its THF solution onto a quartz plate. The crystalline nature of the fibres is verified by the sharp Bragg reflection peaks in their X-ray diffraction patterns (FIG. 31). Upon excitation, the crystalline fibres and amorphous film emit blue and green lights of 445 nm and 499 nm (FIG. 27 (b)) in quantum yields of 100% and 92% (measured with an integrating sphere), respectively. Thus, the luminogen crystallization does not only blue-shift emission colour but also increases emission efficiency. The φ_{F} value of unity indicates that the IMR process is completely inhibited when the 10 molecules are packed in the crystalline lattices.

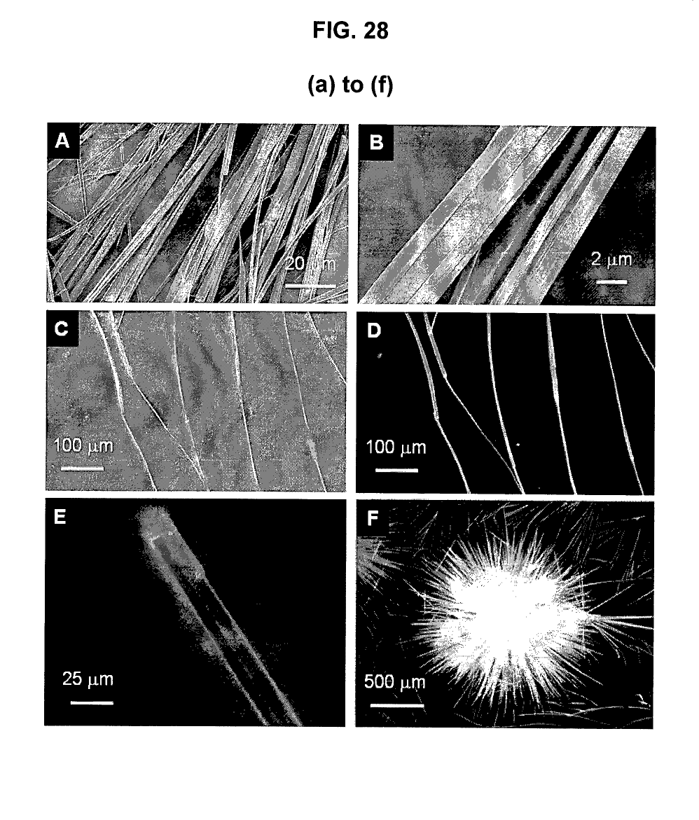

10 is capable of self-assembling. Its molecules pack in one-dimensional fashion to give crystalline microfibres when a solution of 10 containing a poor solvent (e.g., ethanol) in a Petri dish is slowly evaporated. Panels A and B of FIG. 28 show SEM images of the microfibres, which are several hundred microns in length and several microns in diameter. Most of the microfibres are smooth in surface, which is suggestive of a uniform arrangement of the luminogenic molecules. The fibres can also grow on a quartz plate when the plate is immersed into the dye solution. After solvent evaporation, fibres as long as several millimetres are readily formed, which can be observed even with naked eyes. The fibres can further assemble into thicker rods, as exemplified by the optical image shown in FIG. 28 (c). Panels (d)-(f) of FIG. 28 show fluorescence images of the wires of 10 with different sizes. The microwires are highly luminescent, emitting intense blue light upon photoexcitation. The φ_{F} value of the microwires is much higher than those of the organic nanowires reported by other groups (Chem. Eur. J. 2008, 14, 9577, J. Am. Chem. Soc. 2007, 129, 6978.), which may find high-tech applications in the fabrication of miniature electronic and photonic devices.

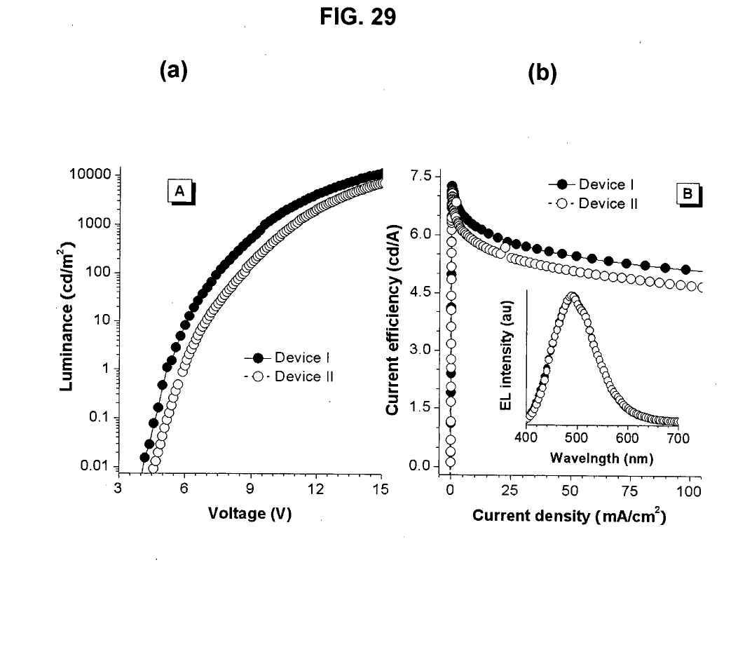

The highly efficient photoluminescence of 10 aggregates in the solid state prompted us to study its electroluminescence. Multilayer light-emitting diodes with configurations of ITO/NPB (60 nm)/10 (x)/TPBi (10 nm)/Alq₃ (y)/LiF (1 nm)/Al (100 nm) are fabricated, where x=20 nm, y=30 nm for device I and x=40 nm, y=10 nm for device II. In these EL devices, 10 works as a light emitter, NPB functions as a hole-transport material, and TPBi and Alq₃ serve as electron-transport materials. Both the EL devices emit a sky blue light of 488 nm (FIG. 29), a colour between those of the lights emitted by the amorphous film and crystalline fibres of 10, suggesting that the 10 layers in the EL devices contain both amorphous and crystalline aggregates. The devices do not only show identical emission spectra but also similar EL performances. The devices are turned on at low biases (down to ˜4 V) and radiate brilliantly with luminance up to 11180 cd/cm² at 15 V (FIG. 29 (a)). Current efficiency and external quantum efficiency of device I reach 7.26 cd/A and 3.17%, respectively, at a bias of 6 V (FIG. 29 (b), FIG. 32). Although the device configuration is yet to be optimized, the excellent EL data clearly demonstrate the great potential of 10 as a solid light-emitter in the construction of efficient EL devices.

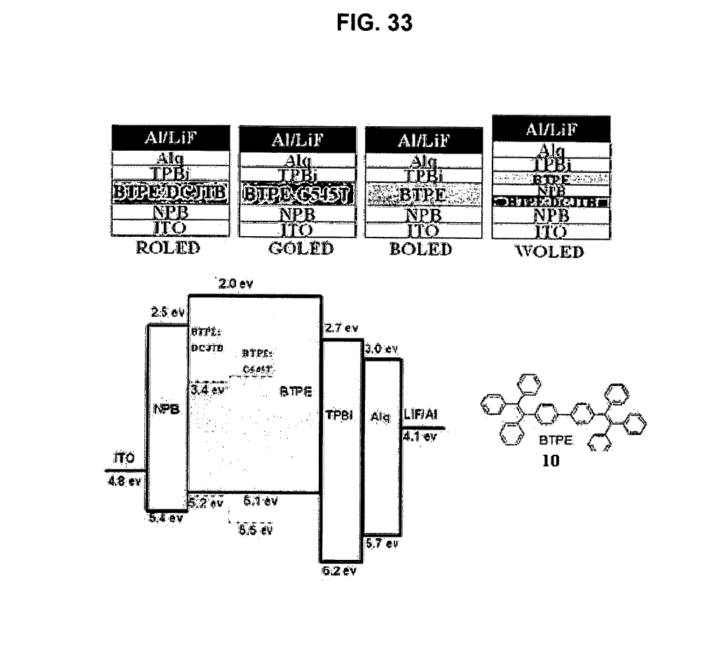

To investigate the EL property of 10, four kinds of devices were prepared on 80 nm thick ITO coated glass. The structures of the fabricated devices as well as the energy level and molecular structure of BTPE (10) are shown in FIG. 33. These devices contain a 20 nm thick 10 doped with 1% wt. DCJTB, a 20 nm thick 10 doped with 1% wt. C545T, a 20 nm thick BTPE, and a 20 nm thick BTPE combined with 1 nm thick BTPE doped with 1% wt. DCJTB were employed as the light-emitting layer for the R, G, B and WOLEDs, respectively. For the WOLEDs, a 2 nm thick NPB layer was inserted between the BTPE and BTPE:DCJTB serving as the electron-blocking layer. A 60 nm thick NPB, a 10 nm thick TPBi, and a 30 nm thick Alq₃, were used as hole-transporting, hole-blocking, and electron-transporting layers, respectively. All organic layers in the devices were thermally evaporated in sequence in a multi-source vacuum chamber at a base pressure of around 5×10⁻⁷ Torr. The samples were then transferred to the metal chamber without breaking vacuum for cathode deposition which is composed of 1 nm thick LiF capped with 100 nm thick Al.

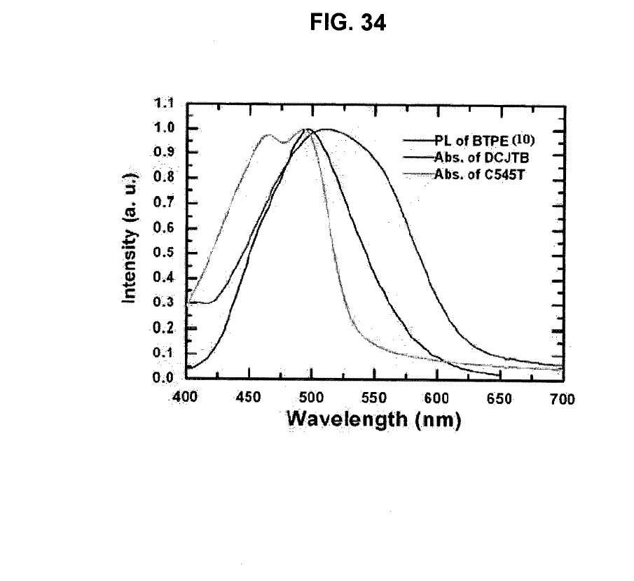

FIG. 34 shows the photoluminescent (PL) spectrum of amorphous thin film BTPE as well as the absorption spectrum of DCJTB and C5451. The PL emission of BTPE peaks at 492 nm, exhibiting a greenish-blue color. The fluorescent quantum yield (φ_{F}) of amorphous thin film BTPE is 92%, which implies that efficient BOLEDs may be obtained by using BTPE as an emitter. A bluer emission at 445 nm and higher φ_{F} of 100% can be obtained by crystallizing BTPE; in other words, instead of quenching like conventional fluorescent dyes, crystallization blue-shifts the emission spectrum and enhances the emission of BTPE, which is one of the properties of the novel AIE materials. The band-gap of BTPE is 3.1 eV as measured by cyclic voltammetry; such wide band-gap and high φ_{F} may render BTPE as a good host for fluorescent green and red dyes. As shown in FIG. 34, the PL spectrum of BTPE overlaps very well with the absorption spectrum of DCJTB and 0545T, indicating that effective Förster energy-transfer from BTPE to DCJTB or C545T may happen.

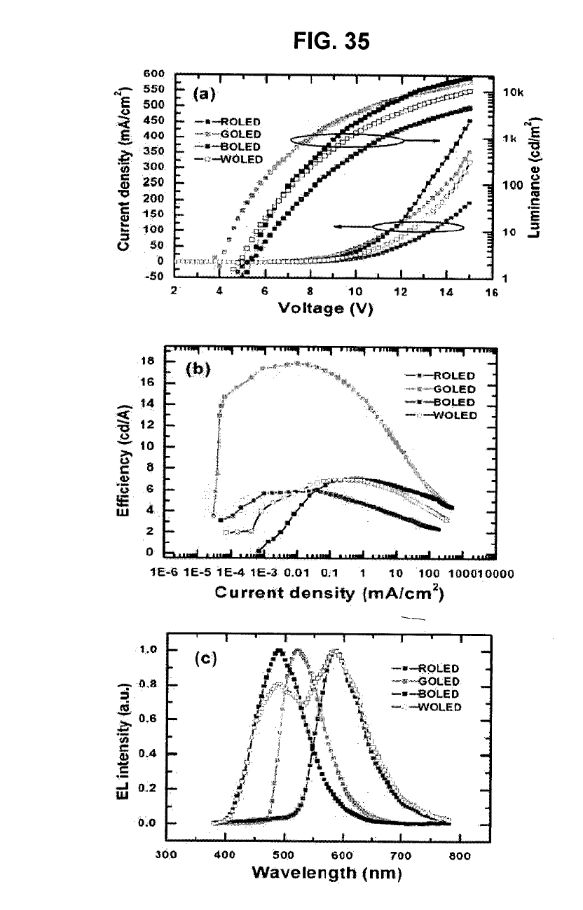

FIG. 35 shows the typical current density-luminance-voltage, current efficiency-current density characteristics and EL spectra of the devices. The non-doped BOLEDs employing BTPE as emitter directly show a turn on voltage at 1 cd/m² of 5 V. The luminance increases quickly with increased voltage, reaching 20,036 cd/m² at 15 V. The maximum current efficiency is 7.1 cd/A. By doping BTPE with red dye DCJTB and green dye C545T, the resulting ROLEDs and GOLEDs exhibit a substantially smaller current density and lower turn on voltage compared to the BOLEDs; for example, at a driving voltage of 15 V, the current density is 195 mA/cm² and 356 mA/cm² for the ROLEDs and GOLEDs respectively, significantly lower than 456 mA/cm² for the BOLEDs. Such reduced current density and turn on voltage of the ROLEDs and GOLEDs implies that besides effective energy transfers from BTPE, the excitons may form by directly trapping electrons and holes due to their narrower band-gap compared with BTPE (FIG. 33). This effective dual channel energy capturing of the dyes results in a maximum current efficiency of 5 cd/A and 18 cd/A for the ROLEDs and GOLEDs, respectively. The EL spectra shown in FIG. 35c further confirm this assumption. The non-doped BOLEDs exhibit a greenish-blue EL color with its peak at 488 nm; however, by doping BTPE with 1°/0 wt. C545T or DCJTB, the blue emission completely vanishes and is replaced by a 520 nm green or 588 nm red emission clearly demonstrating that the energy is completely transferred from BTPE to C545T or DCJTB.

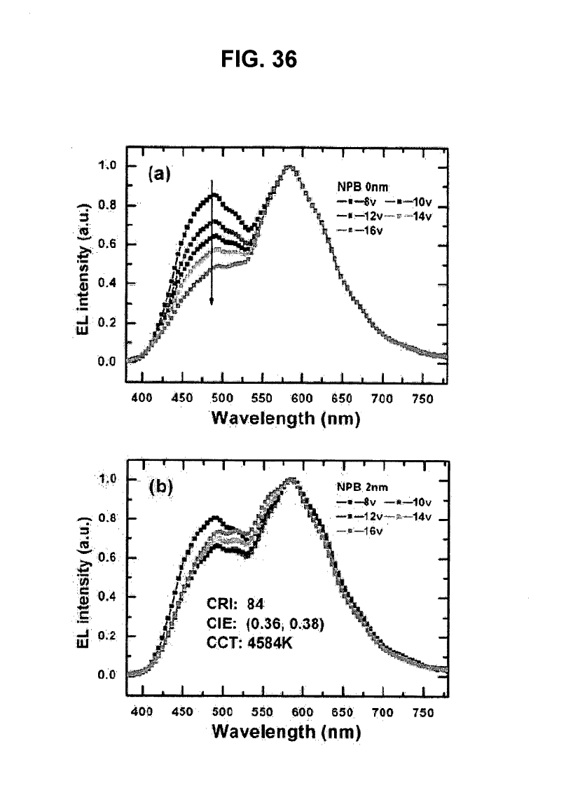

The simplified WOLEDs exhibit a turn on voltage of 4.5 V, a luminance of 10319 cd/m² at 15 V, and a maximum current efficiency of 7 cd/A. Two emission peaks at 488 nm and 588 nm, originating from BTPE and BTPE:DCJTB, can be clearly observed. FIG. 36 shows the EL spectra of the WOLEDs at different driving voltages. Without the NPB electron-blocking layer, the blue emission decreases as voltage is increased, mainly due to more excitons recombining in the BTPE:DCJTB layer with increased voltage, resulting in 1931 Commision International de L′Eclairage (CIE) coordinates and color correlate temperature (CCT) changing from (0.35, 0.37), 4832K at 8 V to (0.40, 0.41), 3688K at 16 V. With the help of NPB electron-blocking layer, the WOLEDs exhibit moderate color stability with CIE coordinates changing from (0.36, 0.38) to (0.38, 0.40) over a wide range of driving voltages. Moreover, a high color rendering index (CRI) of 84 is achieved by employing this simplified white light-emission layer containing only two kinds of materials.

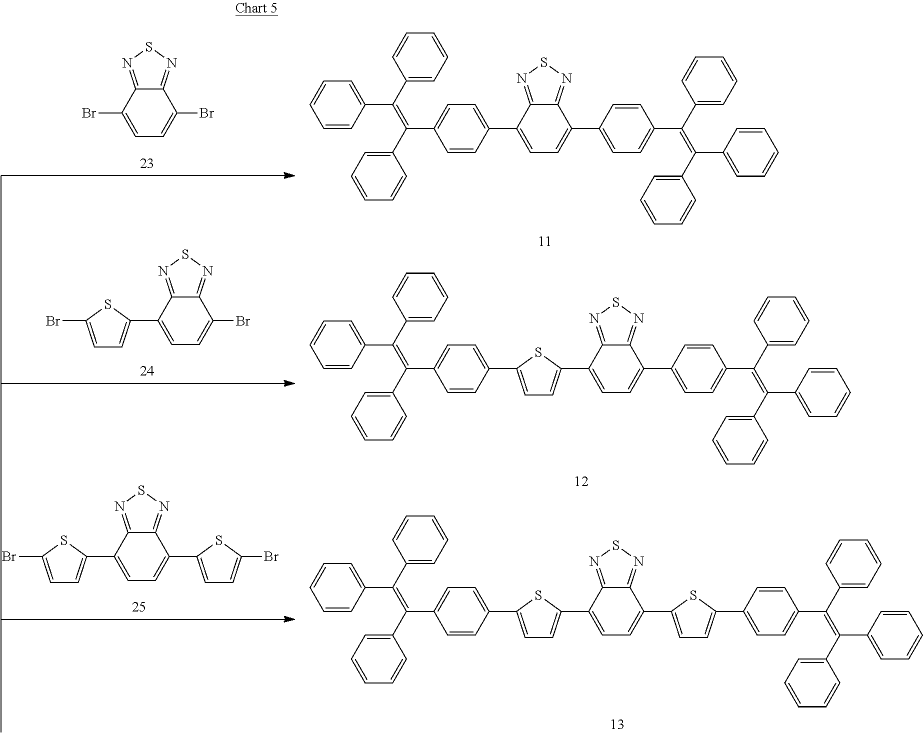

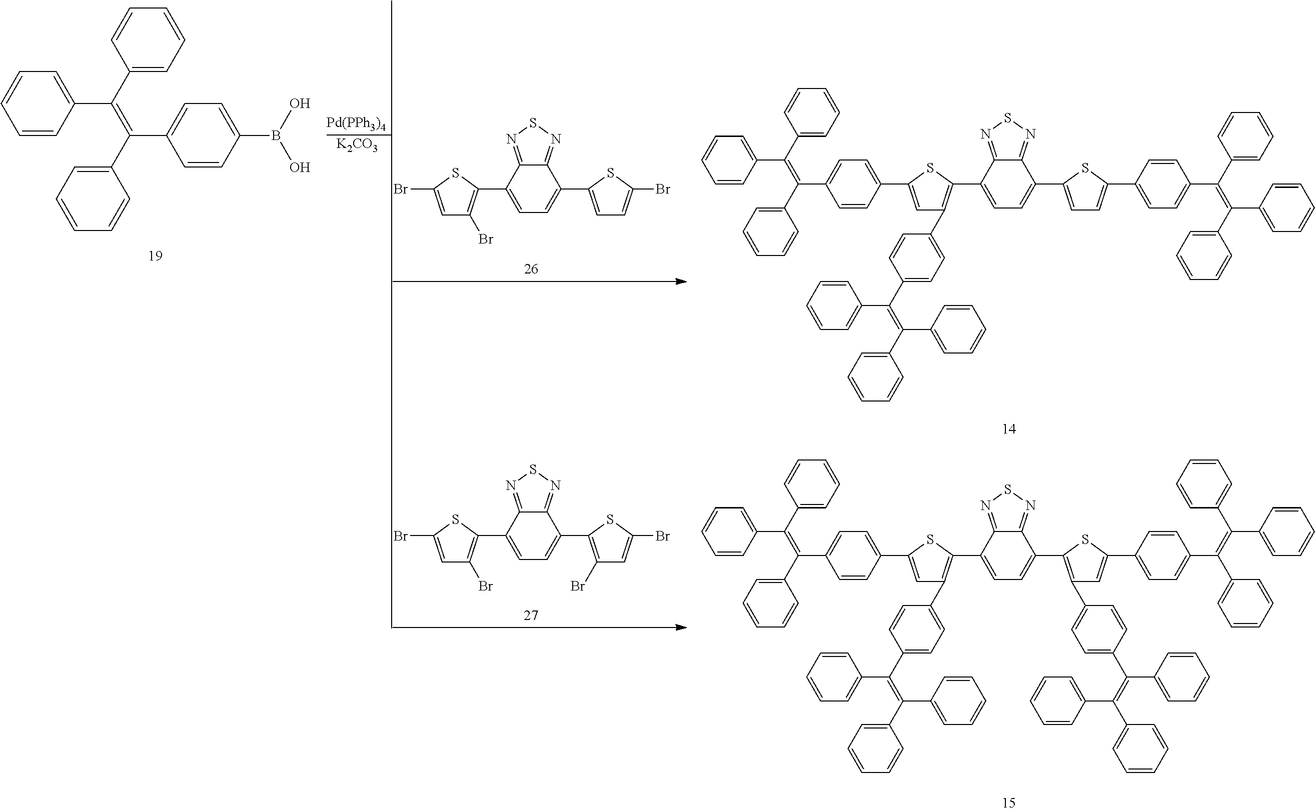

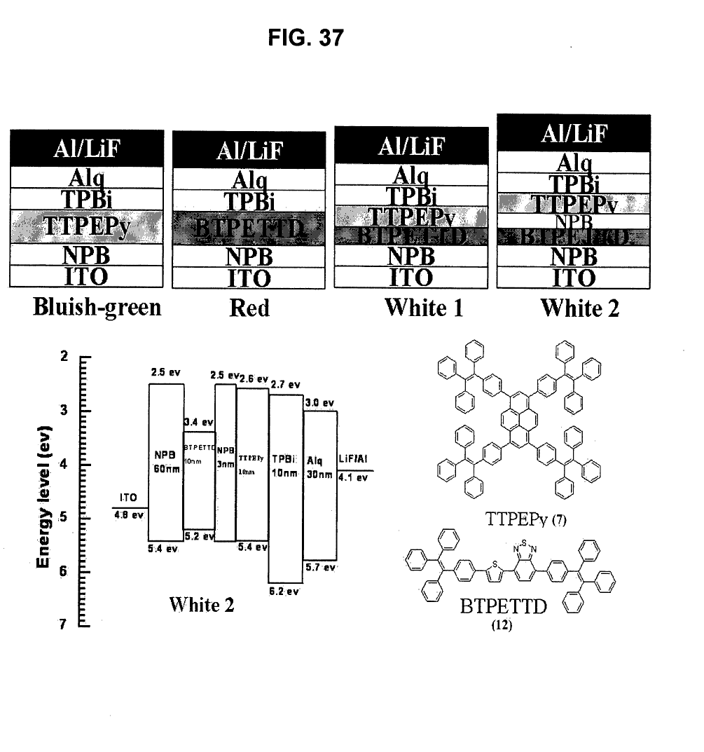

The EL properties of red emitter 12 and blue emitter 7 are investigated, and the structures of the fabricated devices as well as the energy level and molecular structures of the emitters are shown in FIG. 37. In these devices, a 20 nm thick TTPEPy (7), a 20 nm thick BTPETTD (12) and a 10 nm thick TTPEPy combined with 10 nm thick BTPETTD were employed as the light-emitting layer for the bluish-green, red and white OLEDs, respectively. For the white 2 OLEDs, a 3 nm thick NPB layer was inserted between the TTPEPy (7) and BTPETTD (12) serving as the electron-blocking layer. A 60 nm thick NPB, a 10 nm thick 2,2′,2″-(1,3,5-benzinetriyl)tris(1-phenyl-1-H-benzimidazole) (TPBi), and a 30 nm thick tris(8-hydroxyquinoline)aluminum (Alq₃), were used as hole-transporting, hole-blocking, and electron-transporting layers, respectively. All organic layers in the devices were thermally evaporated in sequence in a multi-source vacuum chamber at a base pressure of around 5×10⁻⁷ Torr. The samples were then transferred to the metal chamber without breaking vacuum for cathode deposition which composed of 1 nm thick LiF capped with 100 nm thick Al.

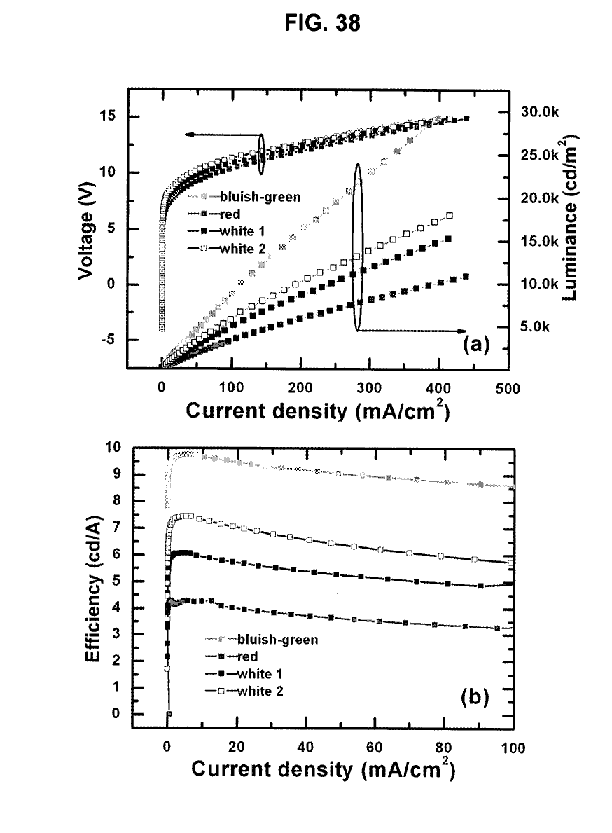

FIG. 38 (a) compares the typical voltage-luminance-current density characteristics of the devices. It is obvious that the bluish-green devices exhibit a substantially smaller current density compared to the red devices, mainly due to the larger energy band gap (FIG. 37) of TTPEPy compared to BTPETTD, resulting in larger carrier injection barriers in the bluish-green devices compared to that in the red devices. The current density of the white devices lies between that of the bluish-green and the red devices; white 2 devices with 3 nm thick NPB electron-blocking layer exhibit smaller current density compared to white 1 devices, which is expected since the introduction of the NPB layer blocks some of the electrons transporting from TTPEPy to BTPETTD. The luminance increases rapidly with increased current density for all devices. At a current density of 100 mA/cm², the bluish-green devices show a luminance of 8660 cd/m², significantly higher than 5700 cd/m², 5103 cd/m² and 3600 cd/m² for the white 2, white 1 and red devices, respectively.

As shown in FIG. 38 (b), the peak current efficiencies of the bluish-green and red devices are around 9.8 cd/A and 4.2 cd/A, respectively. The efficiencies of the white devices lie between that of the bluish-green and red devices. By introducing a 3 nm thick NPB electron-blocking layer, white 2 devices exhibit a peak current density of 7.4 cd/A, substantially higher than 6 cd/A for the white 1 devices. Such efficiency improvement is due to more even exciton distribution in white 2 devices. Without the NPB electron-blocking layer, most excitons recombine in the BTPETTD layer due to its lower energy band gap compared with TTPEPy (FIG. 37), resulting in lower efficiency due to the lower light-emitting efficiency of BTPETTD. With a 3 nm thick electron-blocking layer, more electrons are confined in the TTPEPy layer due to the poor electron-transporting property of NPB, leading to an even excitons distribution and hence higher efficiency in white 2 devices. In contrast to most of the doped-type fluorescent OLEDs, which suffer from tremendous efficiency roll-off at high doping concentration due to the notorious ACQ effect, all of the devices studied here show an impressive stability of efficiency due to their AIE nature. For instance, even at a high brightness of 5,000 cd/m², the efficiencies only slightly roll off to 9 cd/A, 6 cd/A, 5 cd/A and 3 cd/A for the blusih-green, white 2, white 1 and red devices, respectively.

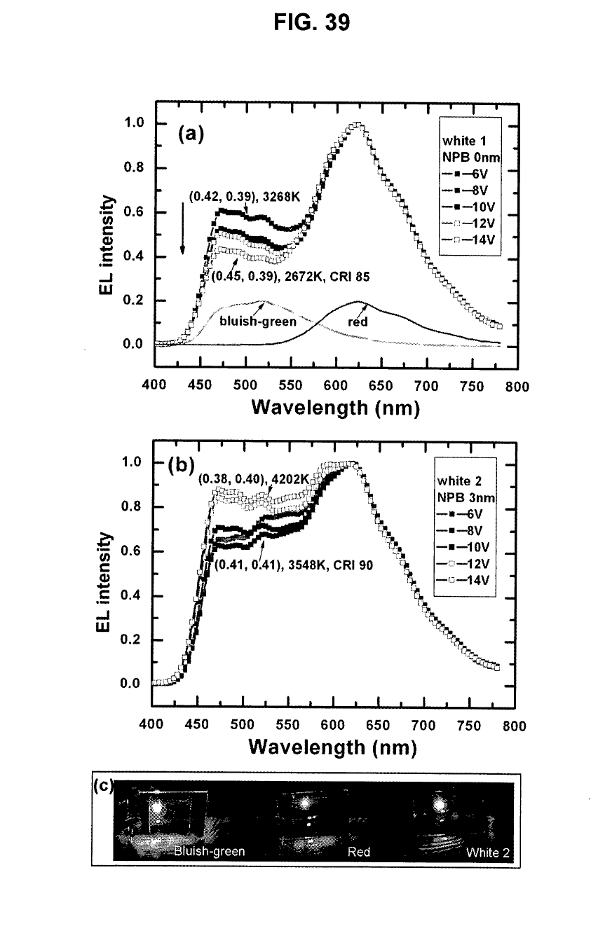

FIG. 39 (a) shows the spectra of the white 1 devices under different driving voltages as well as the spectra of the bluish-green devices and the red devices. Multiple-emission peaks center at 524 nm, 492 nm and 472 nm were observed for the bluish-green devices. The peak of 492 nm originates from TTPEPy, while other peaks are attributed to impurities. It should be noted that TTPEPy is only purified by boiling in THF followed by filtration, through which it is impossible to eliminate all of the metal catalysts. Provided cleaner TTPEPy, the efficiency would further improve. Indeed, a current efficiency of 12 cd/A and external quantum efficiency of 5% in bluish-green OLEDs have been achieved using cleaner TTPEPy. In spite of this disadvantage, high efficiency WOLEDs were obtained (FIG. 39 (c)). As shown in FIG. 39 (a), the bluish-green emission decreases as voltages increase, which is mainly due to more excitons recombining in the BTPETTD layer with increased voltage, resulting in 1931 Commision International de L′Eclairage (CIE) coordinates and color correlate temperature changing from (0.42, 0.39), 3268K at 6 V to (0.45, 0.39), 2672K at 14 V.

By introducing a 3 nm thick NPB electron-blocking layer, the blue-green emission is boosted significantly (FIG. 39 (b)), which clearly demonstrates that the NPB can block the electrons effectively. Interestingly, the bluish-green emission decreases as the voltages increase from 6 V to 8 V, and then gradually increases when the voltages change from 10 V to 14 V. It is known that the current is dominated by bulk space-charge-limited current at high voltages in organic semiconductors. For NPB, the electron current is very easy to approach the bulk limitation due to the extremely small electron trap densities. When the driving voltages are smaller than 8 V, the injected number of electrons is small and thus it is not sufficient to fill all of the electron traps of NPB; as a result, some of the injected electrons can pass NPB and recombine in the BTPETTD layer, leading to the reduced bluish-green emission with voltages increased. As driving voltages increase, more amounts of electrons are injected, which fill all of the electron traps of NPB, resulting in more electrons being confined in the TTPEPy layer, thus leading to a gradual increase of bluish-green emission. With the help of the NPB electron-blocking layer, the CIE coordinates and color correlate temperature shift close to the equivalent energy point, changing from (0.41, 0.41), 3548K at 8 V to (0.38, 0.40), 4202K at 14 V. Moreover, a high color rendering index (CRI) of 90 was achieved, owing to the broad and flat spectrum covering the entire visible spectrum. The key characteristics of the devices are listed in Table 10.

TABLE 10
Performance of the 7 and 12 based devices

| | Lₘₐₓ | η_{Lmax} | η_{Pmax} | CIE (x, y) | CIE (x, y) | CRI |
|---|---|---|---|---|---|---|
| Device | (cd/m2) | (cd/A) | (lm/W) | @6 V | @14 V | @14 V |
| Bluish- | 30000 | 9.8 | 6.0 | (0.26, | (0.26, 0.44) | — |
| green | | | | 0.44) | | |
| Red | 11000 | 4.2 | 2.7 | (0.61, | (0.61, 0.39) | — |
| | | | | 0.39) | | |
| White 1 | 15000 | 6.0 | 3.2 | (0.42, | (0.45, 0.39) | 85 |
| | | | | 0.39) | | |
| White 2 | 18000 | 7.4 | 4.0 | (0.40, | (0.38, 0.40) | 90 |
| | | | | 0.42) | | |

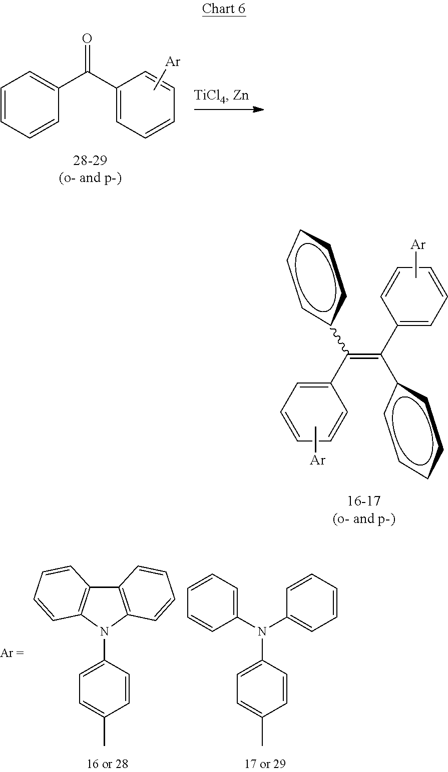

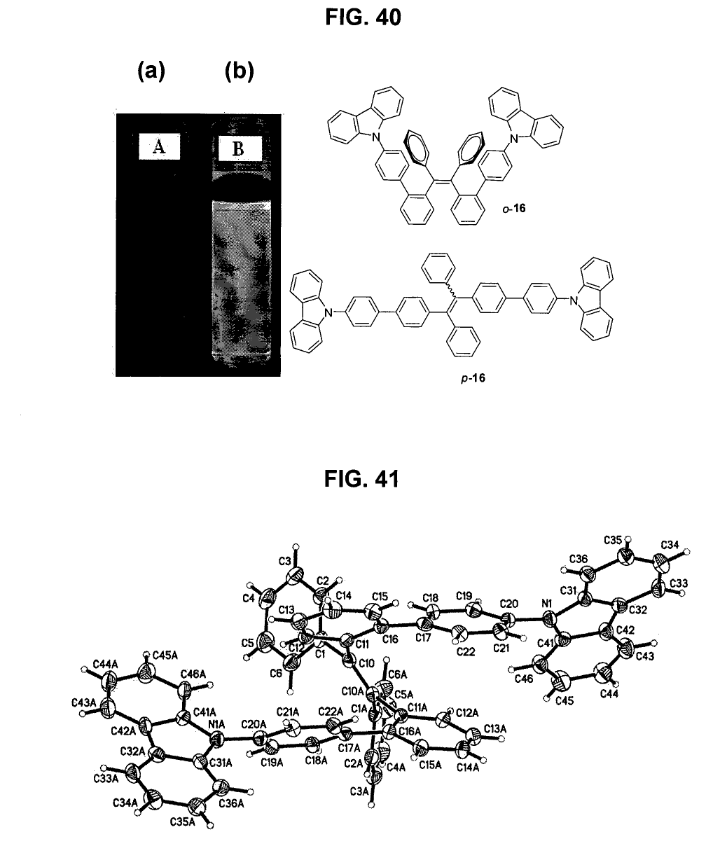

FIG. 40 compares the THF solution of o-16 and p-16 under UV irradiation. Due to the presence of steric group in o-16, the IMR of the molecule is minimized, resulting in fluorescence of its THF solution. As a comparison, p-16 has a similar structure but with the substitute at para-position and it is non-emissive in its THF solution.

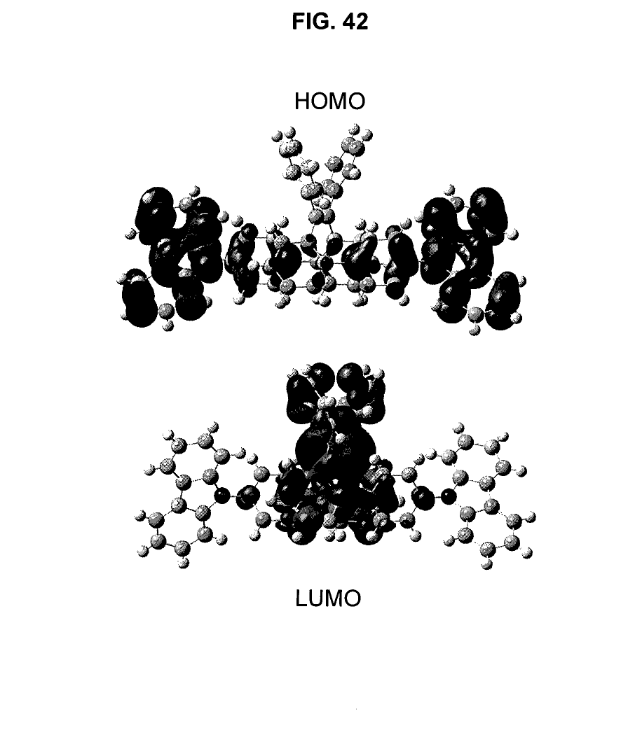

The crystal structure of o-16 is shown in FIG. 41. The calculated molecular orbitals of o-16 are displayed in FIG. 42.

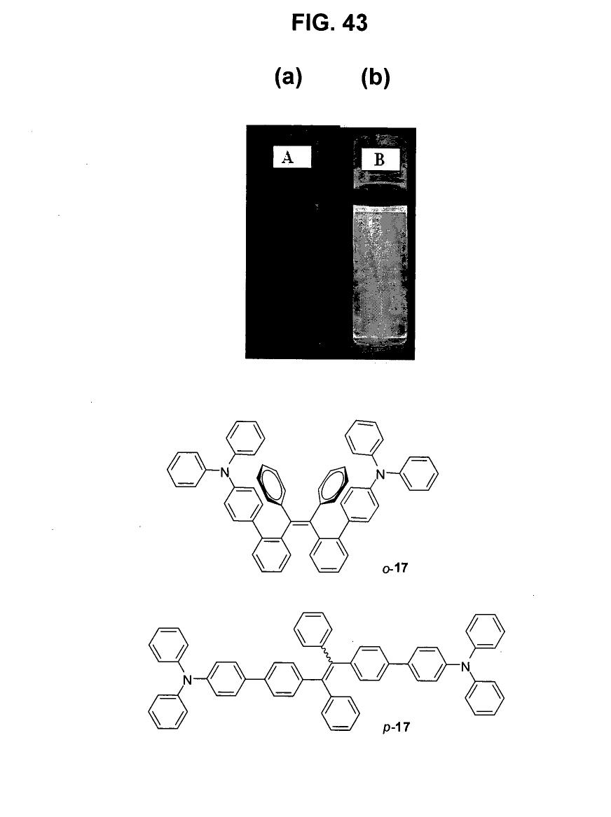

Compound 17 shows a similar phenomenon to 16. The THF solution of o-17 is emissive and p-17 is non-emissive. FIG. 43 shows the comparison between the two solutions, which proves that the IMR is a very important key to the photoluminescence behavior of the molecules.

### EXAMPLES

The present subject matter can be illustrated in further detail by the following examples. However, it should be noted that the scope of the present subject matter is not limited to the examples. They should be considered as merely being illustrative and representative for the present subject matter.

### Example 1

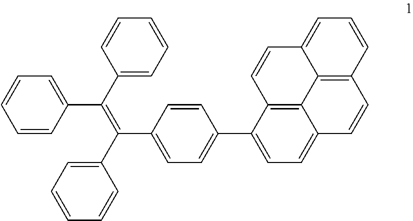

A mixture of 19 (1.0 mmol), 1-bromopyrene (1.1 mmol), Pd(PPh₃)₄ (0.05 mmol) and potassium carbonate (4.0 mmol) in 100 mL of toluene/ethanol/water (8/1/1 v/v/v) was heated to reflux for 24 h under nitrogen. After filtration and solvent evaporation, the residue was purified by silica-gel column chromatography using a hexane/dichloromethane or ethyl acetate mixture as eluent.

Characterization Data: White solid; yield 63%. m.p.: 303° C. ¹H NMR (300 MHz, CD₂Cl₂), δ(TMS, ppm): 8.21-8.16 (m, 3H), 8.11-7.93 (m, 6H), 7.37 (d, 2H, J=8.7 Hz), 7.22-7.08 (m, 17). ¹³C NMR (75 MHz, CD₂Cl₂), δ(TMS, ppm): 144.5, 144.4, 144.3, 143.4, 142.1, 141.4, 139.8, 138.3, 132.2, 131.7, 131.2, 130.6, 129.1, 128.4, 128.2, 128.1, 128.0, 127.2, 126.7, 126.0, 125.7, 125.6, 125.4, 125.3. MS (MALDI-TOF): m/z 532.2513 (M⁺, calcd 532.2191). Anal. Calcd for C₄₂H₂₈: C, 94.70; H, 5.30. Found: C, 94.64; H, 5.29.

### Example 2

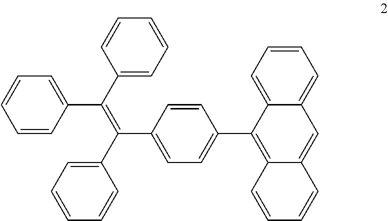

A mixture of 19 (1.0 mmol), 9-bromoanthracene (1.1 mmol), Pd(PPh₃)₄ (0.05 mmol), and potassium carbonate (4.0 mmol) in 100 mL of toluene/ethanol/water (8/1/1 v/v/v) was heated to reflux for 24 h under nitrogen. After filtration and solvent evaporation, the residue was purified by silica-gel column chromatography using hexane/dichloromethane or ethyl acetate mixture as eluent.

Characterization Data: White solid; yield 69%. m.p.: 301° C. ¹H NMR (300 to MHz, CD₂Cl₂), δ(TMS, ppm): 8.45 (s, 1H), 8.03 (d, 2H, J=8.4 Hz), 7.59 (d, 2H, J=8.7 Hz), 7.48-7.43 (m, 2H), 7.38-7.33 (m, 2H), 7.25-7.13 (M, 19H). ¹³C NMR (75 MHz, CD₂Cl₂), δ(TMS, ppm): 144.6, 144.4, 144.2, 143.9, 142.3, 137.6, 137.5, 132.2, 132.1, 132.03, 132.00, 131.9, 131.2, 130.8, 129.0, 128.51, 128.45, 128.4, 127.4, 127.3, 127.1, 126.0, 125.9. MS (MALDI-TOF): m/z 508.2436 (M⁺, calcd 508.2191). Anal. Calcd for C₄₀H₂₈: C, 94.45; H, 5.55. Found: C, 94.14; H, 5.57.

### Example 3

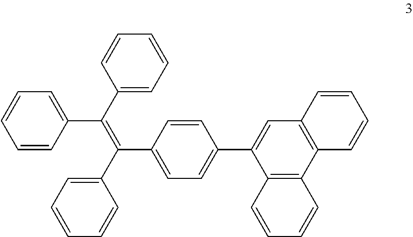

A mixture of 19 (1.0 mmol), 9-bromophenanthrene (1.1 mmol), Pd(PPh₃)₄ (0.05 mmol), and potassium carbonate (4.0 mmol) in 100 mL of toluene/ethanol/water (8/1/1 v/v/v) was heated to reflux for 24 h under nitrogen. After filtration and solvent evaporation, the residue was purified by silica-gel column chromatography using hexane/dichloromethane or ethyl acetate mixture as eluent.

Characterization Data: White solid; yield 80%. m.p.: 200° C. ¹H NMR (300 MHz, CD₂Cl₂), δ(TMS, ppm): 8.76 (d, 1H, J=7.8 Hz), 8.71 (d, 1H, J=8.4 Hz), 7.90-7.83 (m, 2H), 7.69-7.51 (m, 5H), 7.29 (d, 2H, J=7.8 Hz), 7.20-7.08 (m, 17H). ¹³C NMR (75 MHz, CD₂Cl₂), δ(TMS, ppm): 144.5, 144.4, 143.7, 142.1, 141.5, 139.5, 139.2, 132.3, 132.1, 132.0, 131.9, 131.7, 131.3, 130.6, 130.1, 129.3, 128.5, 128.4, 128.0, 127.6, 127.5, 127.3, 127.2, 123.6, 123.2. MS (MALDI-TOF): m/z 508.2397 (M⁺, calcd 508.2191). Anal. Calcd for C₄₀H₂₈: C, 94.45; H, 5.55. Found: C, 94.06; H, 5.57.

### Example 4

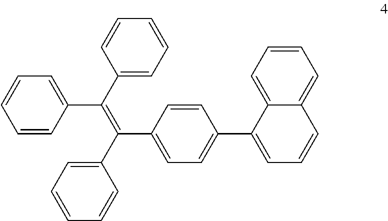

A mixture of 19 (1.0 mmol), 1-bromonaphthalene (1.1 mmol), Pd(PPh₃)₄ (0.05 mmol), and potassium carbonate (4.0 mmol) in 100 mL of toluene/ethanol/water (8/1/1 v/v/v) was heated to reflux for 24 h under nitrogen. After filtration and solvent evaporation, the residue was purified by silica-gel column chromatography using hexane/dichloromethane or ethyl acetate mixture as eluent.

Characterization data: White solid; yield 85%. m.p.: 190° C. ¹H NMR (300 MHz, CD₂Cl₂), δ(TMS, ppm): 7.89-7.79 (m, 3H), 7.51-7.36 (m, 4H), 7.24-7.08 (m, 19H). ¹³C NMR (75 MHz, CD₂Cl₂), δ(TMS, ppm): 144.4, 144.3, 143.3, 141.9, 141.3, 140.5, 139.3, 134.4, 132.1, 131.9, 131.8, 131.6, 129.9, 128.8, 128.3, 128.1, 127.3, 127.0, 126.5, 126.4, 126.3, 125.9. MS (MALDI-TOF): m/z 458.2551 (M⁺, calcd 458.2035). Anal. Calcd for C₃₆H₂₆: C, 94.29; H, 5.71. Found: C, 94.09; H, 5.82.

### Example 5

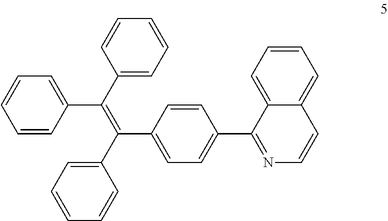

A mixture of 19 (1.0 mmol), 1-bromoisoquinoline (1.1 mmol), Pd(PPh₃)₄ (0.05 mmol), and potassium carbonate (4.0 mmol) in 100 mL of toluene/ethanol/water (8/1/1 v/v/v) was heated to reflux for 24 h under nitrogen. After filtration and solvent evaporation, the residue was purified by silica-gel column chromatography using hexane/dichloromethane or ethyl acetate mixture as eluent.

Characterization data: Fawn solid; yield 82%. m.p.: 195° C. ¹H NMR (300 MHz, to CD₂Cl₂), δ(TMS, ppm): 8.53 (d, 1H, J=5.7 Hz), 8.01 (d, 1H, J=9.6 Hz), 7.87 (d, 1H, J=7.8 Hz), 7.70-7.65 (m, 1H), 7.61 (d, 1H, J=5.7 Hz), 7.55-7.49 (m, 1H), 7.43 (d, 2H, J=9.0 Hz), 7.20-7.06 (m, 17H). ¹³C NMR (75 MHz, CD₂Cl₂), δ(TMS, ppm): 160.9, 144.9, 144.5, 144.4, 142.9, 142.3, 141.4, 138.4, 137.6, 132.1, 132.0, 131.9, 131.8, 130.7, 130.1, 128.5, 128.4, 128.1, 127.8, 127.7, 127.4, 127.3, 120.5. MS (MALDI-TOF): m/z 460.1752 (M⁺, calcd 459.1987). Anal. Calcd for C₃₅H₂₅N: C, 91.47; H, 5.48; N, 3.05. Found: C, 91.24; H, 5.56; N, 3.06.

### Example 6

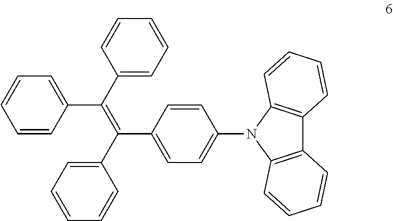

n-Butyllithium (1.6 M in hexane, 3.8 mL, 6 mmol) was added dropwise into a THF solution (50 mL) of 18 (2 g, 5 mmol) at −78° C. After stirring at −78° C. for 3 h, iodine (1.4 g, 5.5 mmol) was added into the solution in three portions. After warmed to room temperature and stirred for 2 h, the mixture was poured into water and extracted with dichloromethane. The organic layer was washed by saturated sodium thiosulfate solution and water, and dried over magnesium sulfate. After filtration and solvent evaporation, the crude product 20 was purified by flash silica-gel column chromatography using hexane as eluent. Compound 20 was then added into a solution of carbazole (1 g, 6 mmol), copper (0.32 g, 5 mmol), potassium carbonate (1 g, 7.5 mmol), and 18-crown-6 (0.027 g, 0.1 mmol) in 80 mL DMF, and stirred at 170° C. for 24 h under nitrogen. The reaction mixture was cooled to room temperature and filtered. The filtrate was poured into water, extracted with dichloromethane. The organic layer was washed by water and dried over magnesium sulfate. After filtration and solvent evaporation, the residue was purified by silica-gel column chromatography using hexane/dichloromethane as eluent.

Characterization data: White solid; yield 32%. m.p.: 205° C. ¹H NMR (300 MHz, CD₂Cl₂), δ(TMS, ppm): 8.13-8.07 (m, 4H), 7.45-7.41 (m, 6H), 7.40-7.10 (m, 17H). ¹³C NMR (75 MHz, CD₂Cl₂), δ(TMS, ppm): 144.2, 144.1, 144.0, 143.6, 142.5, 141.4, 140.8, 140.1, 136.4, 133.4, 132.1, 128.4, 127.4, 126.8, 126.5, 124.0, 121.0, 120.9, 120.5, 120.1, 111.3, 110.5. MS (MALDI-TOF): m/z 497.3266 (M⁺, calcd 497.2143). Anal. Calcd for C₃₈H₂₇N: C, 91.72; H, 5.47; N, 2.81. Found: C, 91.55; H, 5.60; N, 2.64.

### Example 7

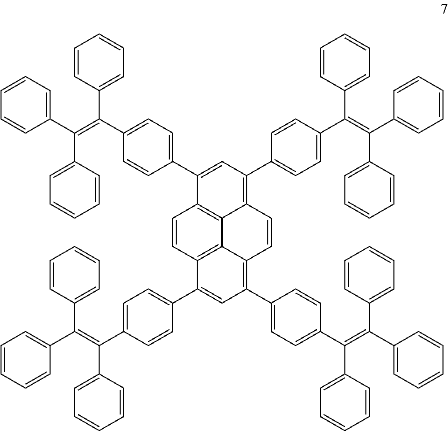

A mixture of 19 (2.3 g, 6 mmol), 1,3,6,8-tetrabromopyrene (0.52 g, 1 mmol), Pd(PPh₃)₄ (200 mg, 0.2 mmol), and potassium carbonate (2.8 g, 20 mmol) in 120 mL of degassed toluene/ethanol/water (8:2:2 v/v/v) was heated to reflux for 24 h under nitrogen. The precipitate was filtrated and washed with water, acetone, and tetrahydrofuran. After dried under vacuum, the product was purified by sublimation under vacuum. A pale green solid was obtained in 50% yield (0.76 g). The product was partially dissolved in toluene and benzene. No NMR spectra were obtained due to its limited solubility in organic solvents.

Characterization data: MS (MALDI-TOF): m/z 1524.2351 [(M+H)⁺, calcd 1524.6450)]. Anal. Calcd for C₁₂₀H₈₂: C, 94.58; H, 5.42. Found: C, 94.29; H, 5.70.

### Example 8

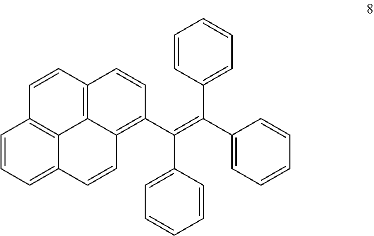

To a solution of diphenylmethane (1 g, 6 mmol) in dry THF (30 mL) was added dropwise a 1.6 M solution of n-butyllithium in hexane (3.7 mL, 6 mmol) at 0° C. under a nitrogen atmosphere. After stirred for 1 h at 0° C., the resultant orange-red solution was transferred slowly to a solution of pyrenophenone (1.5 g, 5 mmol) in THF (20 mL) at 0° C. The reaction mixture was allowed to warm to room temperature and stirred for 6 h. The reaction was quenched with the addition of an aqueous solution of ammonium chloride. The organic layer was extracted with dichloromethane and the combined organic layers were washed with a saturated brine solution and dried over anhydrous magnesium sulfate. After filtration and solvent evaporation, the resultant crude alcohol with excess diphenylmethane was dissolved in about 50 mL of toluene and a catalytic amount of p-toluenesulphonic acid (0.25 g, 1.3 mmol) was then added. After refluxed for 6 h, the mixture was cooled to room temperature and washed with saturated brine solution and water, and dried over anhydrous magnesium sulfate. After filtration and solvent evaporation, the residue was purified by silica-gel column chromatography using n-hexane/dichloromethane as eluent. Pale yellow solid of 8 was obtained in 72% yield (1.6 g).

Characterization data: ¹H NMR (300 MHz, CDCl₃), δ(TMS, ppm): 8.29 (d, 1H, J=9.3 Hz), 8.15-8.08 (m, 2H), 8.03-7.91 (m, 5H), 7.82 (d, 1H, J=7.8 Hz), 7.24-7.20 (m, 5H), 7.06-9.67 (m, 7H), 6.83-6.80 (m, 3H). ¹³C NMR (75 MHz, CDCl₃), δ(TMS, ppm): 144.3, 144.23, 144.19, 144.0, 140.1, 139.7, 139.3, 132.2, 131.9, 131.6, 131.4, 131.1, 130.5, 130.9, 128.6, 128.4, 128.1, 128.0, 127.7, 127.5, 127.1, 126.5, 126.2, 125.6, 125.5, 125.2. HRMS (MALDI-TOF): m/z 456.2043 (M⁺, calcd 456.1878). Anal. Calcd for C₃₆H₂₄: C, 94.70; H, 5.30. Found: C, 94.58; H, 5.51. m.p.: 203° C.

### Example 9

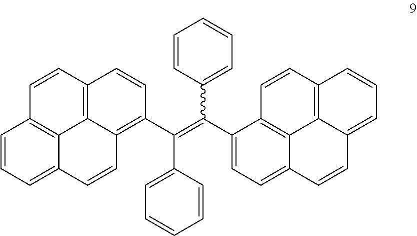

To a solution of pyrenophenone (1.5 g, 5 mmol), zinc dust (0.65 g, 10 mmol) in 50 mL dry THF was added dropwise of titanium(IV) chloride (0.95 g, 5 mmol) under nitrogen at −78° C. After stirring for 20 min, the reaction mixtures were warmed to room temperature and then heated to reflux for 12 h. The reaction mixture was then cooled to room temperature and poured into water. The organic layer was extracted with dichloromethane, and the combined organic layers were washed with saturated brine solution and water and dried over anhydrous magnesium sulfate. After filtration and solvent evaporation, the residue was purified by silica-gel column chromatography using n-hexane/dichloromethane as eluent.

Characterization data: Pale yellow solid of 9 was obtained in 56% yield (0.81 g). ¹H NMR (300 MHz, CDCl₃), δ(TMS, ppm): 8.48-8.40 (m, 2H), 8.20-7.95 (m, 16H), 7.01-6.96 (m, 4H), 6.83-6.74 (m, 6H). HRMS (MALDI-TOF): m/z 580.4069 (M⁺, calcd 580.2129). Anal. Calcd for C₄₆H₂₈: C, 95.14; H, 4.86. Found: C, 94.87; H, 4.96. m.p.: 279° C.

### Example 10

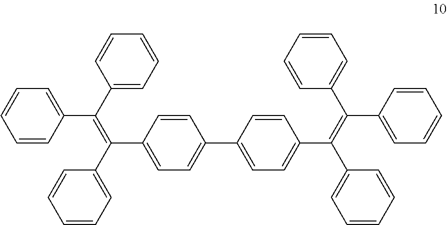

A mixture of 18 (1.0 mmol), 19 (1.1 mmol), Pd(PPh₃)₄ (0.05 mmol), and potassium carbonate (4.0 mmol) in 100 mL of toluene/ethanol/water (8/1/1 v/v/v) was heated to reflux for 24 h under nitrogen. After filtration and solvent evaporation, the residue was purified by silica-gel column chromatography using hexane as eluent.

Characterization data: M.p.: 290° C. ¹H NMR (300 MHz, CD₂Cl₂), δ (TMS, ppm): 7.31 (d, 4H, J=8.4 Hz), 7.00-7.11 (m, 34H). ¹³C NMR (75 MHz, CD2Cl₂), δ(TMS, ppm): 144.44, 144.41, 144.39, 143.40, 141.70, 141.19, 138.90, 132.42, 132.07, 132.02, 128.43, 128.34, 128.30, 127.14, 127.08, 126.57. MS (MALDI-TOF): m/z 662.2151 (M⁺, 662.2974).

### Example 11

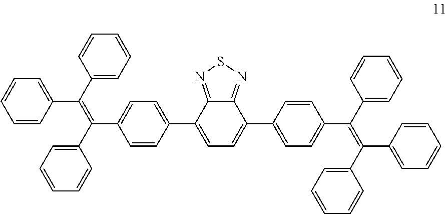

A mixture of 19 (2.2 mmol), 23 (1.0 mmol), Pd(PPh₃)₄ (0.1 mmol), and potassium carbonate (8.0 mmol) in 100 mL of toluene/ethanol/water (8/1/1 v/v/v) was heated to reflux for 24 h under nitrogen. After filtration and solvent evaporation, the residue was purified by silica-gel column chromatography using hexane/dichloromethane or ethyl acetate mixture as eluent.

Characterization data: HRMS (MALDI-TOF): m/z 796.3184 (M⁺, calcd 796.2912).

### Example 12

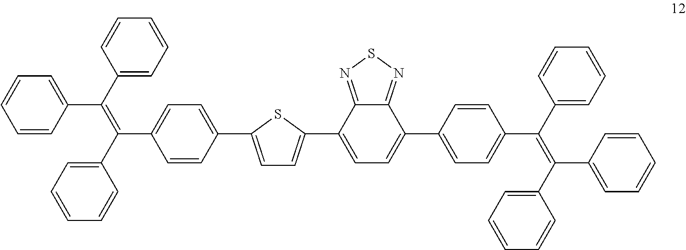

A mixture of 19 (2.2 mmol), 24 (1.0 mmol), Pd(PPh₃)₄ (0.1 mmol), and potassium carbonate (8.0 mmol) in 100 mL of toluene/ethanol/water (8/1/1 v/v/v) was heated to reflux for 24 h under nitrogen. After filtration and solvent evaporation, the residue was purified by silica-gel column chromatography using hexane/dichloromethane or ethyl acetate mixture as eluent.

Characterization data: HRMS (MALDI-TOF): m/z 878.2714 (M⁺, calcd 878.2789).

### Example 13

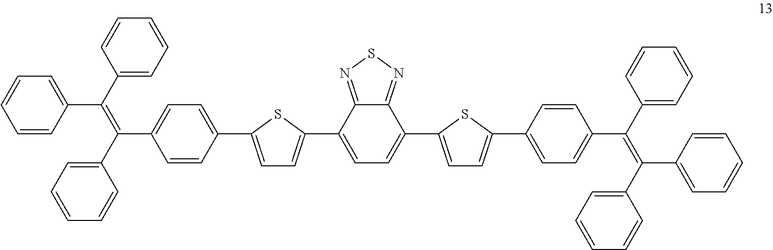

A mixture of 19 (2.2 mmol), 25 (1.0 mmol), Pd(PPh₃)₄ (0.1 mmol), and potassium carbonate (8.0 mmol) in 100 mL of toluene/ethanol/water (8/1/1 v/v/v) was heated to reflux for 24 h under nitrogen. After filtration and solvent evaporation, the residue was purified by silica-gel column chromatography using hexane/dichloromethane or ethyl acetate mixture as eluent.

Characterization data: HRMS (MALDI-TOF): m/z 960.2310 (M⁺, calcd 960.2667).

### Example 14

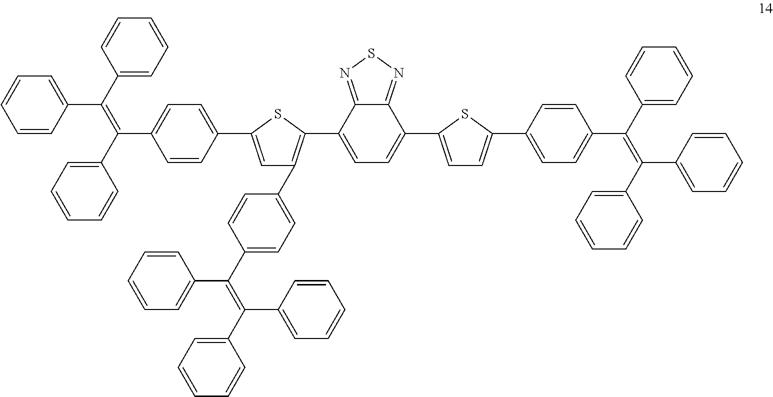

A mixture of 19 (2.2 mmol), 26 (1.0 mmol), Pd(PPh₃)₄ (0.1 mmol), and potassium carbonate (8.0 mmol) in 100 mL of toluene/ethanol/water (8/1/1 v/v/v) was heated to reflux for 24 h under nitrogen. After filtration and solvent evaporation, the residue was purified by silica-gel column chromatography using hexane/dichloromethane or ethyl acetate mixture as eluent.

Characterization data: HRMS (MALDI-TOF): m/z 1291.4797 (M⁺, calcd 1290.4075).

### Example 15

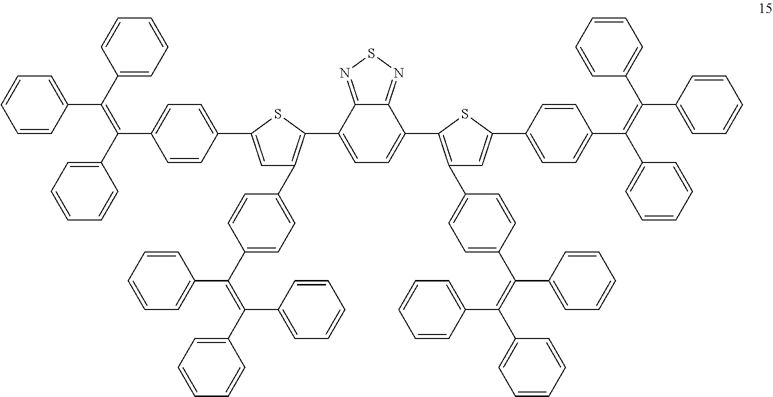

A mixture of 19 (2.2 mmol), 27 (1.0 mmol), Pd(PPh₃)₄ (0.1 mmol), and potassium carbonate (8.0 mmol) in 100 mL of toluene/ethanol/water (8/1/1 v/v/v) was heated to reflux for 24 h under nitrogen. After filtration and solvent evaporation, the residue was purified by silica-gel column chromatography using hexane/dichloromethane or ethyl acetate mixture as eluent.

Characterization data: HRMS (MALDI-TOF): m/z 1621.9682 (M⁺, calcd 1621.5517).

### Example 16

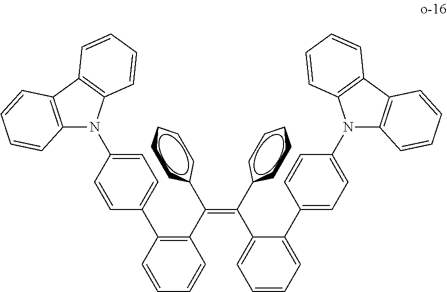

To a solution of o-28 (5 mmol), zinc dust (0.65 g, 10 mmol) in 50 mL dry THF was added dropwise of titanium(IV) chloride (0.95 g, 5 mmol) under nitrogen at −78° C. After stirring for 20 min, the reaction mixtures were warmed to room temperature and then heated to reflux for 12 h. The reaction mixture was then cooled to room temperature and poured into water. The organic layer was extracted with dichloromethane, and the combined organic layers were washed with saturated brine solution and water and dried over anhydrous magnesium sulfate. After filtration and solvent evaporation, the residue was purified by silica-gel column chromatography using n-hexane/dichloromethane as eluent.

Characterization data: HRMS (MALDI-TOF): m/z 814.1420 (M⁺, calcd 814.3348).

### Example 17

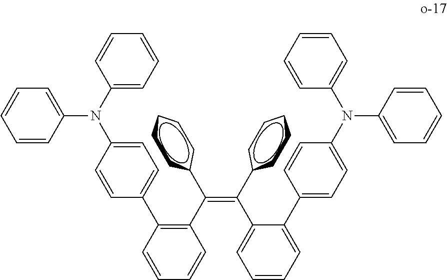

To a solution of o-29 (5 mmol), zinc dust (0.65 g, 10 mmol) in 50 mL dry THF was added dropwise of titanium(IV) chloride (0.95 g, 5 mmol) under nitrogen at −78° C. After stirring for 20 min, the reaction mixtures were warmed to room temperature and then heated to reflux for 12 h. The reaction mixture was then cooled to room temperature and poured into water. The organic layer was extracted with dichloromethane, and the combined organic layers were washed with saturated brine solution and water and dried over anhydrous magnesium sulfate. After filtration and solvent evaporation, the residue was purified by silica-gel column chromatography using n-hexane/dichloromethane as eluent.

Characterization data: HRMS (MALDI-TOF): m/z 818.3617 (M⁺, calcd 818.3661).

### Example 18

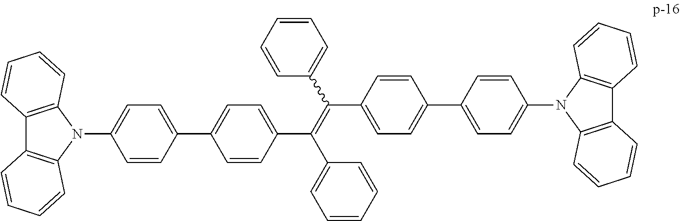

To a solution of p-28 (5 mmol), zinc dust (0.65 g, 10 mmol) in 50 mL dry THF was added dropwise of titanium(IV) chloride (0.95 g, 5 mmol) under nitrogen at −78° C. After stirring for 20 min, the reaction mixtures were warmed to room temperature and then heated to reflux for 12 h. The reaction mixture was then cooled to room temperature and poured into water. The organic layer was extracted with dichloromethane, and the combined organic layers were washed with saturated brine solution and water and dried over anhydrous magnesium sulfate. After filtration and solvent evaporation, the residue was purified by silica-gel column chromatography using n-hexane/dichloromethane as eluent.

Characterization data: HRMS (MALDI-TOF): m/z 814.8936 (M⁺, calcd 814.3348).

### Example 19

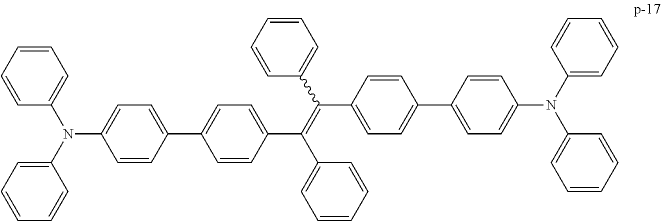

To a solution of p-29 (5 mmol), zinc dust (0.65 g, 10 mmol) in 50 mL dry THF was added dropwise of titanium(IV) chloride (0.95 g, 5 mmol) under nitrogen at −78° C. After stirring for 20 min, the reaction mixtures were warmed to room temperature and then heated to reflux for 12 h. The reaction mixture was then cooled to room temperature and poured into water. The organic layer was extracted with dichloromethane, and the combined organic layers were washed with saturated brine solution and water and dried over anhydrous magnesium sulfate. After filtration and solvent evaporation, the residue was purified by silica-gel column chromatography using n-hexane/dichloromethane as eluent.

Characterization data: HRMS (MALDI-TOF): m/z 819.4875 (M⁺, calcd 818.3661).

While the foregoing written description of the present subject matter enables one of ordinary skill to make and use what is considered presently to be the best mode thereof, the person of ordinary skill will understand and appreciate the existence of variations, combinations, and equivalents of the specific embodiment, method, and examples herein. The present subject matter should therefore not be limited by the above described embodiments, methods, and examples, but by all embodiments and methods within the scope and spirit of the invention.

What is claimed is:

1. A light emitting material comprising one or more moieties of formula (1a): 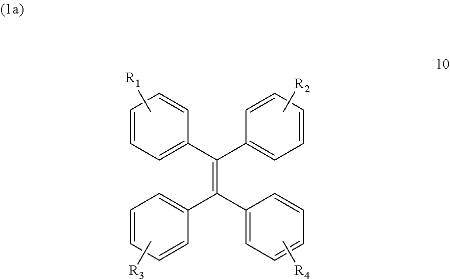
wherein R₁, R₂, R₃, and R₄, each independently of one another at each occurrence, are hydrogen or any organic or organometallic groups, with the proviso that at least one of R₁ to R₄ is not hydrogen; and when R₁ and R₄, or R₂ and R₃, are hydrogen, the other two R₂ and R₃, or R₁ and R₄, are not phenyl groups.

2. The light emitting material of claim 1 having a molecular weight of at least about 300.

3. The light emitting material of claim 1 having a molecular weight of between about 300 and about 3000.

4. The light emitting material of claim 1, wherein R₁, R₂, R₃, and R₄, each independently of one another at each occurrence, are hydrogen, optionally substituted C₂-C₆ alkyl, optionally substituted vinyl, optionally substituted acetyl, optionally substituted aryl, optionally substituted heteroaryl, optionally substituted cycloalkyl, optionally substituted heterocycloalkyl, or optionally substituted heteroalkyl.

5. The light emitting material of claim 1, wherein R₁, R₂, R₃, and R₄, each independently of one another at each occurrence, are selected from the group consisting of: 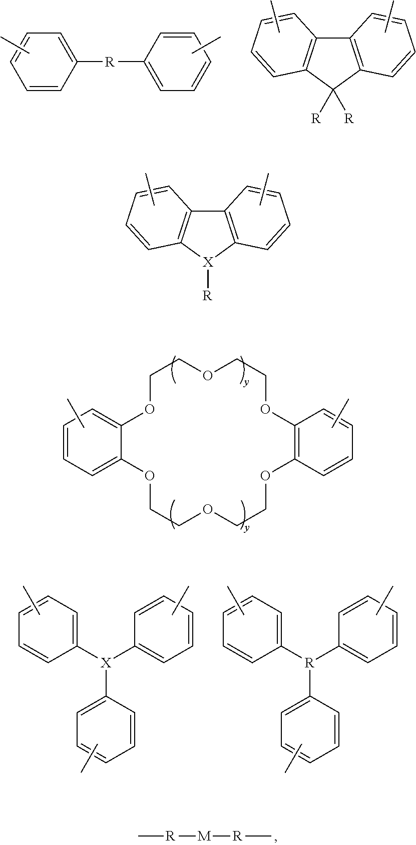
and hydrogen, wherein X is a heteroatom; y is an integer and is ≧1; R is alkyl, vinyl, acetyl, aryl, heteroaryl, cycloalkyl, heterocycloalkyl, or heteroalkyl that is optionally substituted;
and M is a metal or organometallic compound.

6. The light emitting material of claim 1 selected from the group consisting of:

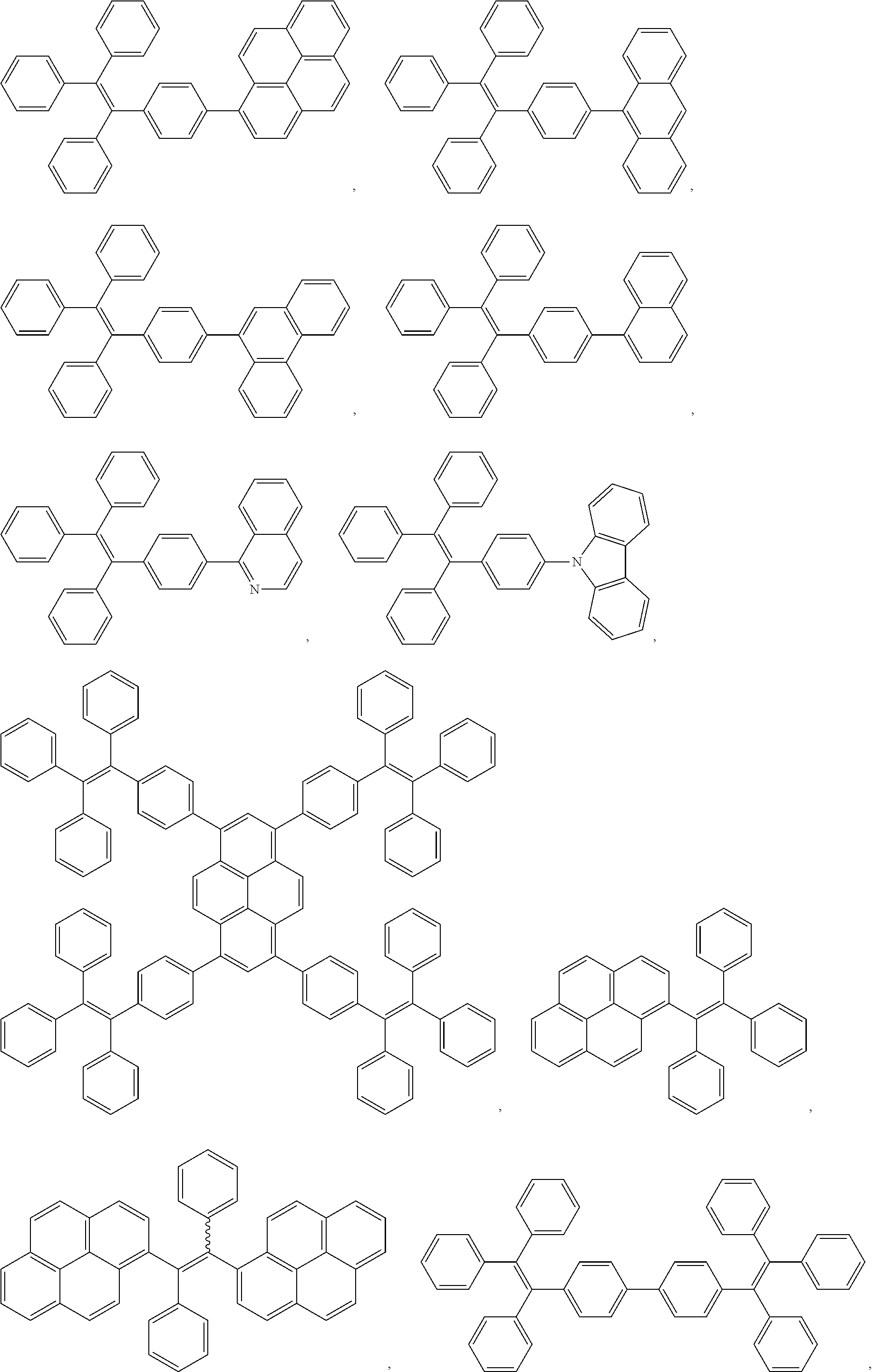

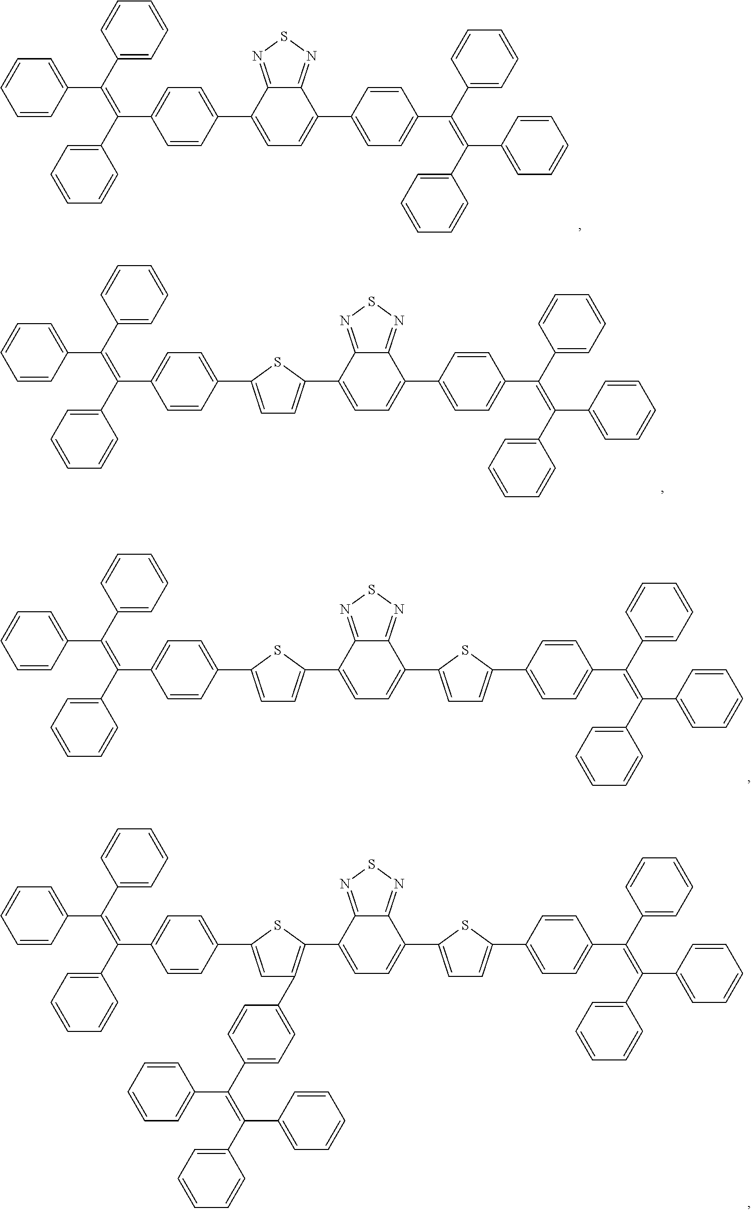

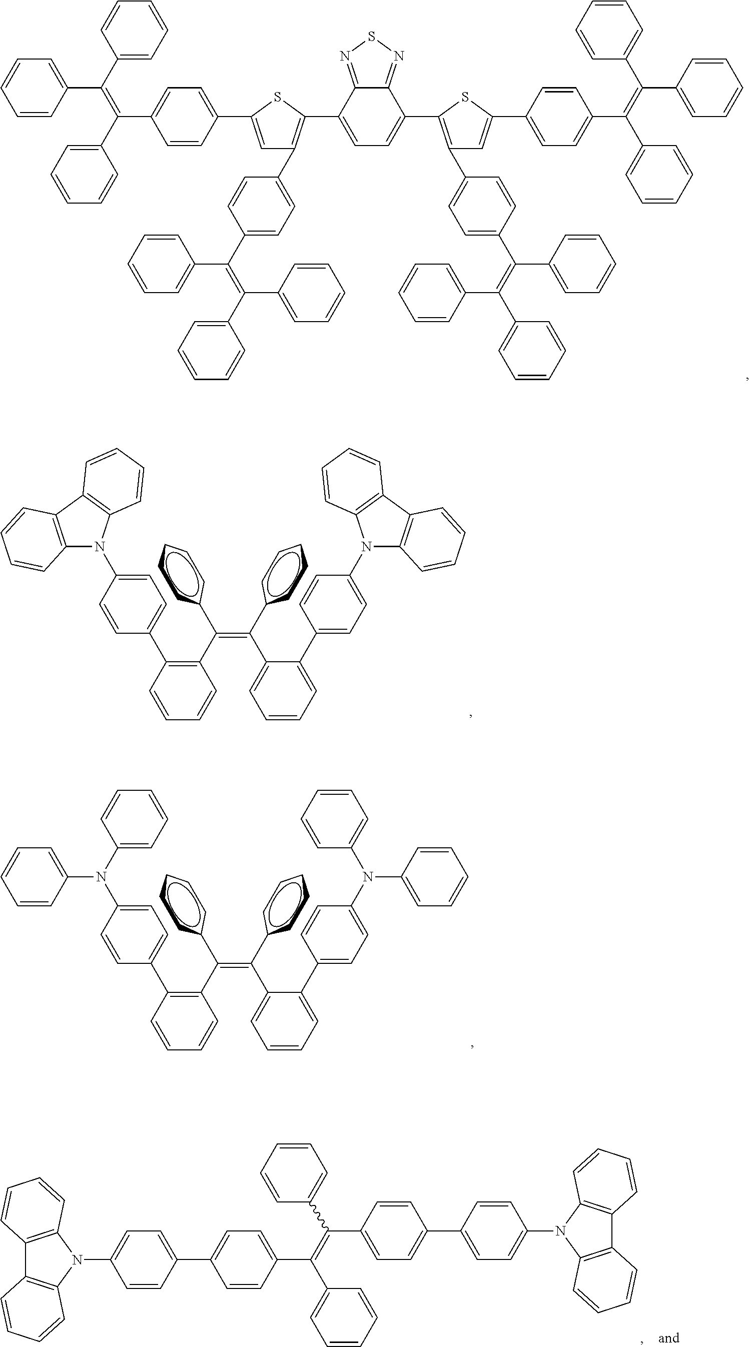

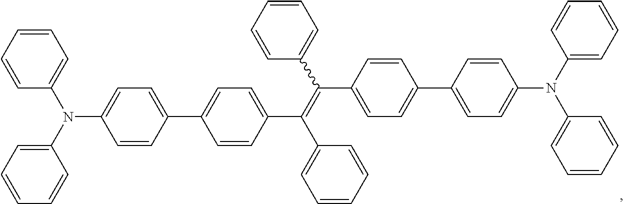

7. The light emitting material of claim 1, in solid or crystalline form.

8. Use of the light emitting material of claim 1 for the preparation of an emitting layer of an organic light emitting device (OLED).

9. An electroluminescent (EL) device comprising the material of claim 1.

10. A light emitting device comprising the material of claim 1.

11. The electroluminescent device of claim 9, using electricity as an energy source.

12. The light emitting device of claim 10, using electricity as an energy source electricity.

13. An organic light emitting device (OLED) comprising an anode, a cathode and an organic layer located therebetween, said organic layer comprising the material of claim 1.

14. The light emitting device of claim 28, wherein the material is a solid light-emitter.

15. A method of preparing a light emitting device comprising an anode, a cathode and one or more organic layers located between the anode and the cathode, which comprises thermally evaporating the organic layer in sequence in a multi-source vacuum chamber at a base pressure, wherein the organic layer comprises the material of claim 1.
